(19) United States
(12) Patent Application Publication
(10) Pub. No.: US 2024/0189387 A1
(43) Pub. Date: Jun. 13, 2024

(54) DOSING REGIMENS FOR HEMATOPOIETIC STEM CELL MOBILIZATION FOR STEM CELL TRANSPLANTS IN MULTIPLE MYELOMA PATIENTS

(71) Applicant: Ensoma, Inc., Boston, MA (US)

(72) Inventor: Veit Schmelmer, Natick, MA (US)

(21) Appl. No.: 18/468,506

(22) Filed: Sep. 15, 2023

Related U.S. Application Data
(63) Continuation of application No. PCT/US2022/020510, filed on Mar. 16, 2022.
(60) Provisional application No. 63/161,882, filed on Mar. 16, 2021.
(60) Provisional application No. 63/196,610, filed on Jun. 3, 2021.
(60) Provisional application No. 63/273,856, filed on Oct. 29, 2021.

(51) Int. Cl. A61K 38/17; A61K 9/00; A61K 31/395; A61P 35/00

(52) U.S. Cl. CPC A61K 38/17; A61K 9/0019; A61K 31/395; A61P 35/00

(57) ABSTRACT

The invention provides compositions and methods useful for mobilizing populations of hematopoietic stem and progenitor cells within a donor, such as a donor with multiple myeloma, as well as for determining whether samples of mobilized cells are suitable for release for ex vivo expansion and/or therapeutic use. In accordance with the compositions and methods described herein, mobilized hematopoietic stem and progenitor cells can be withdrawn from a patient with multiple myeloma for performing an autologous stem cell transplant to treat the patient's multiple myeloma.

### RELATED APPLICATIONS

This application is a continuation of International Application No. PCT/US2022/020510 filed Mar. 16, 2022, which claims the benefit of and priority to U.S. Provisional Patent Application No. 63/161,882, filed on Mar. 16, 2021, U.S. Provisional Patent Application No. 63/196,610, filed on Jun. 3, 2021, and U.S. Provisional Patent Application No. 63/273,856, filed on Oct. 29, 2021, the entire contents of each of which are incorporated herein by reference.

### SEQUENCE LISTING

The instant application contains a Sequence Listing which has been submitted electronically in XML file format and is hereby incorporated by reference in its entirety. Said XML copy, created on Nov. 20, 2023, is named MGA-010WOC1_SL.xml and is 5,535 bytes in size.

### FIELD OF THE INVENTION

The invention relates to the mobilization of hematopoietic stem and progenitor cells from a donor, such as a human donor with a myeloma/hematologic malignancy. The mobilized hematopoietic stem and progenitor cells are useful for performing a transplant, such as an autologous transplant.

### BACKGROUND OF THE INVENTION

Despite advances in the medicinal arts, there remains a demand for treating pathologies of the hematopoietic system, such as diseases of a particular blood cell, metabolic disorders, cancers, and autoimmune conditions, among others. While hematopoietic stem cells have significant therapeutic potential, a limitation that has hindered their use in the clinic has been the difficulty associated with releasing hematopoietic stem cells from the bone marrow into the peripheral blood of a donor, from which the hematopoietic stem cells may be isolated for infusion into a patient.

A further limitation is that up to 80% of mobilized peripheral blood (mPB) allogeneic recipients will experience graft-versus-host disease (GVHD). Despite the higher levels of CD3+ T cells in mPB grafts compared to bone marrow transplants, the level of acute GVHD observed following transplant of HLA-matched mPB is comparable to HLA-matched bone marrow. One explanation is that G-CSF mobilized grafts contain myeloid-derived suppressor cells (MDSCs) possessing potent immunosuppressive properties capable of inhibiting T cell proliferation in vitro. The percentage of MDSCs is variable in grafts mobilized with G-CSF, and clinical data suggest that patients transplanted with mPB grafts that contain higher numbers of MDSCs may have better outcomes including lower rates of acute GVHD (Vendramin et al., (2014) BBMT 20(12):2049-2055).

Accordingly, there is currently a need for compositions and methods for promoting the mobilization of hematopoietic stem and progenitor cells, and particularly for methods of identifying populations of mobilized cells that are suitable for therapeutic use. There is also a need for compositions and methods for promoting the mobilization of hematopoietic stem and progenitor cells that consistently produce higher numbers of MDSCs than do prior art methods.

### SUMMARY OF THE INVENTION

The present invention provides compositions and methods for mobilizing hematopoietic stem and progenitor cells in a subject. For example, the subject may be a hematopoietic stem and progenitor cell donor (i.e., a donor), such as a mammalian donor, and particularly a human donor. The invention additionally provides compositions and methods for the treatment of disorders, such as stem cell disorders or hematologic disorders, in a patient, such as a human patient. In various embodiments, the donor and the patient are the same individual and therefore, the methods and compositions disclosed herein are useful for performing an autologous stem cell transplant. In particular embodiments, the donor and the patient are the same individual and has been previously diagnosed with multiple myeloma.

Using the compositions and methods described herein, a C-X-C chemokine receptor type 2 (CXCR2) agonist, such as Gro-β or a variant thereof, such as a truncated form of Gro-β (e.g., Gro-β T), as described herein, optionally in combination with a C-X-C chemokine receptor type 4 (CXCR4) antagonist, such as 1,1′-[1,4-phenylenebis(methylene)]-bis-1,4,8,11-tetra-azacyclotetradecane or a variant thereof, may be administered to a subject in amounts sufficient to mobilize hematopoietic stem and progenitor cells. The compositions and methods described herein thus enable the mobilization of hematopoietic stem and progenitor cells in a donor, which may then be isolated from a donor for therapeutic use.

In some embodiments, the hematopoietic stem or progenitor cells may be mobilized from the bone marrow of the donor to the peripheral blood, from which the hematopoietic stem or progenitor cells may be collected and/or isolated. Upon collection of the mobilized cells, the withdrawn hematopoietic stem or progenitor cells may then be infused into a patient, which may be the donor or another subject, such as a subject that is HLA-matched to the donor, for the treatment of one or more pathologies of the hematopoietic system. In some embodiments, the withdrawn hematopoietic stem or progenitor cells are first expanded ex vivo prior to infusion of these cells, and/or progeny thereof, into the patient. Thus, the populations of mobilized hematopoietic stem and progenitor cells produced using the compositions and methods described herein are particularly suitable for hematopoietic stem cell transplantation therapy, optionally preceded by ex vivo expansion in order to increase the quantity of hematopoietic stem and progenitor cells available for infusion into a patient.

As described herein, hematopoietic stem cells are capable of differentiating into a multitude of cell types in the hematopoietic lineage and can thus be administered to a patient in order to populate or repopulate a cell type that is defective or deficient in the patient. The patient may be one, for example, that is suffering from one or more blood disorders, such as an autoimmune disease, cancer, hemoglobinopathy, or other hematopoietic pathology, and is therefore in need of hematopoietic stem cell transplantation. The invention thus provides methods of treating a variety of hematopoietic conditions, such as sickle cell anemia, thalassemia, Fanconi anemia, Wiskott-Aldrich syndrome, adenosine deaminase deficiency-severe combined immunodeficiency, metachromatic leukodystrophy, Diamond-Blackfan anemia and Schwachman-Diamond syndrome, human immunodeficiency virus infection, and acquired immune deficiency syndrome, as well as cancers and autoimmune diseases, among others.

Disclosed herein is a method of mobilizing a population of hematopoietic stem or progenitor cells from the bone marrow of a mammalian donor into peripheral blood, the method comprising administering to the donor a CXCR2 agonist selected from the group consisting of Gro-β, Gro-β T, and variants thereof at a dose of from about 0.001 mg/kg to about 0.1 mg/kg, wherein the donor was previously diagnosed with multiple myeloma. In various embodiments, the dose is from greater than about 0.015 mg/kg to less than about 0.05 mg/kg. In various embodiments, the CXCR2 agonist comprises Gro-β T. In various embodiments, the CXCR2 agonist is administered at a dose of about 0.03 mg/kg.

In various embodiments, the CXCR2 agonist is administered intravenously. In various embodiments, the CXCR2 agonist is administered over a period of time of less than about 20 minutes. In various embodiments, the CXCR2 agonist is administered over a period of time of less than about 10 minutes. In various embodiments, the CXCR2 agonist is administered over a period of time between about 3 to about 10 minutes. In various embodiments, administration of the CXCR2 agonist over a period of time of less than about 20 minutes results in fewer adverse effects in the donor in comparison to a donor who receives administration of the CXCR2 agonist over a period of time greater than about 20 minutes.

In various embodiments, the method described herein further comprises administering to the donor a CXCR4 antagonist. In various embodiments, the CXCR4 antagonist is plerixafor. In various embodiments, the plerixafor is administered to the donor at a dose of about 160 μg/kg. In various embodiments, the donor has renal dysfunction. In various embodiments, the donor has mild or moderate reduction in glomerular filtration rate (GFR). In various embodiments, the plerixafor is administered to the donor at a dose of about 240 μg/kg.

In various embodiments, the CXCR2 agonist is administered simultaneously with the CXCR4 antagonist. In various embodiments, the CXCR2 agonist is administered after the CXCR4 antagonist. In various embodiments, the CXCR2 agonist is administered within about 4 hours of administration of the CXCR4 antagonist. In various embodiments, the CXCR2 agonist is administered about 2 hours after the CXCR4 antagonist. In various embodiments, the CXCR2 agonist and the CXCR4 antagonist are each administered on two consecutive days. In various embodiments, the CXCR2 agonist and the CXCR4 antagonist are each administered once per day on two consecutive days.

Additionally disclosed herein is a method of obtaining hematopoietic stem or progenitor cells, the method comprising mobilizing a population of hematopoietic stem or progenitor cells according to the method described above and obtaining peripheral blood from the donor who has been diagnosed with multiple myeloma. In various embodiments, the peripheral blood is obtained between about 15 minutes and about 10 hours after administration of the CXCR2 agonist. In various embodiments, the peripheral blood is obtained between about 30 minutes and about 1 hour after administration of the CXCR2 agonist. In various embodiments, the peripheral blood is obtained about 30 minutes after administration of the CXCR2 agonist.

Additionally disclosed herein is a method of performing apheresis on the peripheral blood of a donor who has been diagnosed with multiple myeloma to produce an apheresis product, wherein the donor has been treated according to methods of mobilizing a population of hematopoietic stem or progenitor cells described herein. In various embodiments, about 10 L to about 30 L of peripheral blood is processed. In various embodiments, apheresis occurs over a period of time of from about 3 hours to about 5 hours. In various embodiments, the apheresis product has a volume of about 20 to about 400 mL.

In various embodiments, CD34+ cells are present in the apheresis product in an amount of at least about 2×10⁶ cells/kg. In various embodiments, CD34+ cells are present in the apheresis product in an amount of at least about 4×10⁶ cells/kg. In various embodiments, CD34+ cells are present in the apheresis product in an amount of at least about 6×10⁶ cells/kg. In various embodiments, CD34+ cells are present in the apheresis product in an amount of from about 1×10⁶ cells/kg to about 6×10⁶ cells/kg. In various embodiments, CD34+ cells are present in the apheresis product in an amount of from about 100×10⁶ cells to about 600×10⁶ cells. In various embodiments, the CD34+ cells are viable CD34+ cells.

In various embodiments, CD34⁺CD90⁺CD45RA⁻ cells are present in the apheresis product in an increased amount as compared to CD34⁺CD90⁺CD45RA⁻ cells mobilized by G-CSF. In various embodiments, CD34⁺CD90⁺CD45RA⁻ cells are at a frequency of about 15 to about 75% of CD34+ cells present in the apheresis product. In various embodiments, CD34⁺CD90⁺CD45RA⁻ cells are at a frequency of about 25 to about 55% of CD34+ cells present in the apheresis product. In various embodiments, CD34⁺CD90⁺CD45RA⁻ cells are at a frequency of about 30 to about 40% of CD34+ cells present in the apheresis product. In various embodiments, CD34⁺CD90⁺CD45RA⁻ cells are at a frequency of about 30 to about 35% of CD34+ cells present in the apheresis product. In various embodiments, CD34⁺CD90⁺CD45RA⁻ cells are present in the apheresis product in an amount of from about 0.1×10⁶ cells/kg to about 5×10⁶ cells/kg. In various embodiments, the CD34⁺CD90⁺CD45RA⁻ cells are viable CD34⁺CD90⁺CD45RA⁻ cells.

Additionally disclosed herein is a population of hematopoietic stem or progenitor cells, wherein the population of hematopoietic stem or progenitor cells is produced using the method of mobilizing a population of hematopoietic stem or progenitor cells or the method of obtaining hematopoietic stem or progenitor cells described herein. Additionally disclosed herein is a population of hematopoietic stem or progenitor cells, the population comprising between about 15 and 30 CD34⁺CD90⁺CD45RA⁻ cells per μL. In various embodiments, the population comprises between about 3 and about 15 CD34⁺CD90⁺CD45RA⁻ cells per μL. In various embodiments, the population comprises between about 10 and about 15 CD34⁺CD90⁺CD45RA⁻ cells per μL. In various embodiments, the population of hemopoietic stem or progenitor cells further comprise DMSO or citrate.

Additionally disclosed herein is an apheresis product isolated from a donor comprising CD34⁺CD90⁺CD45RA⁻ cells in an amount of from about 0.1×10⁶ cells/kg to about 5×10⁶ cells/kg or at a frequency of about 15 to about 75% of CD34+ cells present in the apheresis product, wherein the donor was previously diagnosed with multiple myeloma. In various embodiments, the CD34⁺CD90⁺CD45RA⁻ cells are viable CD34⁺CD90⁺CD45RA⁻ cells. In various embodiments, CD34+ cells are present in an amount of from about 1×10⁶ cells/kg to about 6×10⁶ cells/kg. In various embodiments, CD34+ cells are present in the apheresis product in an amount of from about 100×10⁶ cells to about 600×10⁶ cells. In various embodiments, the CD34+ cells are viable CD34+ cells. In various embodiments, CD34⁺CD90⁺CD45RA⁻ cells are at a frequency of about 25 to about 55% of CD34+ cells present in the apheresis product. In various embodiments, CD34⁺CD90⁺CD45RA⁻ cells are at a frequency of about 30 to about 40% of CD34+ cells present in the apheresis product. In various embodiments, CD34⁺CD90⁺CD45RA⁻ cells are at a frequency of about 30 to about 35% of CD34+ cells present in the apheresis product. In various embodiments, the concentration of white blood cells is higher in the apheresis product than in the peripheral blood of the donor.

In various embodiments, the apheresis product further comprises an anticoagulant. In various embodiments, the anticoagulant is citrate in an amount above physiological levels. In various embodiments, the anticoagulant is heparin. In various embodiments, the volume of the product is from about 20 to about 400 mL. In various embodiments, the apheresis product prevents, reduces the risk of developing, or reduces the severity of graft versus host disease (GVHD) in a patient in need thereof as compared to an apheresis product obtained from a donor administered G-CSF, where the apheresis product obtained from the donor administered G-CSF comprises hematopoietic stem cells that were mobilized into the peripheral blood of the donor following administration to the donor of a therapeutically effective amount of G-CSF.

Additionally disclosed herein is a method of treating multiple myeloma in a patient, the method comprising administering a population of hemopoietic stem or progenitor cells or an apheresis product described herein. In various embodiments, administering the population of hemopoietic stem or progenitor cells or the apheresis product comprises administering at least 3×10⁶ CD34+ cells/kg to the patient. In various embodiments, administering the population of hemopoietic stem or progenitor cells or the apheresis product comprises administering at least 4×10⁶ CD34+ cells/kg to the patient.

In various embodiments, the method of treating multiple myeloma in a patient further comprises administering melphalan to the patient. In various embodiments, administering melphalan to the patient comprises administering about 100 mg/m² to about 200 mg/m² of melphalan to the patient. In various embodiments, administering melphalan to the patient comprises administering about 140 mg/m² to about 200 mg/m² of melphalan to the patient. In various embodiments, administering melphalan to the patient comprises administering about 200 mg/m² of melphalan to the patient. In various embodiments, the donor with multiple myeloma and the patient with multiple myeloma is a single individual. In various embodiments, the population of hemopoietic stem or progenitor cells has an increased engraftment rate as compared to hemopoietic stem or progenitor cells mobilized by G-CSF. In various embodiments, increased engraftment rate is one of increased engraftment rate of neutrophils or increased engraftment rate of platelets in comparison to a proportion of patients receiving hematopoietic stem cells or progenitor cells that were mobilized into peripheral blood of a donor following administration to the donor of a therapeutically effective amount of G-CSF. In various embodiments, the increased engraftment rate is a neutrophil recovery that occurs within about 11-13 days. In various embodiments, the increased engraftment rate is a platelet recovery that occurs within about 16-19 days.

In various embodiments, the patient previously underwent induction therapy. In various embodiments, the patient previously underwent induction therapy for about 3 to about 6 months. In various embodiments, the induction therapy comprises a CyBorD regimen (cyclophosphamide (Cytoxan®, Procytox®), bortezomib (Velcade®) and dexamethasone (Decadron®, Dexasone®)); a VRD regimen (bortezomib, lenalidomide (Revlimid®) and dexamethasone; thalidomide (Thalomid®) and dexamethasone; lenalidomide and low-dose dexamethasone; bortezomib and dexamethasone; a VTD regimen (bortezomib, thalidomide and dexamethasone); bortezomib, cyclophosphamide and prednisone; bortezomib, doxorubicin (Adriamycin®) and dexamethasone; dexamethasone, or liposomal doxorubicin (Caelyx®, Doxil®), vincristine (Oncovin®) and dexamethasone. In various embodiments, the induction therapy comprises VTD. In various embodiments, the induction therapy further comprises daratumumab. In various embodiments, the patient has received about 4-6 cycles of lenalidomide. In various embodiments, the patient has stage I, stage II, or stage III multiple myeloma. In various embodiments, the patient has high risk fluorescence in situ hybridization (FISH). In various embodiments, the patient (e.g., the multiple myeloma patient) has at least one risk factor for poor mobilization. In various embodiments, the risk factor is a prior other cancer treated with chemotherapy and/or radiation, an autoimmune disorder needing systemic therapy, treatment with daratumumab, or alkylator exposure for more than 2 months.

Additionally disclosed herein is a method of mobilizing a population of hematopoietic stem or progenitor cells from the bone marrow of a mammalian donor into peripheral blood, the method comprising administering to the donor a CXCR2 agonist selected from the group consisting of Gro-β, Gro-β T, and variants thereof at a dose of from about 0.001 mg/kg to about 0.1 mg/kg, wherein the CXCR2 agonist is administered over a period of time of less than about 20 minutes such that the donor experiences fewer or less severe adverse effects in comparison to a donor who receives administration of the CXCR2 agonist over a period of time greater than about 20 minutes. In various embodiments, the dose is from greater than about 0.015 mg/kg to less than about 0.05 mg/kg. In various embodiments, the CXCR2 agonist comprises Gro-β T. In various embodiments, the CXCR2 agonist is administered at a dose of about 0.03 mg/kg. In various embodiments, the CXCR2 agonist is administered intravenously.

In various embodiments, methods disclosed herein further comprise administering to the donor a CXCR4 antagonist. In various embodiments, the CXCR4 antagonist is plerixafor. In various embodiments, the plerixafor is administered to the donor at a dose of about 160 μg/kg. In various embodiments, the plerixafor is administered to the donor at a dose of about 240 μg/kg. In various embodiments, the CXCR2 agonist is administered simultaneously with the CXCR4 antagonist. In various embodiments, the CXCR2 agonist is administered after the CXCR4 antagonist. In various embodiments, the CXCR2 agonist is administered within about 4 hours of administration of the CXCR4 antagonist. In various embodiments, the CXCR2 agonist is administered about 2 hours after the CXCR4 antagonist. In various embodiments, the CXCR2 agonist and the CXCR4 antagonist are each administered on two consecutive days. In various embodiments, the CXCR2 agonist and the CXCR4 antagonist are each administered once per day on two consecutive days.

Additionally disclosed herein is a method of obtaining hematopoietic stem or progenitor cells, the method comprising mobilizing a population of hematopoietic stem or progenitor cells according to methods disclosed herein, and obtaining peripheral blood from the donor. In various embodiments, the peripheral blood is obtained between about 15 minutes and about 10 hours after administration of the CXCR2 agonist. In various embodiments, the peripheral blood is obtained between about 30 minutes and about 1 hour after administration of the CXCR2 agonist. In various embodiments, the peripheral blood is obtained about 30 minutes after administration of the CXCR2 agonist.

Additionally disclosed herein is a method of performing apheresis on the peripheral blood of a donor to produce an apheresis product, wherein the donor has been treated according to the methods disclosed herein. In various embodiments, about 10 L to about 30 L of peripheral blood is processed. In various embodiments, apheresis occurs over a period of time of from about 3 hours to about 5 hours. In various embodiments, the apheresis product has a volume of about 20 to about 400 mL. In various embodiments, CD34+ cells are present in the apheresis product in an amount of at least about 2×10⁶ cells/kg. In various embodiments, CD34+ cells are present in the apheresis product in an amount of at least about 4×10⁶ cells/kg. In various embodiments, CD34+ cells are present in the apheresis product in an amount of at least about 6×10⁶ cells/kg. In various embodiments, CD34+ cells are present in the apheresis product in an amount of from about 1×10⁶ cells/kg to about 6×10⁶ cells/kg. In various embodiments, CD34+ cells are present in the apheresis product in an amount of from about 100×10⁶ cells to about 600×10⁶ cells. In various embodiments, the CD34+ cells are viable CD34+ cells.

In various embodiments, CD34⁺CD90⁺CD45RA⁻ cells are present in the apheresis product in an increased amount as compared to CD34⁺CD90⁺CD45RA⁻ cells mobilized by G-CSF. In various embodiments, CD34⁺CD90⁺CD45RA⁻ cells are at a frequency of about 15 to about 75% of CD34+ cells present in the apheresis product. In various embodiments, CD34⁺CD90⁺CD45RA⁻ cells are at a frequency of about 25 to about 55% of CD34+ cells present in the apheresis product. In various embodiments, CD34⁺CD90⁺CD45RA⁻ cells are at a frequency of about 30 to about 40% of CD34+ cells present in the apheresis product. In various embodiments, CD34⁺CD90⁺CD45RA⁻ cells are at a frequency of about 30 to about 35% of CD34+ cells present in the apheresis product. In various embodiments, CD34⁺CD90⁺CD45RA⁻ cells are present in the apheresis product in an amount of from about 0.1×10⁶ cells/kg to about 5×10⁶ cells/kg. In various embodiments, the CD34⁺CD90⁺CD45RA⁻ cells are viable CD34⁺CD90⁺CD45RA⁻ cells.

Additionally disclosed herein is a population of hematopoietic stem or progenitor cells, wherein the population of hematopoietic stem or progenitor cells is produced using the methods disclosed herein. In various embodiments, the population comprises between about 15 and 30 CD34⁺CD90⁺CD45RA⁻ cells per μL. In various embodiments, the population comprises between about 3 and about 15 CD34⁺CD90⁺CD45RA⁻ cells per μL. In various embodiments, the population comprises between about 10 and about 15 CD34⁺CD90⁺CD45RA⁻ cells per μL.

### BRIEF DESCRIPTION OF THE FIGURES

FIG. 1A depicts the treatments provided to patient cohorts enrolled in Part A and Part B of a Phase 1 clinical trial for MGTA-145 (Gro-β T) as well as measurable endpoints.

FIG. 1B depicts the treatments provided to patients enrolled in Part C and Part D of a Phase 1 clinical trial for MGTA-145 (Gro-β T).

FIG. 2A is a graph showing plasma concentrations of MGTA-145 following single dose administration (0.0075-0.3 mg/kg) as monotherapy in healthy subjects. Data are expressed as mean+/−SEM.

FIG. 2B is a graph showing plasma concentrations of MGTA-145 following single dose administration (0.03-0.15 mg/kg) in combination with a single dose of plerixafor (0.24 mg/kg) in healthy subjects. Data represent at least 4 subjects per dose level and are expressed as mean+/−SEM.

FIG. 3A is a graph showing the mobilization of CD34+ cells over the course of 24 hours following MGTA-145 monotherapy. FIG. 3B is a graph showing the fold change of CD34+ cells over the course of 24 hours following MGTA-145 monotherapy.

FIG. 4A is a graph showing the mobilization of CD34⁺CD90⁺CD45RA⁻ cells over the course of 24 hours following MGTA-145 monotherapy. FIG. 4B is a graph showing the fold change of CD34⁺CD90⁺CD45RA⁻ cells over the course of 24 hours following MGTA-145 monotherapy.

FIGS. 5A and 5B are graphs showing the mobilization of WBCs and neutrophils, respectively, over the course of 24 hours following MGTA-145 administration. The shaded region represents the normal reference range for healthy subjects.

FIGS. 6A and 6B are graphs showing the plasma levels of MMP-9 and molar ratio of MMP-9:TIMP-1, respectively, over the course of 24 hours following MGTA-145 monotherapy.

FIG. 7A is a graph showing the limited change in neutrophil activation markers (CD11b and CD18) following MGTA-145 monotherapy.

FIG. 7B is a graph further showing the limited change in neutrophil activation markers (L-selectin, CD11b, CD18, and CD66) following MGTA-145 monotherapy. Bars from left to right for each marker are placebo, 0.0075 mg/kg, 0.015 mg/kg, 0.03 mg/kg, 0.075 mg/kg, 0.15 mg/kg, and 0.3 mg/kg, respectively.

FIG. 8 is a graph showing that MGTA-145 monotherapy leads to rapid downregulation of its target receptor, CXCR2, on peripheral blood neutrophils, followed by recovery over 24 hours. Lines from bottom to top at 6 hrs post-administration represent 0.3 mg/kg, 0.15 mg/kg, 0.075 mg/kg, 0.03 mg/kg, 0.015 mg/kg, 0.075 mg/kg, and placebo, respectively.

FIG. 9A is a graph showing the mobilization of CD34⁺ cells over the course of 24 hours following simultaneous combination treatment of MGTA-145 and plerixafor versus plerixafor alone. FIG. 9B is a graph showing the fold change of CD34+ cells over the course of 24 hours following simultaneous combination treatment of MGTA-145 and plerixafor versus plerixafor alone.

FIG. 10 is a graph showing the mobilization of CD34⁺CD90⁺CD45RA⁻ cells over the course of 24 hours following MGTA-145+plerixafor therapy.

FIGS. 11A and 11B are graphs showing the mobilization of WBCs and neutrophils, respectively, over the course of 24 hours following simultaneous treatment of MGTA-145 and plerixafor.

FIG. 12 is a graph showing the limited change in neutrophil activation markers (CD11b and CD18) following MGTA-145+plerixafor therapy.

FIG. 13A is a graph showing the mobilization of CD34+ cells in response to a staggered combination therapy (MGTA-145 given two hours after plerixafor) at three doses of MGTA-145.

FIG. 13B is a graph showing the mobilization of CD34⁺CD90⁺CD45RA⁻ cells in response to a staggered combination therapy at three doses of MGTA-145.

FIG. 13C is a graph showing the percentage of CD34+ cells that are CD34⁺CD90⁺CD45RA⁻ cells following staggered combination therapy at two doses of MGTA-145.

FIG. 14A is a graph showing a comparison of the mobilization of CD34+ cells in response to either a simultaneous combination therapy or a staggered combination therapy.

FIG. 14B is a graph showing a comparison of the mobilization of CD34⁺CD90⁺CD45RA⁻ cells in response to either a simultaneous combination therapy or a staggered combination therapy.

FIGS. 15A and 15B are graphs showing the mobilization of WBCs and neutrophils, respectively, over the course of 24 hours following staggered combination treatment of MGTA-145 and plerixafor.

FIGS. 16A and 16B are graphs showing that staggered administration of 0.03 mg/kg or 0.07 mg/kg, respectively, MGTA-145 and plerixafor on two consecutive days led to the mobilization of CD34⁺ on both days, with fewer CD34⁺ cells mobilized on the second day (compare day 1 graph (Part B data) to day 2 graph (Part C data)).

FIG. 17A is a graph showing that staggered administration of 0.03 mg/kg MGTA-145 and plerixafor on two consecutive days led to the mobilization of neutrophils on both days, with fewer neutrophils mobilized on the second day. Similar results were found for administration of 0.07 mg/kg MGTA-145 and plerixafor (FIG. 17B).

FIG. 18 provides a graph showing that CXCR2 expression recovers to ˜80% of baseline prior to the second dose of MGTA-145 (0.07 mg/kg, staggered dosing).

FIG. 19 provides a graph showing that MGTA-145+plerixafor mobilizes 3-fold higher numbers of CD90+ cells than does G-CSF.

FIG. 20A provides graphs showing, from left to right, the collection yield of CD34+ cells following mobilization by MGTA-145+plerixafor or G-CSF, the frequency of CD34+CD90+CD45RA+ cells following mobilization by MGTA-145+plerixafor or G-CSF, and collection yield of CD34+CD90+CD45RA+ cells following mobilization by MGTA-145+plerixafor or G-CSF.

FIG. 20B provides graphs showing, from left to right, the collection yield of CD34+ cells following mobilization by MGTA-145 (at 0.03 mg/kg and 0.015 mg/kg)+plerixafor or G-CSF, the frequency of CD34+CD90+CD45RA+ cells following mobilization by MGTA-145+plerixafor or G-CSF, and collection yield of CD34+CD90+CD45RA+ cells following mobilization by MGTA-145+plerixafor or G-CSF.

FIG. 21A depicts a representative gating scheme for quantifying T cells.

FIG. 21B depicts a representative gating scheme for quantifying B and NK cells.

FIG. 22 provides representative flow plots for mice transplanted with MGTA-145+plerixafor-mobilized CD34+ cells.

FIG. 23A provides a graph showing SCID-repopulating cell (SRC) number per 1×10⁶ cells as determined by ELDA at week 4. FIG. 23B provides a graph showing SCID-repopulating cell (SRC) number per 1×10⁶ cells as determined by ELDA at week 12. FIG. 23C provides a graph showing SCID-repopulating cell (SRC) number per 1×10⁶ cells as determined by ELDA at week 16. Data are expressed as SRC number±95% CI. FIG. 23D shows a Kaplan-Meier survival curve of NSG mice transplanted with 6×10⁶ PBMCs from unmobilized healthy donors (n=3 donors, n=6-7 mice per donor) or healthy donors mobilized with MGTA-145+plerixafor, G-CSF, or plerixafor alone (n=3-6 donors per regimen, n=8 mice per donor). Data show significantly enhanced survival after transplant with MGTA-145+plerixafor. Statistics were determined by a log-rank test.

FIG. 24 provides a scheme outlining the experiment of Example 8 (left two panels). The right panel of FIG. 24 shows relative numbers of long-term HSC (LT-HSC) cells mobilized according to the various mobilization methods tested.

FIG. 25 provides bar graphs showing that transplantation with cells mobilized with MGTA-145+plerixafor led to higher relative engraftment (CRU) compared to transplantation with cells mobilized by any of the other mobilizing regimens.

FIG. 26 provides a representative gating scheme for evaluation of MGTA-145/plerixafor-mobilized blood.

FIG. 27 provides a representative gating scheme showing high editing in B2M gRNA+Cas9 groups (both DMSO cultures and AHR cultures).

FIG. 28A-F provides bar graphs showing that MGTA-145/plerixafor-mobilized blood can be edited by CRISPR-Cas9 and expanded by AHR. Specifically, as shown in FIGS. 28B and C, addition of AHR increases the numbers of CD34+ cells (FIG. 28B) and CD34+CD90+CD45RA− cells (FIG. 28C) under all conditions (mock, mock pulse, and B2M), as compared to control TNC cells (FIG. 28A). Further, as shown in FIGS. 28D-F, TNC cells (FIG. 28D), CD34+ cells (FIG. 28E) and CD34⁺CD90⁺CD45RA⁻ cells (FIG. 28F) were all edited by CRISPR-Cas9 under the conditions tested.

FIG. 29 provides a bar graph showing that a 7-day culturing protocol with AHR results in a 15-fold expansion of CD34+ cells over the typical 2-day culturing protocol typically used for CRISPR-Cas9 editing (i.e., 1 day pre-stimulation prior to electroporation with a gRNA and Cas9, followed by a 1 day post-EP culture.

FIG. 30 provides a bar graph showing that there is no difference in editing rates between G-CSF-mobilized CD34+ cells and MGTA-145/plerixafor-mobilized CD34+ cells.

FIGS. 31A-D provides bar graphs showing that CD34+ cells mobilized from a human donor and subjected to gene modification show similar numbers of CD34⁺CD90⁺CD45RA⁻ cells before and after gene modification and that a >80% editing rate was achieved (FIG. 31A). Further, gene edited cells are capable of engraftment at a similar rate as mock-edited cells and that the editing rate of the engrafted cells remains >80% (FIGS. 31B-C). FIG. 31D shows a engraftment in NSG mice of peripheral blood cells mobilized with MGTA-145+plerixafor and subsequently gene modified (edited) and then expanded using the AHR antagonist E478. Expansion using E478 led to a higher percentage of engraftment as compared to cells that were not expanded. The right-hand panel shows that use of E478 does not affect editing rate.

FIGS. 32A and 32B depict survival of mice transplanted with CD14 depleted cells from two different donors.

FIG. 33 depicts the per apheresis yield and total stem cell yields across 15 multiple myeloma patients.

FIG. 34 depicts the per apheresis yield and total stem cell yields across 15 multiple myeloma patients as well as 10 multiple myeloma patients included in the clinical trial expansion cohort.

### DETAILED DESCRIPTION

The present invention provides compositions and methods for mobilizing hematopoietic stem and progenitor cells in a subject. For example, the subject may be a hematopoietic stem and progenitor cell donor (i.e., a donor), such as a mammalian donor (e.g., a human donor). The compositions and methods described herein can additionally be used for the treatment of one or more stem cell disorders in a patient, such as a human patient. In various embodiments, the donor and the patient are the same individual. In various embodiments, the donor and the patient are the same individual who is previously diagnosed with multiple myeloma. Therefore, hematopoietic stem and progenitor cells are mobilized in the donor with multiple myeloma, and provided through an autologous transplant to the same donor.

Using the compositions and methods described herein, a C-X-C chemokine receptor type 2 (CXCR2) agonist, such as Gro-β or a variant thereof, such as a truncated form of Gro-β (e.g., Gro-β T), as described herein, optionally in combination with a C-X-C chemokine receptor type 4 (CXCR4) antagonist, such as 1,1′-[1,4-phenylenebis(methylene)]-bis-1,4,8,11-tetra-azacyclotetradecane or a variant thereof, may be administered to a donor, as described herein, in amounts sufficient to mobilize hematopoietic stem and progenitor cells. The compositions and methods described herein thus enable the selective mobilization of hematopoietic stem and progenitor cells in a donor, which may then be isolated from a donor for therapeutic use.

The invention is based, in part, on the discovery that administration of a surprisingly low dose of a CXCR2 agonist, such as Gro-β, Gro-β T, or a variant thereof, optionally in combination with a CXCR4 antagonist, such as plerixafor or a pharmaceutically acceptable salt thereof, at particular doses can provide the important clinical benefit of mobilizing hematopoietic stem and progenitor cells. In addition, CD34⁺CD90⁺CD45⁻ cells, a population indicative of a stem cell phenotype associated with long term engraftment, are effectively mobilized by the methods of administration as described herein. Thus, the populations of mobilized hematopoietic stem and progenitor cells produced using the compositions and methods described herein are particularly suitable for hematopoietic stem cell transplantation therapy.

Following mobilization, the hematopoietic stem or progenitor cells may be isolated for ex vivo expansion and/or for therapeutic use. In some embodiments, upon collection of the mobilized hematopoietic stem and/or progenitor cells, the withdrawn cells may be infused into a patient, such as the donor or another subject (e.g., a subject that is HLA-matched to the donor) for the treatment of one or more pathologies of the hematopoietic system. Additionally or alternatively, the mobilized cells may be withdrawn and then expanded ex vivo, such as by contacting the cells with an aryl hydrocarbon receptor antagonist, so as to produce a population of hematopoietic stem cells having a sufficient quantity of cells for transplantation.

As described herein, hematopoietic stem cells are capable of differentiating into a multitude of cell types in the hematopoietic lineage, and can thus be administered to a patient in order to populate or repopulate a cell type that is defective or deficient in the patient. The patient may be one, for example, that is suffering from one or more blood disorders, such as an autoimmune disease, cancer, hemoglobinopathy, or other hematopoietic pathology, and is therefore in need of hematopoietic stem cell transplantation. The invention thus provides methods of treating a variety of hematopoietic conditions, such as sickle cell anemia, thalassemia, Fanconi anemia, Wiskott-Aldrich syndrome, adenosine deaminase deficiency-severe combined immunodeficiency, metachromatic leukodystrophy, Diamond-Blackfan anemia and Schwachman-Diamond syndrome, human immunodeficiency virus infection, and acquired immune deficiency syndrome, as well as cancers and autoimmune diseases, among others.

The sections that follow provide a description of CXCR4 antagonists and CXCR2 agonists that can be administered to a donor so as to induce mobilization of a population of hematopoietic stem or progenitor cells from a stem cell niche into peripheral blood, from which the cells may subsequently be isolated and infused into a patient for the treatment, for example, of one or more stem cell disorders, such as a cancer, autoimmune disease, of metabolic disorder described herein. The following sections additionally describe methods of determining whether populations of cells mobilized with a CXCR2 agonist and/or a CXCR4 antagonist are suitable for release for ex vivo expansion and/or for therapeutic applications.

### Definitions

As used herein, the term “about” refers to a value that is within 10% above or below the value being described. For example, the term “about 5 nM” indicates a range of from 4.5 nM to 5.5 nM.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)₂, Fab, Fv, rlgG, and scFv fragments. Unless otherwise indicated, the term “monoclonal antibody” (mAb) is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, i-body, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al. (1989) Nature 341:544-546); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al. (1988) Science 242:423-426 and Huston et al. (1988) Proc. Natl. Acad. Sci. USA 85:5879-5883). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “bispecific antibody” refers to, for example, a monoclonal, often a human or humanized antibody that is capable of binding at least two different antigens or two different epitopes on the same antigen.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a n-sheet configuration, connected by three CDRs, which form loops that connect, and in some cases form part of, the n-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, MD., 1987). As used herein, numbering of immunoglobulin amino acid residues is performed according to the immunoglobulin amino acid residue numbering system of Kabat et al., unless otherwise indicated.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in TABLE 1 below.

TABLE 1
Representative physicochemical properties
of naturally-occurring amino acids

| | | | | Electrostatic | |
|---|---|---|---|---|---|
| | 3 | 1 | | character at | |
| | Letter | Letter | Side-chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A3: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, “CRU (competitive repopulating unit)” refers to a unit of measure of long-term engrafting stem cells, which can be detected after in-vivo transplantation.

As used herein, the term “donor” refers to a subject, such as a mammalian subject (e.g., a human subject) from which one or more cells are isolated prior to administration of the cells, or progeny thereof, into a recipient, or, with respect to neutropenia, the term “donor” is used to mean “subject” or “patient,” because in the context of treating neutropenia, cells are not isolated from the subject or patient and donated to a recipient. The one or more cells may be, for example, a population of hematopoietic stem or progenitor cells.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al. (1993) Proc. Natl. Acad. Sci. USA 90:6444-48).

As used herein, the term “disrupt” with respect to a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying hematopoietic stem/progenitor cells are detailed in U.S. Pat. No. 8,518,701; U.S. 2010/0251395; and U.S. 2012/0222143, the disclosures of each of which are incorporated herein by reference in their entirety (in case of conflict, the instant specification is controlling).

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA, 82:6148-6152), gene targeting into embryonic stem cells (Thompson et al. (1989) Cell, 56:313-321), electroporation of embryos (Lo (1983)Mol. Cell. Biol., 3:1803-1814), sperm-mediated gene transfer (Lavitrano et al. (2002) Proc. Natl. Acad. Sci. USA, 99:14230-14235; Lavitrano et al. (2006) Reprod. Fert. Develop., 18:19-23), and in vitro transformation of somatic cells, such as cumulus or mammary cells, or adult, fetal, or embryonic stem cells, followed by nuclear transplantation (Wilmut et al. (1997) Nature, 385:810-813; and Wakayama et al. (1998) Nature, 394:369-374). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for example, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

As used herein, a “dual variable domain immunoglobulin” (“DVD-Ig”) refers to an antibody that combines the target-binding variable domains of two monoclonal antibodies via linkers to create a tetravalent, dual-targeting single agent (see, for example, Gu et al. (2012) Meth. Enzymol., 502:25-41).

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

As used herein, the term “hematopoietic progenitor cells” includes pluripotent cells capable of differentiating into several cell types of the hematopoietic system, including, without limitation, granulocytes, monocytes, erythrocytes, megakaryocytes, B-cells and T-cells, among others. Hematopoietic progenitor cells are committed to the hematopoietic cell lineage and generally do not self-renew. Hematopoietic progenitor cells can be identified, for example, by expression patterns of cell surface antigens, and include cells having the following immunophenotype: Lin⁻KLS⁺Flk2⁻CD34⁺. Hematopoietic progenitor cells include short-term hematopoietic stem cells, multi-potent progenitor cells, common myeloid progenitor cells, granulocyte-monocyte progenitor cells, and megakaryocyte-erythrocyte progenitor cells. The presence of hematopoietic progenitor cells can be determined functionally, for example, by detecting colony-forming unit cells, e.g., in complete methylcellulose assays, or phenotypically through the detection of cell surface markers using flow cytometry and cell sorting assays described herein and known in the art.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker. In humans, CD34⁺ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34⁺, CD38⁻, CD45RA⁻, CD90⁺, CD49F⁺, and lin⁻ (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1⁺, C-kit⁺, CD135⁻, Slamfl/CD150⁺, and lin⁻ (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the terms “Major histocompatibility complex antigens” (“MHC”, also referred to as “human leukocyte antigens” (“HLA”) in the context of humans) refer to proteins expressed on the cell surface that confer a unique antigenic identity to a cell. MHC/HLA antigens are target molecules that are recognized by T cells and NK cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells. Both have been implicated in the rejection of transplanted organs. An important aspect of the HLA gene system is its polymorphism. Each gene, MHC class I (A, B and C) and MHC class II (DP, DQ and DR) exists in different alleles. For example, two unrelated individuals may carry class I HLA-B, genes B5, and Bw41, respectively. Allelic gene products differ in one or more amino acids in the α and/or β domain(s). Large panels of specific antibodies or nucleic acid reagents are used to type HLA haplotypes of individuals, using leukocytes that express class I and class II molecules. The genes commonly used for HLA typing are the six MHC Class I and Class II proteins, two alleles for each of HLA-A; HLA-B and HLA-DR. The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes”, i.e. the set of alleles present on a single chromosome, which is inherited from one parent, tend to be inherited as a group. The set of alleles inherited from each parent forms a haplotype, in which some alleles tend to be associated together. Identifying a patient's haplotypes can help predict the probability of finding matching donors and assist in developing a search strategy, because some alleles and haplotypes are more common than others and they are distributed at different frequencies in different racial and ethnic groups.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and are thus less likely to mount an immune response against the transplant.

As used herein, the term “HLA-mismatched” refers to a donor-recipient pair in which at least one HLA antigen, in particular with respect to HLA-A, HLA-B and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the term “human antibody” refers to an antibody in which substantially every part of the protein (for example, all CDRs, framework regions, C_{L}, C_{H} domains (e.g., CHI, C_{H}2, C_{H}3), hinge, and V_{L} and V_{H} domains) is substantially non-immunogenic in humans, with only minor sequence changes or variations. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

As used herein, the term “humanized” antibody refers to a non-human antibody that contains minimal sequences derived from non-human immunoglobulin. In general, a humanized antibody contains substantially all of at least one, and typically two, variable domains, in which all or substantially all of the CDR regions correspond to those of a non-human immunoglobulin. All or substantially all of the FW regions may also be those of a human immunoglobulin sequence. The humanized antibody can also contain at least a portion of an immunoglobulin constant region (Fc), typically that of a human immunoglobulin consensus sequence. Methods of antibody humanization are known in the art and have been described, for example, in Riechmann et al. (1988) Nature 332:323-7; U.S. Pat. Nos. 5,530,101; 5,585,089; 5,693,761; 5,693,762; and 6,180,370.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. In some embodiments, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher Disease, Hurler Disease, sphingolipidoses, metachromatic leukodystrophy, globoid cell leukodystrophy, cerebral adrenoleukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for example, by way of flow cytometry and fluorescence activated cell sorting (FACS) methods, among other procedures, known in the art.

As used herein, the term “leukocyte” refers to a heterogeneous group of nucleated blood cell types, and excludes erythrocytes and platelets. Leukocytes can be divided into two general groups: polymorphonucleocytes, which include neutrophils, eosinophils, and basophils, and mononucleocytes, which include lymphocytes and monocytes. Polymorphonucleocytes contain many cytoplasmic granules and a multilobed nucleus and include the following: neutrophils, which are generally amoeboid in shape, phagocytic, and stain with both basic and acidic dyes, and eosinophils and basophils, which contain cytoplasmic granules that stain with acidic dyes and with basic dyes, respectively.

As used herein, the term “lymphocyte” refers to a mononuclear leukocyte that is involved in the mounting of an immune response. In general, lymphocytes include B lymphocytes, T lymphocytes, and NK cells.

As used herein, the terms “mobilize” and “mobilization” refer to processes by which a population of hematopoietic stem or progenitor cells (e.g., a neutrophil) is released from a stem cell niche, such as the bone marrow of a subject, into circulation in the peripheral blood. Mobilization of hematopoietic stem and progenitor cells can be monitored, for example, by assessing the quantity or concentration of hematopoietic stem or progenitor cells in a peripheral blood sample isolated from a subject. For example, the peripheral blood sample may be withdrawn from the subject, and the quantity or concentration of hematopoietic stem or progenitor cells in the peripheral blood sample may subsequently be assessed, following the administration of a hematopoietic stem or progenitor cell mobilization regimen to the subject. The mobilization regimen may include, for example, a CXCR4 antagonist, such as a CXCR4 antagonist described herein (e.g., plerixafor or a variant thereof), and a CXCR2 agonist, such as a CXCR2 agonist described herein (e.g., Gro-R or a variant thereof, such as a truncation of Gro-β, for example, Gro-β T). The quantity or concentration of hematopoietic stem or progenitor cells in the peripheral blood sample isolated from the subject following administration of the mobilization regimen may be compared to the quantity or concentration of hematopoietic stem or progenitor cells in a peripheral blood sample isolated from the subject prior to administration of the mobilization regimen. An observation that the quantity or concentration of hematopoietic stem or progenitor cells has increased in the peripheral blood of the subject following administration of the mobilization regimen is an indication that the subject is responding to the mobilization regimen, and that hematopoietic stem and progenitor cells have been released from one or more stem cell niches, such as the bone marrow, into peripheral blood circulation. In some embodiments, an observation that the quantity or concentration of hematopoietic stem or progenitor cells has increased in the peripheral blood of the subject by 1%, 100%, 1,000%, or more (e.g., by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1,000%, or more) following administration of the mobilization regimen is an indication that the subject is responding to the mobilization regimen, and that hematopoietic stem and progenitor cells have been released from one or more stem cell niches, such as the bone marrow, into peripheral blood circulation. Methods for determining the quantity or concentration of hematopoietic stem or progenitor cells are described herein and known in the art, and include, for example, flow cytometry techniques that quantify hematopoietic stem or progenitor cells on the basis of the antigen expression profile of such cells, which is described herein. For example, human HSCs are CD34⁺, CD38⁻, CD45RA⁻, CD90⁺, CD49F⁺, and lin− (negative for mature lineage markers including CD2, CD3, CD4, CD7, CD8, CD10, CD11B, CD19, CD20, CD56, CD235A). Additional methods for determining the quantity or concentration of hematopoietic stem or progenitor cells in a peripheral blood sample isolated from a subject include assays that quantify the number of colony-forming units (CFUs) in the sample, which is a measure of the quantity of viable hematopoietic stem or progenitor cells that, upon incubation with an appropriate culture medium, give rise to an individual population of hematopoietic stem or progenitor cells.

As used herein, the term “mobilizing amount” refers to a quantity of one or more agents, such as a quantity of a CXCR4 antagonist and/or a CXCR2 agonist described herein (In some embodiments, a quantity of plerixafor, or a variant thereof, and/or Gro-β, or a variant thereof, such as a truncation of Gro-β, for example, Gro-β T) that mobilizes a population of hematopoietic stem or progenitor cells upon administration to a subject, such as a mammalian subject (e.g., a human subject). Exemplary mobilizing amounts of these agents include amounts sufficient to effectuate the release of a population of, for example, from about 20 to about 40 CD34⁺ cells/μL of peripheral blood, such as from about 21 to about 39 CD34⁺ cells/μL of peripheral blood, about 22 to about 38 CD34⁺ cells/μL of peripheral blood, about 23 to about 37 CD34⁺ cells/μL of peripheral blood, about 24 to about 36 CD34+ cells/μL of peripheral blood, about 25 to about 35 CD34⁺ cells/μL of peripheral blood, about 26 to about 34 CD34⁺ cells/μL of peripheral blood, about 27 to about 33 CD34⁺ cells/μL of peripheral blood, about 28 to about 32 CD34⁺ cells/μL of peripheral blood, or about 29 to about 31 CD34⁺ cells/μL of peripheral blood (e.g., about 20 CD34⁺ cells/μL of peripheral blood, 21 CD34⁺ cells/μL of peripheral blood, 22 CD34⁺ cells/μL of peripheral blood, 23 CD34⁺ cells/μL of peripheral blood, 24, CD34⁺ cells/μL of peripheral blood, 25 CD34+ cells/μL of peripheral blood, 26 CD34⁺ cells/μL of peripheral blood, 27 CD34⁺ cells/μL of peripheral blood, 28 CD34⁺ cells/μL of peripheral blood, 29 CD34⁺ cells/μL of peripheral blood, 30 CD34⁺ cells/μL of peripheral blood, 31 CD34⁺ cells/μL of peripheral blood, 32 CD34⁺ cells/μL of peripheral blood 33 CD34⁺ cells/μL of peripheral blood, 34 CD34+ cells/μL of peripheral blood, 35 CD34⁺ cells/μL of peripheral blood, 36 CD34⁺ cells/μL of peripheral blood, 37 CD34⁺ cells/μL of peripheral blood, 38 CD34⁺ cells/μL of peripheral blood, 39 CD34⁺ cells/μL of peripheral blood, 40 CD34⁺ cells/μL of peripheral blood, or more. In certain embodiments, mobilizing amounts of these agents include amounts sufficient to effectuate the release of a population of, for example, from about 5 to about 20 CD34+CD90+CD45RA− cells/μL of peripheral blood, such as from about 5 to about 8 CD34+CD90+CD45RA− cells/μL of peripheral blood, about 5 to about 10 CD34+CD90+CD45RA− cells/μL of peripheral blood, about 5 to about 12 CD34+CD90+CD45RA− cells/μL of peripheral blood, about 5 to about 15 CD34+CD90+CD45RA− cells/μL of peripheral blood, about 5 to about 18 CD34+CD90+CD45RA− cells/μL of peripheral blood, about 8 to about 10 CD34+CD90+CD45RA− cells/μL of peripheral blood, about 8 to about 12 CD34+CD90+CD45RA− cells/μL of peripheral blood, about 8 to about 15 CD34+CD90+CD45RA− cells/μL of peripheral blood, or about 8 to about 18 CD34+CD90+CD45RA− cells/μL of peripheral blood, about 8 to about 20 CD34+CD90+CD45RA− cells/μL of peripheral blood, about 10 to about 12 CD34+CD90+CD45RA− cells/μL of peripheral blood, about 10 to about 15 CD34+CD90+CD45RA− cells/μL of peripheral blood, about 10 to about 18 CD34+CD90+CD45RA− cells/μL of peripheral blood, about 10 to about 20 CD34+CD90+CD45RA− cells/μL of peripheral blood, about 12 to about 15 CD34+CD90+CD45RA− cells/μL of peripheral blood, about 10 to about 18 CD34+CD90+CD45RA− cells/μL of peripheral blood, about 10 to about 20 CD34+CD90+CD45RA− cells/μL of peripheral blood, about 12 to about 15 CD34+CD90+CD45RA− cells/μL of peripheral blood, about 12 to about 18 CD34+CD90+CD45RA− cells/μL of peripheral blood, about 12 to about 20 CD34+CD90+CD45RA− cells/μL of peripheral blood, about 15 to about 18 CD34+CD90+CD45RA− cells/μL of peripheral blood, or about 15 to about 20 CD34+CD90+CD45RA− cells/μL of peripheral blood. In certain embodiments, mobilizing amounts of these agents include amounts sufficient to effectuate at least a 2 fold release of a population CD34+CD90+CD45RA− cells/μL of peripheral blood, e.g., at least a 3 fold release, at least a 4 fold release, at least a 5 fold release, at least a 6 fold release at least a 7 fold release, at least an 8 fold release, at least a 9 fold release or at least a 10 fold release of a population CD34+CD90+CD45RA− cells/μL of peripheral blood. In certain embodiments, mobilizing amounts of these agents include amounts sufficient to effectuate a 2 fold release to a 10 fold release, e.g., a 2 fold to 4 fold release, a 2 fold to 6 fold release, a 2 fold to 8 fold release, a 4 fold to 6 fold release, a 4 fold to 8 fold release, a 4 fold to 10 fold release, a 6 fold to 8 fold release, a 6 fold to 10 fold release, or a 8 fold to 10 release of a population CD34⁺CD90⁺CD45RA⁻ cells/μL of peripheral blood.

As used herein, the term “monoclonal antibody” refers to an antibody that is derived from a single clone, including any eukaryotic, prokaryotic, or phage clone, and not the method by which it is produced.

As used herein, the term “monocyte” refers to a CD14+ and CD34 peripheral blood mononuclear cell (PBMC), which is generally capable of differentiating into a macrophage and/or dendritic cell upon activation by one or more foreign substances, such as, a microbial product. In particular, a monocyte may express elevated levels of the CD14 surface antigen marker, and may express at least one biomarker selected from CD64, CD93, CD180, CD328 (also known as sialic acid-binding Ig-like lectin 7 or Siglec7), and CD329 (sialic acid-binding Ig-like lectin 9 or Siglec9), as well as the peanut agglutinin protein (PNA).

As used herein, a “peptide” refers to a single-chain polyamide containing a plurality of amino acid residues, such as naturally-occurring and/or non-natural amino acid residues, that are consecutively bound by amide bonds. Examples of peptides include shorter fragments of full-length proteins, such as full-length naturally-occurring proteins.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject. A sample may be, for example, withdrawn peripheral blood from a donor that is undergoing or has undergone a hematopoietic stem or progenitor cell mobilization regimen described herein.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

As used herein, the phrase “stem cell disorder” broadly refers to any disease, disorder, or condition that may be treated or cured by engrafting or transplanting a population of hematopoietic stem or progenitor cells in a target tissue within a patient. For example, Type I diabetes has been shown to be cured by hematopoietic stem cell transplant, along with various other disorders. Exemplary diseases that can be treated by infusion of hematopoietic stem or progenitor cells into a patient are sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated by transplantation of hematopoietic stem and progenitor cells as described herein include blood disorders (e.g., sickle cell anemia) and autoimmune disorders, such as scleroderma, multiple sclerosis, ulcerative colitis, and Crohn's disease. Additional diseases that may be treated using hematopoietic stem and progenitor cell transplant therapy include cancer, such as a cancer described herein. Exemplary stem cell disorders are malignancies, such as a neuroblastoma or a hematologic cancer, such as leukemia, lymphoma, and myeloma. In some embodiments, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using hematopoietic stem or progenitor cell transplant therapy include myelodysplastic syndrome. In some embodiments, the patient has or is otherwise affected by a metabolic storage disorder. For example, the patient may suffer or otherwise be affected by a metabolic disorder selected from the group consisting of glycogen storage diseases, mucopolysaccharidoses, Gaucher Disease, Hurler Disease, sphingolipidoses, metachromatic leukodystrophy, globoid cell leukodystrophy, cerebral adrenoleukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem or progenitor cell transplant therapy.

As used herein in the context of hematopoietic stem cell mobilization, the term “stem cell niche” refers to a microenvironment within a donor, such as a mammalian donor (e.g., a human donor) in which endogenous hematopoietic stem or progenitor cells reside. An exemplary stem cell niche is bone marrow tissue.

As used herein, the terms “subject” and “patient” refer to an organism, such as a human, that receives treatment for a particular disease or condition as described herein. In some embodiments, a patient, such as a human patient, that is in need of hematopoietic stem cell transplantation may receive treatment that includes a population of hematopoietic stem cells so as to treat a stem cell disorder, such as a cancer, autoimmune disease, or metabolic disorder described herein. The hematopoietic stem cells that are transplanted into the patient may be, for example, a population of hematopoietic stem cells that has been mobilized and withdrawn from a donor in accordance with the compositions and methods described herein. In some embodiments, the hematopoietic stem cells that are transplanted into the patient may be mobilized within a donor by administration of a CXCR4 antagonist and/or a CXCR2 agonist to the donor. In certain embodiments, the terms “subject” and “patient” refer to an organism, such as a human, that receives treatment for neutropenia.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder or to promote a beneficial phenotype in the patient being treated. Beneficial or desired clinical results include, but are not limited to, promoting the engraftment of exogenous hematopoietic cells in a patient following hematopoietic stem or progenitor cell transplant therapy. In certain embodiments, the benefits include a more rapid engraftment of transplanted cells, e.g., neutrophils and platelets. For example, in certain embodiments, using the methods described herein, neutrophil recovery occurs within about 5-20 days post-transplant, about 5-15 days post-transplant, about 5-10 days post-transplant, about 7-12 days post-transplant, about 8-12 days post-transplant, about 9-15 days post-transplant, about 10-15 days post-transplant, about 11-13 days post-transplant, or about 10 days post-transplant. In particular embodiments, using the methods described herein, neutrophil recovery occurs within about 11-13 days post-transplant.

In certain embodiments, using the methods described herein, platelet recovery occurs within about 10-20 days post-transplant, about 10-15 days post-transplant, about 15-20 days post-transplant, about 16-19 days post-transplant, about 12-18 days post-transplant, about 12-17 days post-transplant, about 13-18 days post-transplant, about 12-17 days post-transplant, or about 15 days post-transplant. In particular embodiments, using the methods described herein, platelet recovery occurs within about 16-19 days post-transplant.

Additional beneficial results include an increase in the cell count or relative concentration of hematopoietic stem cells in a patient in need of a hematopoietic stem or progenitor cell transplant following administration of an exogenous hematopoietic stem or progenitor cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following and subsequent hematopoietic stem cell transplant therapy. Additional beneficial results may include the reduction in quantity of a disease-causing cell population, such as a population of cancer cells or autoimmune cells. In the context of treating neutropenia, beneficial or desired clinical results include, but are not limited to, increasing the number of neutrophils in the blood and/or preventing or reducing at least one symptom associated with neutropenia.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, an RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of peptides and proteins, such as those described herein, include plasmids that contain regulatory sequences, such as promoter and enhancer regions, which direct gene transcription. Other useful vectors for expression of peptides and proteins described herein contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “alkyl” refers to a straight- or branched-chain alkyl group having, for example, from 1 to 20 carbon atoms in the chain. Examples of alkyl groups include methyl, ethyl, n-propyl, isopropyl, butyl, isobutyl, sec-butyl, tert-butyl, pentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain. The divalent positions may be one or more heteroatoms.

As used herein, the term “alkenyl” refers to a straight- or branched-chain alkenyl group having, for example, from 2 to 20 carbon atoms in the chain. Examples of alkenyl groups include vinyl, propenyl, isopropenyl, butenyl, tert-butylenyl, hexenyl, and the like.

As used herein, the term “alkenylene” refers to a straight- or branched-chain divalent alkenyl group. The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain. The divalent positions may be one or more heteroatoms.

As used herein, the term “alkynyl” refers to a straight- or branched-chain alkynyl group having, for example, from 2 to 20 carbon atoms in the chain. Examples of alkynyl groups include propargyl, butynyl, pentynyl, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain. The divalent positions may be one or more heteroatoms.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like.

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocycloalkyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, and the like. The divalent positions may be one or more heteroatoms.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group. Heteroaryl groups include pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heteroarylene” refers to a divalent heteroaryl group. The divalent positions may be on the same or different atoms. The divalent positions may be one or more heteroatoms.

Unless otherwise constrained by the definition of the individual substituent, the foregoing chemical moieties, such as “alkyl,” “alkylene,” “heteroalkyl,” “heteroalkylene,” “alkenyl,” “alkenylene,” “heteroalkenyl,” “heteroalkenylene,” “alkynyl,” “alkynylene,” “heteroalkynyl,” “heteroalkynylene,” “cycloalkyl,” “cycloalkylene,” “heterocyclolalkyl,” heterocycloalkylene,” “aryl,” “arylene,” “heteroaryl,” and “heteroarylene” groups can optionally be substituted. As used herein, the term “optionally substituted” refers to a compound or moiety containing one or more (for example, 1, 2, 3, 4, 5, 6, 7, 8, 9, 10, or more) substituents, as permitted by the valence of the compound or moiety or a site thereof, such as a substituent selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. The substitution may include situations in which neighboring substituents have undergone ring closure, such as ring closure of vicinal functional substituents, to form, for example, lactams, lactones, cyclic anhydrides, acetals, hemiacetals, thioacetals, aminals, and hemiaminals, formed by ring closure, for example, to furnish a protecting group.

### Methods of Mobilizing Hematopoietic Stem and Progenitor Cells and Releasing Cells for Expansion and Therapeutic Use

The present invention is based, in part, on the discovery that hematopoietic stem and progenitor cells (e.g., neutrophils) can be mobilized by administering particular doses of a CXCR2 agonist, such as Gro-β, Gro-β T, or a variant thereof, optionally in combination with a CXCR4 antagonist to a mammalian donor (e.g., a human donor).

### CXCR2 Agonists

### Gro-β, Gro-β T, and Variants Thereof

Exemplary CXCR2 agonists that may be used in conjunction with the compositions and methods described herein are Gro-β and variants thereof. Gro-β (also referred to as growth-regulated protein J, chemokine (C-X-C motif) ligand 2 (CXCL2), and macrophage inflammatory protein 2-α (MIP2-α)) is a cytokine capable of mobilizing hematopoietic stem and progenitor cells, for example, by stimulating the release of proteases, and particularly MMP-9, from peripheral neutrophils. In contrast, TIMP-1 blocks MMP migration. Without being limited by mechanism, MMP-9 may induce mobilization of hematopoietic stem and progenitor cells from stem cell niches, such as the bone marrow, to circulating peripheral blood by stimulating the degradation of proteins such as stem cell factor, its corresponding receptor, CD117, and CXCL12, all of which generally maintain hematopoietic stem and progenitor cells immobilized in bone marrow. Therefore, an increase in MMP-9 or an increase in MMP-9:TIMP-1 ratio can be indicative of increased mobilization of hematopoietic stem and progenitor cells and/or neutrophils.

Accordingly, in certain embodiments, therefore, the method results in an increase in plasma MMP-9 for example, to an amount of about 100 ng/mL to about 300 ng/mL (e.g. about 150 ng/mL to about 250 ng/mL) peripheral blood within 30 minutes of administration of a CXCR2 agonist (e.g., Gro-β or Gro-β T). In certain embodiments, the method results in an increase in the MMP-9:TIMP-1 ratio in peripheral blood, e.g., to a molar ratio of between about 0.2 and about 0.6 (e.g., about 0.25 to about 0.45) MMP-9:TIMP-1.

In addition to Gro-β, exemplary CXCR2 agonists that may be used in conjunction with the compositions and methods described herein are truncated forms of Gro-β, such as those that feature a deletion at the N-terminus of Gro-3 of from 1 to 8 amino acids (e.g. peptides that feature an N-terminal deletion of 1 amino acids, 2 amino acids, 3 amino acids, 4 amino acids, 5 amino acids, 6 amino acids, 7 amino acids, or 8 amino acids). In some embodiments, CXCR2 agonists that may be used in conjunction with the compositions and methods described herein include Gro-β T, which is characterized by a deletion of the first four amino acids from the N-terminus of Gro-β. Gro-β T exhibits particularly advantageous biological properties, such as the ability to induce hematopoietic stem and progenitor cell mobilization with a potency superior to that of Gro-β by multiple orders of magnitude. Gro-β and Gro-β T are described, for example, in U.S. Pat. No. 6,080,398, the disclosure of which is incorporated herein by reference in its entirety.

In addition, exemplary CXCR2 agonists that may be used in conjunction with the compositions and methods described herein are variants of Gro-β containing an aspartic acid residue in place of the asparagine residue at position 69 of SEQ ID NO: 1. This peptide, is referred to herein as Gro-β N69D. Similarly, CXCR2 agonists that may be used with the compositions and methods described herein include variants of Gro-β T containing an aspartic acid residue in place of the asparagine residue at position 65 of SEQ ID NO: 2. This peptide, referred to herein as Gro-β T N65D, not only retains hematopoietic stem and progenitor cell-mobilizing capacity, but exhibits a potency that is substantially greater than that of Gro-β T. Gro-β N69D and Gro-β T N65D are described, for example, in U.S. Pat. No. 6,447,766, the disclosure of which is incorporated herein by reference in its entirety.

The amino acid sequences of Gro-β, Gro-β T, Gro-β N69D, and Gro-β T N65D are set forth in TABLE 2, below.

TABLE 2
Amino acid sequences of Gro-β and
select variants thereof

| SEQ | | |
|---|---|---|
| ID | | |
| NO. | Description | Amino Acid Sequence |
| 1 | Gro-β | APLATELRCQCLQTLQGIHLKNIQ |
| | | SVKVKSPGPHCAQTEVIATLKNG |
| | | QKACLNPASPMVKKIIEKMLKNG |
| | | KSN |
| 2 | Gro-β-T | TELRCQCLQTLQGIHLKNIQSVKV |
| | | KSPGPHCAQTEVIATLKNGQKAC |
| | | LNPASPMVKKIIEKMLKNGKSN |
| 3 | Gro-β N69D | APLATELRCQCLQTLOGIHLKNIQ |
| | | SVKVKSPGPHCAQTEVIATLKNG |
| | | QKACLNPASPMVKKIIEKMLKDG |
| | | KSN |
| 4 | Gro-β-T N65D | TELRCQCLQTLQGIHLKNIQSVKV |
| | | KSPGPHCAQTEVIATLKNGQKAC |
| | | LNPASPMVKKIIEKMLKDGKSN |

Additional CXCR2 agonists that may be used in conjunction with the compositions and methods described herein include other variants of Gro-β, such as peptides that have one or more amino acid substitutions, insertions, and/or deletions relative to Gro-β. In some embodiments, CXCR2 agonists that may be used in conjunction with the compositions and methods described herein include peptides having at least 85% sequence identity to the amino acid sequence of SEQ ID NO: 1 (e.g., a peptide having at least 85%, 90%, 95%, 96%, 97%, 98%, 99%, 99.5%, or 100% sequence identity to the amino acid sequence of SEQ ID NO: 1). In some embodiments, the amino acid sequence of the CXCR2 agonist differs from that of SEQ ID NO: 1 only by way of one or more conservative amino acid substitutions. In some embodiments, in some embodiments, the amino acid sequence of the CXCR2 agonist differs from that of SEQ ID NO: 1 by no more than 20, no more than 15, no more than 10, no more than 5, or no more than 1 nonconservative amino acid substitutions. In some embodiments, the CXCR2 agonist is Gro-β. In some embodiments, the Gro-β T is not covalently modified. In some embodiments, the Gro-β is not covalently modified with a polyalkylene glycol moiety, such as a polyethylene glycol moiety.

Additional examples of CXCR2 agonists useful in conjunction with the compositions and methods described herein are variants of Gro-β T, such as peptides that have one or more amino acid substitutions, insertions, and/or deletions relative to Gro-β T. In some embodiments, the CXCR2 agonist may be a peptide having at least 85% sequence identity to the amino acid sequence of SEQ ID NO: 2 (e.g., a peptide having at least 85%, 90%, 95%, 96%, 97%, 98%, 99%, 99.5%, or 100% sequence identity to the amino acid sequence of SEQ ID NO: 2). In some embodiments, the amino acid sequence of the CXCR2 agonist differs from that of SEQ ID NO: 2 only by way of one or more conservative amino acid substitutions. In some embodiments, in some embodiments, the amino acid sequence of the CXCR2 agonist differs from that of SEQ ID NO: 2 by no more than 20, no more than 15, no more than 10, no more than 5, or no more than 1 nonconservative amino acid substitutions.

Additional examples of CXCR2 agonists useful in conjunction with the compositions and methods described herein are variants of Gro-β N69D, such as peptides that have one or more amino acid substitutions, insertions, and/or deletions relative to Gro-β N69D. In some embodiments, the CXCR2 agonist may be a peptide having at least 85% sequence identity to the amino acid sequence of SEQ ID NO: 3 (e.g., a peptide having at least 85%, 90%, 95%, 96%, 97%, 98%, 99%, 99.5%, or 100% sequence identity to the amino acid sequence of SEQ ID NO: 3). In some embodiments, the amino acid sequence of the CXCR2 agonist differs from that of SEQ ID NO: 3 only by way of one or more conservative amino acid substitutions. In some embodiments, in some embodiments, the amino acid sequence of the CXCR2 agonist differs from that of SEQ ID NO: 3 by no more than 20, no more than 15, no more than 10, no more than 5, or no more than 1 nonconservative amino acid substitutions.

Additional examples of CXCR2 agonists useful in conjunction with the compositions and methods described herein are variants of Gro-3 T N65D, such as peptides that have one or more amino acid substitutions, insertions, and/or deletions relative to Gro-β T N65D. In some embodiments, the CXCR2 agonist may be a peptide having at least 85% sequence identity to the amino acid sequence of SEQ ID NO: 4 (e.g., a peptide having at least 85%, 90%, 95%, 96%, 97%, 98%, 99%, 99.5%, or 100% sequence identity to the amino acid sequence of SEQ ID NO: 4). In some embodiments, the amino acid sequence of the CXCR2 agonist differs from that of SEQ ID NO: 4 only by way of one or more conservative amino acid substitutions. In some embodiments, in some embodiments, the amino acid sequence of the CXCR2 agonist differs from that of SEQ ID NO: 4 by no more than 20, no more than 15, no more than 10, no more than 5, or no more than 1 nonconservative amino acid substitutions.

### Agonistic Anti-CXCR2 Antibodies and Antigen-Binding Fragments Thereof

In some embodiments, the CXCR2 agonist is an antibody or antigen-binding fragment thereof that binds CXCR2 and activates CXCR2 signal transduction. In some embodiments, the CXCR2 agonist may be an antibody or antigen-binding fragment thereof that binds the same epitope on CXCR2 as Gro-β or a variant or truncation thereof, such as Gro-β T, as assessed, for example, by way of a competitive CXCR2 binding assay. In some embodiments, the CXCR2 agonist is an antibody or an antigen-binding fragment thereof that competes with Gro-β or a variant or truncation thereof, such as Gro-β T, for binding to CXCR2.

In some embodiments of any of the above aspects, the antibody or antigen-binding fragment thereof is selected from the group consisting of a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv. In some embodiments, the antibody has an isotype selected from the group consisting of IgG, IgA, IgM, IgD, and IgE.

### Synthetic CXCR2 Agonists

The peptidic CXCR2 agonists described herein, such as Gro-β, Gro-β T, and variants thereof, may be prepared synthetically, for instance, using solid phase peptide synthesis techniques. Systems and processes for performing solid phase peptide synthesis include those that are known in the art and have been described, for instance, in U.S. Pat. Nos. 9,169,287; 9,388,212; 9,206,222; 6,028,172; and 5,233,044, among others, the disclosures of each of which are incorporated herein by reference as they pertain to protocols and techniques for the synthesis of peptides on solid support. Solid phase peptide synthesis is a process in which amino acid residues are added to peptides that have been immobilized on a solid support, such as a polymeric resin (e.g., a hydrophilic resin, such as a polyethylene-glycol-containing resin, or hydrophobic resin, such as a polystyrene-based resin).

Peptides, such as those containing protecting groups at amino, hydroxy, thiol, and carboxy substituents, among others, may be bound to a solid support such that the peptide is effectively immobilized on the solid support. For example, the peptides may be bound to the solid support via their C termini, thereby immobilizing the peptides for subsequent reaction in at a resin-liquid interface.

The process of adding amino acid residues to immobilized peptides can include exposing a deprotection reagent to the immobilized peptides to remove at least a portion of the protection groups from at least a portion of the immobilized peptides. The deprotection reagent exposure step can be configured, for instance, such that side-chain protection groups are preserved, while N-terminal protection groups are removed. For instance, an exemplary amino protecting contains a fluorenylmethyloxycarbonyl (Fmoc) substituent. A deprotection reagent containing a strongly basic substance, such as piperidine (e.g., a piperidine solution in an appropriate organic solvent, such as dimethyl formamide (DMF)) may be exposed to the immobilized peptides such that the Fmoc protecting groups are removed from at least a portion of the immobilized peptides. Other protecting groups suitable for the protection of amino substituents include, for instance, the tert-butyloxycarbonyl (Boc) moiety. A deprotection reagent comprising a strong acid, such as trifluoroacetic acid (TFA) may be exposed to immobilized peptides containing a Boc-protected amino substituent so as to remove the Boc protecting group by an ionization process. In this way, peptides can be protected and deprotected at specific sites, such as at one or more side-chains or at the N- or C-terminus of an immobilized peptide so as to append chemical functionality regioselectively at one or more of these positions. This can be used, for instance, to derivatize a side-chain of an immobilized peptide, or to synthesize a peptide, e.g., from the C-terminus to the N-terminus.

The process of adding amino acid residues to immobilized peptides can include, for instance, exposing protected, activated amino acids to the immobilized peptides such that at least a portion of the activated amino acids are bonded to the immobilized peptides to form newly-bonded amino acid residues. For example, the peptides may be exposed to activated amino acids that react with the deprotected N-termini of the peptides so as to elongate the peptide chain by one amino acid. Amino acids can be activated for reaction with the deprotected peptides by reaction of the amino acid with an agent that enhances the electrophilicity of the backbone carbonyl carbon of the amino acid. For example, phosphonium and uronium salts can, in the presence of a tertiary base (e.g., diisopropylethylamine (DIPEA) and triethylamine (TEA), among others), convert protected amino acids into activated species (for example, BOP, PyBOP, HBTU, and TBTU all generate HOBt esters). Other reagents can be used to help prevent racemization that may be induced in the presence of a base. These reagents include carbodiimides (for example, DCC or WSCDI) with an added auxiliary nucleophile (for example, 1-hydroxy-benzotriazole (HOBt), 1-hydroxy-azabenzotriazole (HOAt), or HOSu) or derivatives thereof. Another reagent that can be utilized to prevent racemization is TBTU. The mixed anhydride method, using isobutyl chloroformate, with or without an added auxiliary nucleophile, can also be used, as well as the azide method, due to the low racemization associated with this reagent. These types of compounds can also increase the rate of carbodiimide-mediated couplings, as well as prevent dehydration of Asn and Gln residues. Typical additional reagents include also bases such as N,N-diisopropylethylamine (DIPEA), triethylamine (TEA) or N-methylmorpholine (NMM). These reagents are described in detail, for instance, in U.S. Pat. No. 8,546,350, the disclosure of which is incorporated herein in its entirety.

During the recombinant expression and folding of Gro-β and Gro-β T in aqueous solution, a particular C-terminal asparagine residue (Asn69 within Gro-β and Asn65 within Gro-β T) is prone to deamidation. This process effectuates the conversion of the asparagine residue to aspartic acid. Without wishing to be bound by any theory, the chemical synthesis of Gro-β and Gro-β T may overcome this problem, for instance, by providing conditions that reduce the exposure of this asparagine residue to nucleophilic solvent. When prepared synthetically (i.e., chemically synthesized), for instance, using, e.g., the solid phase peptide synthesis techniques described above, synthetic Gro-β, Gro-β T, and variants thereof that may be used in conjunction with the compositions and methods described herein may have a purity of, e.g., at least about 95% relative to the deamidated versions of these peptides (i.e., contain less than 5% of the corresponding deamidated peptide). For instance, synthetic Gro-R, Gro-β T, and variants thereof that may be used in conjunction with the compositions and methods described herein may have a purity of about 95%, 95.5%, 96%, 96.5%, 97%, 97.5%, 98%, 98.5%, 99%, 99.5%, 99.6%, 99.7%, 99.8%, 99.9%, 99.99%, or more, relative to the deamidated versions of these peptides (e.g., the Asn69 deamidated version of SEQ ID NO: 1 or the Asn65 deamidated version of SEQ ID NO: 2). For instance, synthetic Gro-β, Gro-β T, and variants thereof may have, for instance, a purity of from about 95% to about 99.99%, such as a purity of from about 95% to about 99.99%, about 96% to about 99.99%, about 97% to about 99.99%, about 98% to about 99.99%, about 99% to about 99.99%, about 99.9% to about 99.99%, about 95% to about 99.5%, about 96% to about 99.5%, about 95% to about 99%, or about 97% to about 99% relative to the deamidated versions of these peptides (e.g., the Asn69 deamidated version of SEQ ID NO: 1 or the Asn65 deamidated version of SEQ ID NO: 2).

### CXCR4 Antagonists

Exemplary CXCR4 antagonists for use in conjunction with the compositions and methods described herein are compounds represented by formula (I)

Z-linker-Z′ (I)

or a pharmaceutically acceptable salt thereof, wherein Z is:
(i) a cyclic polyamine containing from 9 to 32 ring members, wherein from 2 to 8 of the ring members are nitrogen atoms separated from one another by 2 or more carbon atoms; or
(ii) an amine represented by formula (IA)

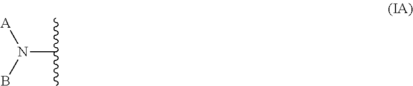

wherein A includes a monocyclic or bicyclic fused ring system including at least one nitrogen atom and B is H or a substituent of from 1 to 20 atoms;
and wherein Z′ is:
(i) a cyclic polyamine containing from 9 to 32 ring members, wherein from 2 to 8 of the ring members are nitrogen atoms separated from one another by 2 or more carbon atoms;
(ii) an amine represented by formula (IB)

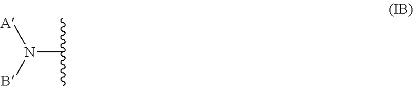

wherein A′ includes a monocyclic or bicyclic fused ring system including at least one nitrogen atom and B′ is H or a substituent of from 1 to 20 atoms; or
(iii) a substituent represented by formula (IC)

—N(R)—(CR₂)ₙ—X (IC)
wherein each R is independently H or C₁-C₆ alkyl, n is 1 or 2, and X is an aryl or heteroaryl group or a mercaptan;
   wherein the linker is a bond, optionally substituted alkylene (e.g., optionally substituted C₁-C₆ alkylene), optionally substituted heteroalkylene (e.g., optionally substituted C₁-C₆ heteroalkylene), optionally substituted alkenylene (e.g., optionally substituted C₂-C₆ alkenylene), optionally substituted heteroalkenylene (e.g., optionally substituted C₂-C₆ heteroalkenylene), optionally substituted alkynylene (e.g., optionally substituted C₂-C₆ alkynylene), optionally substituted heteroalkynylene (e.g., optionally substituted C₂-C₆ heteroalkynylene), optionally substituted cycloalkylene, optionally substituted heterocycloalkylene, optionally substituted arylene, or optionally substituted heteroarylene.

In some embodiments, Z and Z′ may each independently a cyclic polyamine containing from 9 to 32 ring members, of which from 2 to 8 are nitrogen atoms separated from one another by 2 or more carbon atoms. In some embodiments, Z and Z′ are identical substituents. As an example, Z may be a cyclic polyamine including from 10 to 24 ring members. In some embodiments, Z may be a cyclic polyamine that contains 14 ring members. In some embodiments, Z includes 4 nitrogen atoms. In some embodiments, Z is 1,4,8,11-tetraazocyclotetradecane.

In some embodiments, the linker is represented by formula (ID)

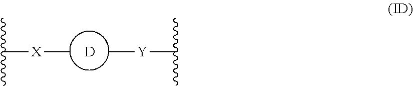

wherein ring D is an optionally substituted aryl group, an optionally substituted heteroaryl group, an optionally substituted cycloalkyl group, or an optionally substituted heterocycloalkyl group; and
X and Y are each independently optionally substituted alkylene (e.g., optionally substituted C₁-C₆ alkylene), optionally substituted heteroalkylene (e.g., optionally substituted C₁-C₆ heteroalkylene), optionally substituted alkenylene (e.g., optionally substituted C₂-C₆ alkenylene), optionally substituted heteroalkenylene (e.g., optionally substituted C₂-C₆ heteroalkenylene), optionally substituted alkynylene (e.g., optionally substituted C₂-C₆ alkynylene), or optionally substituted heteroalkynylene (e.g., optionally substituted C₂-C₆ heteroalkynylene).

As an example, the linker may be represented by formula (IE)

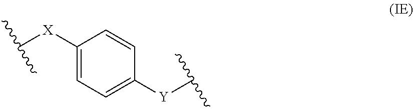

wherein ring D is an optionally substituted aryl group, an optionally substituted heteroaryl group, an optionally substituted cycloalkyl group, or an optionally substituted heterocycloalkyl group; and
X and Y are each independently optionally substituted alkylene (e.g., optionally substituted C₁-C₆ alkylene), optionally substituted heteroalkylene (e.g., optionally substituted C₁-C₆ heteroalkylene), optionally substituted C₂-C₆ alkenylene (e.g., optionally substituted C₂-C₆ alkenylene), optionally substituted heteroalkenylene (e.g., optionally substituted C₂-C₆ heteroalkenylene), optionally substituted alkynylene (e.g., optionally substituted C₂-C₆ alkynylene), or optionally substituted heteroalkynylene (e.g., optionally substituted C₂-C₆ heteroalkynylene). In some embodiments, X and Y are each independently optionally substituted C₁-C₆ alkylene. In some embodiments, X and Y are identical substituents. In some embodiments, X and Y may be each be methylene, ethylene, n-propylene, n-butylene, n-pentylene, or n-hexylene groups. In some embodiments, X and Y are each methylene groups.

The linker may be, for example, 1,3-phenylene, 2,6-pyridine, 3,5-pyridine, 2,5-thiophene, 4,4′-(2,2′-bipyrimidine), 2,9-(1,10-phenanthroline), or the like. In some embodiments, the linker is 1,4-phenylene-bis-(methylene).

CXCR4 antagonists useful in conjunction with the compositions and methods described herein include plerixafor (also referred to herein as “AMD3100” and “Mozibil”), or a pharmaceutically acceptable salt thereof, represented by formula (II), 1,1′-[1,4-phenylenebis(methylene)]-bis-1,4,8,11-tetra-azacyclotetradecane.

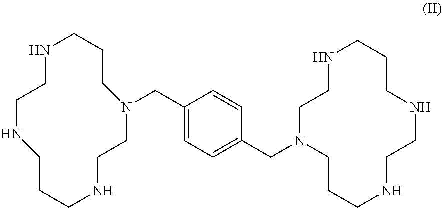 [SMILES: c1cc(CN2CCCNCCNCCCNCC2)ccc1CN1CCCNCCNCCCNCC1]

Additional CXCR4 antagonists that may be used in conjunction with the compositions and methods described herein include variants of plerixafor, such as a compound described in U.S. Pat. No. 5,583,131, the disclosure of which is incorporated herein by reference as it pertains to CXCR4 antagonists. In some embodiments, the CXCR4 antagonist may be a compound selected from the group consisting of: 1,1′-[1,3-phenylenebis(methylene)]-bis-1,4,8,11-tetra-azacyclotetradecane; 1,1′-[1,4-phenylene-bis-(methylene)]-bis-1,4,8,11-tetraazacyclotetradecane; bis-zinc or bis-copper complex of 1,1′-[1,4-phenylene-bis-(methylene)]-bis-1,4,8,11-tetraazacyclotetradecane; 1,1′-[3,3′-biphenylene-bis-(methylene)]-bis-1,4,8,11-tetraazacyclotetradecane; 11,11′-[1,4-phenylene-bis-(methylene)]-bis-1,4,7,11-tetraazacyclotetradecane; 1,11′-[1,4-phenylene-bis-(methylene)]-1,4,8,11-tetraazacyclotetradecane-1,4,7,11-tetraazacyclotetradecane; 1,1′-[2,6-pyridine-bis-(methylene)]-bis-1,4,8,11-tetraazacyclotetradecane; 1,1-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1′-[5-nitro-1,3-phenylenebis(methylene)]bis-1,4,8,11-tetraazacyclotetradecane; 1′,1′-[2,4,5,6-tetrachloro-1,3-phenyleneis(methylene)]bis-1,4,8,11-tetraazacyclotetradecane; 1,1′-[2,3,5,6-tetra-fluoro-1,4-phenylenebis(methylene)]bis-1,4,8,11-tetraazacyclotetradecane; 1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane; 1,1′-[1,4-phenylene-bis-(methylene)]-1,5,9-triazacyclododecane; 1,1′-[2,5-dimethyl-1,4-phenylenebis-(methylene)]-bis-1,4,8,11-tetraazacyclotetradecane; 1,1′-[2,5-dichloro-1,4-phenylenebis-(methylene)]-bis-1,4,8,11-tetraazacyclotetradecane; 1,1′-[2-bromo-1,4-phenylenebis-(methylene)]-bis-1,4,8,11-tetraazacyclotetradecane; and 1,1′-[6-phenyl-2,4-pyridinebis-(methylene)]-bis-1,4,8,11-tetraazacyclotetradecane.

In some embodiments, the CXCR4 antagonist is a compound described in U.S. 2006/0035829, the disclosure of which is incorporated herein by reference as it pertains to CXCR4 antagonists. In some embodiments, the CXCR4 antagonist may be a compound selected from the group consisting of 3,7,11,17-tetraazabicyclo(13.3.1)heptadeca-1(17),13,15-triene; 4,7,10,17-tetraazabicyclo(13.3.1)heptadeca-1(17),13,15-triene; 1,4,7,10-tetraazacyclotetradecane; 1,4,7-triazacyclotetradecane; and 4,7,10-triazabicyclo(13.3.1)heptadeca-1(17),13,15-triene.

The CXCR4 antagonist may be a compound described in WO 2001/044229, the disclosure of which is incorporated herein by reference as it pertains to CXCR4 antagonists. In some embodiments, the CXCR4 antagonist may be a compound selected from the group consisting of N-[4-(11-fluoro-1,4,7-triazacyclotetradecanyl)-1,4-phenylenebis(methylene)]-2-(aminomethyl)pyridine; N-[4-(11,11-difluoro-1,4,7-triazacyclotetradecanyl)-1,4-phenylenebis(methylene)]-2-(aminomethyl)pyridine; N-[4-(1,4,7-triazacyclotetradecan-2-onyl)-1,4-phenylenebis(methylene)]-2-(aminomethyl)pyridine; N-[12-(5-oxa-1,9-diazacyclotetradecanyl)-1,4-phenylenebis(methylene)]-2-(aminomethyl)pyridine; N-[4-(11-oxa-1,4,7-triazacyclotetradecanyl)-1,4-phenylenebis(methylene)]-2-(aminomethyl)pyridine; N-[4-(11-thia-1,4,7-triazacyclotetradecanyl)-1,4-phenylenebis(methylene)]-2-(aminomethyl)pyridine; N-[4-(11-sulfoxo-1,4,7-triazacyclotetradecanyl)-1,4-phenylenebis(methylene)]-2-(aminomethyl)pyridine; N-[4-(11-sulfono-1,4,7-triazacyclotetradecanyl)-1,4-phenylenebis(methylene)]-2-(aminomethyl)pyridine; and N-[4-(3-carboxo-1,4,7-triazacyclotetradecanyl)-1,4-phenylenebis(methylene)]-2-(aminomethyl)pyridine.

Additional CXCR4 antagonists useful in conjunction with the compositions and methods described herein include compounds described in WO 2000/002870, the disclosure of which is incorporated herein by reference as it pertains to CXCR4 antagonists. In some embodiments, the CXCR4 antagonist may be a compound selected from the group consisting of: N-[1,4,8,11-tetraazacyclotetra-decanyl-1,4-phenylenebis-(methylene)]-2-(aminomethyl)pyridine; N-[1,4,8,11-tetraazacyclotetra-decanyl-1,4-phenylenebis(methylene)]-N-methyl-2-(aminomethyl)pyridine; N-[1,4,8,11-tetraazacyclotetra-decanyl-1,4-phenylenebis(methylene)]-4-(aminomethyl)pyridine; N-[1,4,8,11-tetraazacyclotetra-decanyl-1,4-phenylenebis(methylene)]-3-(aminomethyl)pyridine; N-[1,4,8,11-tetraazacyclotetra-decanyl-1,4-phenylenebis(methylene)]-(2-aminomethyl-5-methyl)pyrazine; N-[1,4,8,11-tetraazacyclotetra-decanyl-1,4-phenylenebis(methylene)]-2-(aminoethyl) pyridine; N-[1,4,8,11-tetraazacyclotetra-decanyl-1,4-phenylenebis(methylene)]-2-(aminomethyl)thiophene; N-[1,4,8,11-tetraazacyclotetra-decanyl-1,4-phenylenebis(methylene)]-2-(aminomethyl)mercaptan; N-[1,4,8,11-tetraazacyclotetra-decanyl-1,4-phenylenebis(methylene)]-2-amino benzylamine; N-[1,4,8,11-tetraazacyclotetra-decanyl-1,4-phenylenebis(methylene)]-4-amino benzylamine; N-[1,4,8,11-tetraazacyclotetra-decanyl-1,4-phenylenebis(methylene)]-4-(aminoethyl)imidazole; N-[1,4,8,11-tetraazacyclotetra-decanyl-1,4-phenylenebis(methylene)]-benzylamine; N-[4-(1,4,7-triazacyclotetra-decanyl)-1,4-phenylenebis(methylene)]-2-(aminomethyl)pyridine; N-[7-(4,7,10,17-tetraazabicyclo[13.3.1]heptadeca-1(17),13,15-trienyl)-1,4-phenylenebis(methylene)]-2-(aminomethyl)pyridine; N-[7-(4,7,10-triazabicyclo[13.3.1]heptadeca-1(17),13,15-trienyl)-1,4-phenylenebis(methylene)]-2-(aminomethyl)pyridine; N-[1-(1,4,7-triazacyclotetra-decanyl)-1,4-phenylenebis(methylene)]-2-(aminomethyl)pyridine; N-[4-[4,7,10,17-tetraazabicyclo[13.3.1]heptadeca-1(17),13,15-trienyl]-1,4-phenylenebis(methylene)]-2-(aminomethyl)pyridine; N-[4-[4,7,10-triazabicyclo[13.3.1]heptadeca-1(17),13,15-trienyl]-1,4-phenylenebis(methylene)]-2-(aminomethyl)pyridine; N-[1,4,8,11-tetraazacyclotetradecanyl-1,4-phenylenebis(methylene)]-purine; 1-[1,4,8,11-tetraazacyclotetradecanyl-1,4-phenylenebix(methylene)]-4-phenylpiperazine; N-[4-(1,7-diazacyclotetradecanyl)-1,4-phenylenebis(methylene)]-2-(aminomethyl)pyridine; and N-[7-(4,10-diazabicyclo[13.3.1]heptadeca-1(17),13,15-trienyl)-1,4-phenylenebis(methylene)]-2-(aminomethyl)pyridine.

In some embodiments, the CXCR4 antagonist is a compound selected from the group consisting of: 1-[2,6-dimethoxypyrid-4-yl(methylene)]-1,4,8,11-tetraazacyclotetradecane; 1-[2-chloropyrid-4-yl(methylene)]-1,4,8,11-tetraazacyclotetradecane; 1-[2,6-dimethylpyrid-4-yl(methylene)]-1,4,8,11-tetraazacyclotetradecane; 1-[2-methylpyrid-4-yl(methylene)]-1,4,8,11-tetraazacyclotetradecane; 1-[2,6-dichloropyrid-4-yl(methylene)]-1,4,8,11-tetraazacyclotetradecane; 1-[2-chloropyrid-5-yl(methylene)]-1,4,8,11-tetraazacyclotetradecane; and 7-[4-methylphenyl (methylene)]-4,7,10,17-tetraazabicyclo[13.3.1]heptadeca-1(17),13,15-triene.

In some embodiments, the CXCR4 antagonist is a compound described in U.S. Pat. No. 5,698,546, the disclosure of which is incorporated herein by reference as it pertains to CXCR4 antagonists. In some embodiments, the CXCR4 antagonist may be a compound selected from the group consisting of: 7,7′-[1,4-phenylene-bis(methylene)]bis-3,7,11,17-tetraazabicyclo[13.3.1]heptadeca-1(17),13,15-triene; 7,7′-[1,4-phenylene-bis(methylene)]bis[15-chloro-3,7,11,17-tetraazabicyclo [13.3.1]heptadeca-1 (17),13,15-triene]; 7,7′-[1,4-phenylene-bis(methylene)]bis[15-methoxy-3,7,11,17-tetraazabicyclo[13.3.1]heptadeca-1(17),13,15-triene]; 7,7′-[1,4-phenylene-bis(methylene)]bis-3,7,11,17-tetraazabicyclo[13.3.1]-heptadeca-13,16-triene-15-one; 7,7′-[1,4-phenylene-bis(methylene)]bis-4,7,10,17-tetraazabicyclo[13.3.1]-heptadeca-1(17),13,15-triene; 8,8′-[1,4-phenylene-bis(methylene)]bis-4,8,12,19-tetraazabicyclo[15.3.1]nonadeca-1(19),15,17-triene; 6,6′-[1,4-phenylene-bis(methylene)]bis-3,6,9,15-tetraazabicyclo[11.3.1]pentadeca-1 (15),11,13-triene; 6,6′-[1,3-phenylene-bis(methylene)]bis-3,6,9,15-tetraazabicyclo[11.3.1]pentadeca-1 (15),11,13-triene; and 17,17′-[1,4-phenylene-bis(methylene)]bis-3,6,14,17,23,24-hexaazatricyclo[17.3.1.1^{8,12}]tetracosa-1(23),8,10,12(24),19,21-hexaene.

In some embodiments, the CXCR4 antagonist is a compound described in U.S. Pat. No. 5,021,409, the disclosure of which is incorporated herein by reference as it pertains to CXCR4 antagonists. In some embodiments, the CXCR4 antagonist may be a compound selected from the group consisting of: 2,2′-bicyclam, 6,6′-bicyclam; 3,3′-(bis-1,5,9,13-tetraaza cyclohexadecane); 3,3′-(bis-1,5,8,11,14-pentaazacyclohexadecane); methylene (or polymethylene) di-1-N-1,4,8,11-tetraaza cyclotetradecane; 3,3′-bis-1,5,9,13-tetraazacyclohexadecane; 3,3′-bis-1,5,8,11,14-pentaazacyclohexadecane; 5,5′-bis-1,4,8,11-tetraazacyclotetradecane; 2,5′-bis-1,4,8,11-tetraazacyclotetradecane; 2,6′-bis-1,4,8,11-tetraazacyclotetradecane; 11,11′-(1,2-ethanediyl)bis-1,4,8,11-tetraazacyclotetradecane; 11,11′-(1,2-propanediyl)bis-1,4,8,11-tetraazacyclotetradecane; 11,11′-(1,2-butanediyl)bis-1,4,8,11-tetraazacyclotetradecane; 11,11′-(1,2-pentanediyl)bis-1,4,8,11-tetraazacyclotetradecane; and 11,11′-(1,2-hexanediyl)bis-1,4,8,11-tetraazacyclotetradecane.

In some embodiments, the CXCR4 antagonist is a compound described in WO 2000/056729, the disclosure of which is incorporated herein by reference as it pertains to CXCR4 antagonists. In some embodiments, the CXCR4 antagonist may be a compound selected from the group consisting of N-(2-pyridinylmethyl)-N′-(6,7,8,9-tetrahydro-5H-cyclohepta[b]pyridin-9-yl)-1,4-benzenedimethanamine; N-(2-pyridinylmethyl)-N′-(5,6,7,8-tetrahydro-8-quinolinyl)-1,4-benzenedimethanamine; N-(2-pyridinylmethyl)-N′-(6,7-dihydro-5H-cyclopenta[b]pyridin-7-yl)-1,4-benzenedimethanamine; N-(2-pyridinylmethyl)-N′-(1,2,3,4-tetrahydro-1-naphthalenyl)-1,4-benzenedimethanamine; N-(2-pyridinylmethyl)-N′-(1-naphthalenyl)-1,4-benzenedimethanamine; N-(2-pyridinylmethyl)-N′-(8-quinolinyl)-1,4-benzenedimethanamine; N-(2-pyridinylmethyl)-N′-[2-[(2-pyridinylmethyl)amino]ethyl]-N′-(1-methyl-1,2,3,4-tetrahydro-8-quinolinyl)-1,4-benzenedimethanamine; N-(2-pyridinylmethyl)-N′-[2-[(1H-imidazol-2-ylmethyl)amino]ethyl]-N′-(1-methyl-1,2,3,4-tetrahydro-8-quinolinyl)-1,4-benzenedimethanamine; N-(2-pyridinylmethyl)-N′-(1,2,3,4-tetrahydro-8-quinolinyl)-1,4-benzenedimethanamine; N-(2-pyridinylmethyl)-N′-[2-[(1H-imidazol-2-ylmethyl)amino]ethyl]-N′-(1,2,3,4-tetrahydro-1-naphthalenyl)-1,4-benzenedimethanamine; N-(2-pyridinylmethyl)-N′-(2-phenyl-5,6,7,8-tetrahydro-8-quinolinyl)-1,4-benzenedimethanamine; N,N′-bis(2-pyridinylmethyl)-N′-(2-phenyl-5,6,7,8-tetrahydro-8-quinolinyl)-1,4-benzenedimethanamine;

N-(2-pyridinylmethyl)-N′-(5,6,7,8-tetrahydro-5-quinolinyl)-1,4-benzenedimethanamine; N-(2-pyridinylmethyl)-N′-(1H-imidazol-2-ylmethyl)-N′-(5,6,7,8-tetrahydro-5-quinolinyl)-1,4-benzenedimethanamine; N-(2-pyridinylmethyl)-N′-(1H-imidazol-2-ylmethyl)-N′-(5,6,7,8-tetrahydro-8-quinolinyl)-1,4-benzenedimethanamine; N-(2-pyridinylmethyl)-N′-[(2-amino-3-phenyl)propyl]-N′-(5,6,7,8-tetrahydro-8-quinolinyl)-1,4-benzenedimethanamine; N-(2-pyridinylmethyl)-N′-(1H-imidazol-4-ylmethyl)-N′-(5,6,7,8-tetrahydro-8-quinolinyl)-1,4-benzenedimethanamine; N-(2-pyridinylmethyl)-N′-(2-quinolinylmethyl)-N′-(5,6,7,8-tetrahydro-8-quinolinyl)-1,4-benzenedimethanamine; N-(2-pyridinylmethyl)-N′-(2-(2-naphthoyl)aminoethyl)-N′-(5,6,7,8-tetrahydro-8-quinolinyl)-1,4-benzenedimethanamine; N-(2-pyridinylmethyl)-N′—[(S)-(2-acetylamino-3-phenyl)propyl]-N′-(5,6,7,8-tetrahydro-8-quinolinyl)-1,4-benzenedimethanamine; N-(2-pyridinylmethyl)-N′—[(S)-(2-acetylamino-3-phenyl)propyl]-N′-(5,6,7,8-tetrahydro-8-quinolinyl)-1,4-benzenedimethanamine; N-(2-pyridinylmethyl)-N′-[3-((2-naphthalenylmethyl)amino)propyl]-N′-(5,6,7,8-tetrahydro-8-quinolinyl)-1,4-benzenedimethanamine; N-(2-pyridinylmethyl)-N′-[2-(S)-pyrollidinylmethyl]-N′-(5,6,7,8-tetrahydro-8-quinolinyl)-1,4-benzenedimethanamine; N-(2-pyridinylmethyl)-N′-[2-(R)-pyrollidinylmethyl]-N′-(5,6,7,8-tetrahydro-8-quinolinyl)-1,4-benzenedimethanamine; N-(2-pyridinylmethyl)-N′-[3-pyrazolylmethyl]-N′-(5,6,7,8-tetrahydro-8-quinolinyl)-1,4-benzenedimethanamine; N-(2-pyridinylmethyl)-N′-[2-pyrrolylmethyl]-N′-(5,6,7,8-tetrahydro-8-quinolinyl)-1,4-benzenedimethanamine; N-(2-pyridinylmethyl)-N′-[2-thiopheneylmethyl]-N′-(5,6,7,8-tetrahydro-8-quinolinyl)-1,4-benzenedimethanamine; N-(2-pyridinylmethyl)-N′-[2-thiazolylmethyl]-N′-(5,6,7,8-tetrahydro-8-quinolinyl)-1,4-benzenedimethanamine; N-(2-pyridinylmethyl)-N′-[2-furanylmethyl]-N′-(5,6,7,8-tetrahydro-8-quinolinyl)-1,4-benzenedimethanamine; N-(2-pyridinylmethyl)-N′-[2-[(phenylmethyl)amino]ethyl]-N′-(5,6,7,8-tetrahydro-8-quinolinyl)-1,4-benzenedimethanamine; N-(2-pyridinylmethyl)-N′-(2-aminoethyl)-N′-(5,6,7,8-tetrahydro-8-quinolinyl)-1,4-benzenedimethanamine; N-(2-pyridinylmethyl)-N′-3-pyrrolidinyl-N′-(5,6,7,8-tetrahydro-8-quinolinyl)-1,4-benzenedimethanamine; N-(2-pyridinylmethyl)-N′-4-piperidinyl-N′-(5,6,7,8-tetrahydro-8-quinolinyl)-1,4-benzenedimethanamine; N-(2-pyridinylmethyl)-N′-[2-[(phenyl)amino]ethyl]-N′-(5,6,7,8-tetrahydro-8-quinolinyl)-1,4-benzenedimethanamine; N-(2-pyridinylmethyl)-N′-(7-methoxy-1,2,3,4-tetrahydro-2-naphthalenyl)-1,4-benzenedimethanamine; N-(2-pyridinylmethyl)-N′-(6-methoxy-1,2,3,4-tetrahydro-2-naphthalenyl)-1,4-benzenedimethanamine; N-(2-pyridinylmethyl)-N′-(1-methyl-1,2,3,4-tetrahydro-2-naphthalenyl)-1,4-benzenedimethanamine; N-(2-pyridinylmethyl)-N′-(7-methoxy-3,4-dihydronaphthalenyl)-1-(aminomethyl)-4-benzamide; N-(2-pyridinylmethyl)-N′-(6-methoxy-3,4-dihydronaphthalenyl)-1-(aminomethyl)-4-benzamide; N-(2-pyridinylmethyl)-N′-(1H-imidazol-2-ylmethyl)-N′-(7-methoxy-1,2,3,4-tetrahydro-2-naphthalenyl)-1,4-benzenedimethanamine; N-(2-pyridinylmethyl)-N′-(8-hydroxy-1,2,3,4-tetrahydro-2-naphthalenyl)-1,4-benzenedimethanamine; N-(2-pyridinylmethyl)-N′-(1H-imidazol-2-ylmethyl)-N′-(8-hydroxy-1,2,3,4-tetrahydro-2-naphthalenyl)-1,4-benzenedimethanamine; N-(2-pyridinylmethyl)-N′-(8-Fluoro-1,2,3,4-tetrahydro-2-naphthalenyl)-1,4-benzenedimethanamine; N-(2-pyridinylmethyl)-N′-(1H-imidazol-2-ylmethyl)-N′-(8-Fluoro-1,2,3,4-tetrahydro-2-naphthalenyl)-1,4-benzenedimethanamine; N-(2-pyridinylmethyl)-N′-(5,6,7,8-tetrahydro-7-quinolinyl)-1,4-benzenedimethanamine; N-(2-pyridinylmethyl)-N′-(1H-imidazol-2-ylmethyl)-N′-(5,6,7,8-tetrahydro-7-quinolinyl)-1,4-benzenedimethanamine; N-(2-pyridinylmethyl)-N′-[2-[(2-naphthalenylmethyl)amino]ethyl]-N′-(5,6,7,8-tetrahydro-8-quinolinyl)-1,4-benzenedimethanamine; N-(2-pyridinylmethyl)-N′-[2-(isobutylamino)ethyl]-N′-(5,6,7,8-tetrahydro-8-quinolinyl)-1,4-benzenedimethanamine; N-(2-pyridinylmethyl)-N′-[2-[(2-pyridinylmethyl)amino]ethyl]-N′-(5,6,7,8-tetrahydro-8-quinolinyl)-1,4-benzenedimethanamine; N-(2-pyridinylmethyl)-N′-[2-[(2-furanylmethyl)amino]ethyl]-N′-(5,6,7,8-tetrahydro-8-quinolinyl)-1,4-benzenedimethanamine; N-(2-pyridinylmethyl)-N′-(2-guanidinoethyl)-N′-(5,6,7,8-tetrahydro-8-quinolinyl)-1,4-benzenedimethanamine; N-(2-pyridinylmethyl)-N′-[2-[bis-[(2-methoxy)phenylmethyl]amino]ethyl]-N′-(5,6,7,8-tetrahydro-8-quinolinyl)-1,4-benzenedimethanamine; N-(2-pyridinylmethyl)-N′-[2-[(1H-imidazol-4-ylmethyl)amino]ethyl]-N′-(5,6,7,8-tetrahydro-8-quinolinyl)-1,4-benzenedimethanamine; N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine; N-(2-pyridinylmethyl)-N′-[2-(phenylureido)ethyl]-N′-(5,6,7,8-tetrahydro-8-quinolinyl)-1,4-benzenedimethanamine; N-(2-pyridinylmethyl)-N′—[[N″-(n-butyl)carboxamido]methyl]-N′-(5,6,7,8-tetrahydro-8-quinolinyl)-1,4-benzenedimethanamine; N-(2-pyridinylmethyl)-N′-(carboxamidomethyl)-N′-(5,6,7,8-tetrahydro-8-quinolinyl)-1,4-benzenedimethanamine; N-(2-pyridinylmethyl)-N′—[(N″-phenyl)carboxamidomethyl]-N′-(5,6,7,8-tetrahydro-8-quinolinyl)-1,4-benzenedimethanamine; N-(2-pyridinylmethyl)-N′-(carboxymethyl)-N′-(5,6,7,8-tetrahydro-8-quinolinyl)-1,4-benzenedimethanamine; N-(2-pyridinylmethyl)-N′-(phenylmethyl)-N′-(5,6,7,8-tetrahydro-8-quinolinyl)-1,4-benzenedimethanamine; N-(2-pyridinylmethyl)-N′-(1H-benzimidazol-2-ylmethyl)-N′-(5,6,7,8-tetrahydro-8-quinolinyl)-1,4-benzenedimethanamine; N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine (hydrobromide salt); N-(2-pyridinylmethyl)-N′-(5-nitro-1H-benzimidazol-2-ylmethyl)-N′-(5,6,7,8-tetrahydro-8-quinolinyl)-1,4-benzenedimethanamine; N-(2-pyridinylmethyl)-N′-[(1H)-5-azabenzimidazol-2-ylmethyl]-N′-(5,6,7,8-tetrahydro-8-quinolinyl)-1,4-benzenedimethanamine; N-(2-pyridinylmethyl)-N-(4-phenyl-1H-imidazol-2-ylmethyl)-N′-(5,6,7,8-tetrahydro-8-quinolinyl)-1,4-benzenedimethanamine; N-(2-pyridinylmethyl)-N′-[2-(2-pyridinyl)ethyl]-N′-(5,6,7,8-tetrahydro-8-quinolinyl)-1,4-benzenedimethanamine; N-(2-pyridinylmethyl)-N′-(2-benzoxazolyl)-N′-(5,6,7,8-tetrahydro-8-quinolinyl)-1,4-benzenedimethanamine; N-(2-pyridinylmethyl)-N′-(trans-2-aminocyclohexyl)-N′-(5,6,7,8-tetrahydro-8-quinolinyl)-1,4-benzenedimethanamine; N-(2-pyridinylmethyl)-N′-(2-phenylethyl)-N′-(5,6,7,8-tetrahydro-8-quinolinyl)-1,4-benzenedimethanamine; N-(2-pyridinylmethyl)-N′-(3-phenylpropyl)-N′-(5,6,7,8-tetrahydro-8-quinolinyl)-1,4-benzenedimethanamine; N-(2-pyridinylmethyl)-N′-(trans-2-aminocyclopentyl)-N′-(5,6,7,8-tetrahydro-8-quinolinyl)-1,4-benzenedimethanamine; N-[[4-[[(2-pyridinylmethyl)amino]methyl]phenyl]methyl]-N-(5,6,7,8-tetrahydro-8-quinolinyl)-glycinamide; N-[[4-[[(2-pyridinylmethyl)amino]methyl]phenyl]methyl]-N-(5,6,7,8-tetrahydro-8-quinolinyl)-(L)-alaninamide; N-[[4-[[(2-pyridinylmethyl)amino]methyl]phenyl]methyl]-N-(5,6,7,8-tetrahydro-8-quinolinyl)-(L)-aspartamide; N-[[4-[[(2-pyridinylmethyl)amino]methyl]phenyl]methyl]-N-(5,6,7,8-tetrahydro-8-quinolinyl)-pyrazinamide; N-[[4-[[(2-pyridinylmethyl)amino]methyl]phenyl]methyl]-N-(5,6,7,8-tetrahydro-8-quinolinyl)-(L)-prolinamide; N-[[4-[[(2-pyridinylmethyl)amino]methyl]phenyl]methyl]-N-(5,6,7,8-tetrahydro-8-quinolinyl)-(L)-lysinamide; N-[[4-[[(2-pyridinylmethyl)amino]methyl]phenyl]methyl]-N-(5,6,7,8-tetrahydro-8-quinolinyl)-benzamide; N-[[4-[[(2-pyridinylmethyl)amino]methyl]phenyl]methyl]-N-(5,6,7,8-tetrahydro-8-quinolinyl)-picolinamide; N′-Benzyl-N-[[4-[[(2-pyridinylmethyl)amino]methyl]phenyl]methyl]-N-(5,6,7,8-tetrahydro-8-quinolinyl)-urea; N′-phenyl-N-[[4-[[(2-pyridinylmethyl)amino]methyl]phenyl]methyl]-N-(5,6,7,8-tetrahydro-8-quinolinyl)-urea; N-(6,7,8,9-tetrahydro-5H-cyclohepta[bacteriapyridin-9-yl)-4-[[(2-pyridinylmethyl)amino]methyl]benzamide; N-(5,6,7,8-tetrahydro-8-quinolinyl)-4-[[(2-pyridinylmethyl)amino]methyl]benzamide; N,N′-bis(2-pyridinylmethyl)-N′-(5,6,7,8-tetrahydro-8-quinolinyl)-1,4-benzenedimethanamine; N,N′-bis(2-pyridinylmethyl)-N′-(6,7,8,9-tetrahydro-5H-cyclohepta[bacteriapyridin-9-yl)-1,4-benzenedimethanamine; N,N′-bis(2-pyridinylmethyl)-N′-(6,7-dihydro-5H-cyclopenta[bacteriapyridin-7-yl)-1,4-benzenedimethanamine; N,N′-bis(2-pyridinylmethyl)-N′-(1,2,3,4-tetrahydro-1-naphthalenyl)-1,4-benzenedimethanamine; N,N′-bis(2-pyridinylmethyl)-N′-[(5,6,7,8-tetrahydro-8-quinolinyl)methyl]-1,4-benzenedimethanamine; N,N′-bis(2-pyridinylmethyl)-N′[(6,7-dihydro-5H-cyclopenta[bacteriapyridin-7-yl)methyl]-1,4-benzenedimethanamine; N-(2-pyridinylmethyl)-N-(2-methoxyethyl)-N′-(5,6,7,8-tetrahydro-8-quinolinyl)-1,4-benzenedimethanamine; N-(2-pyridinylmethyl)-N-[2-(4-methoxyphenyl)ethyl]-N′-(5,6,7,8-tetrahydro-8-quinolinyl)-1,4-benzenedimethanamine; N,N′-bis(2-pyridinylmethyl)-1,4-(5,6,7,8-tetrahydro-8-quinolinyl)benzenedimethanamine; N-[(2,3-dimethoxyphenyl)methyl]-N′-(2-pyridinylmethyl)-N-(5,6,7,8-tetrahydro-8-quinolinyl)-1,4-benzenedimethanamine; N,N′-bis(2-pyridinylmethyl)-N-[1-(N″-phenyl-N″-methylureido)-4-piperidinyl]-1,3-benzenedimethanamine; N,N′-bis(2-pyridinylmethyl)-N—[N″-p-toluenesulfonylphenylalanyl)-4-piperidinyl]-1,3-benzenedimethanamine; N,N′-bis(2-pyridinylmethyl)-N-[1-[3-(2-chlorophenyl)-5-methyl-isoxazol-4-oyl]-4-piperidinyl]-1,3-benzenedimethanamine; N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine; N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine; N-[(4-cyanophenyl)methyl]-N′-(2-pyridinylmethyl)-N-(5,6,7,8-tetrahydro-8-quinolinyl)-1,4-benzenedimethanamine; N-[(4-acetamidophenyl)methyl]-N′-(2-pyridinylmethyl)-N-(5,6,7,8-tetrahydro-8-quinolinyl)-1,4-benzenedimethanamine; N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine; N-[(1-methyl-2-carboxamido)ethyl]-N,N′-bis(2-pyridinylmethyl)-1,3-benzenedimethanamine; N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine; N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine; N-[1-(benzyl)-3-pyrrolidinyl]-N,N′-bis(2-pyridinylmethyl)-1,3-benzenedimethanamine; N-[[1-methyl-3-(pyrazol-3-yl)]propyl]-N,N′-bis(2-pyridinylmethyl)-1,3-benzenedimethanamine; N-[1-(phenyl)ethyl]-N,N′-bis(2-pyridinylmethyl)-1,3-benzenedimethanamine; N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine; N-[1-benzyl-3-carboxymethyl-4-piperidinyl]-N,N′-bis(2-pyridinylmethyl)-1,3-benzenedimethanamine; N-[(3,4-methylenedioxyphenyl)methyl]-N′-(2-pyridinylmethyl)-N-(5,6,7,8-tetrahydro-8-quinolinyl)-1,4-benzenedimethanamine; N-(3-pyridinylmethyl)-N′-(2-pyridinylmethyl)-N-(6,7,8,9-tetrahydro-5H-cyclohepta[b]pyridin-9-yl)-1,4-benzenedimethanamine; N-[[1-methyl-2-(2-tolyl)carboxamido]ethyl]-N,N′-bis(2-pyridinylmethyl)-1,3-benzenedimethanamine; N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine; N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine; N-(1-phenyl-3,5-dimethylpyrazolin− 4-ylmethyl)-N′-(2-pyridinylmethyl)-N-(5,6,7,8-tetrahydro-8-quinolinyl)-1,4-benzenedimethanamine; N—[H-imidazol-4-ylmethyl]-N,N′-bis(2-pyridinylmethyl)-1,3-benzenedimethanamine; N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine; N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine; N-[(3-cyanophenyl)methyl]-N′-(2-pyridinylmethyl)-N-(5,6,7,8-tetrahydro-8-quinolinyl)-1,4-benzenedimethanamine; N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine; N-(5-ethylthiophene-2-ylmethyl)-N′-(2-pyridinylmethyl)-N-(5,6,7,8-tetrahydro-8-quinolinyl)-1,4-benzenedimethanamine; N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine; N-[(2,6-difluorophenyl)methyl]-N′-(2-pyridinylmethyl)-N-(5,6,7,8-tetrahydro-8-quinolinyl)-1,4-benzenedimethanamine; N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine; N-(2-difluoromethoxyphenylmethyl)-N′-(2-pyridinylmethyl)-N-(5,6,7,8-tetrahydro-8-quinolinyl)-1,4-benzenedimethanamine; N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine; N,N′-bis(2-pyridinylmethyl)-N-[1-(N″-phenyl-N″-methylureido)-4-piperidinyl]-1,4-benzenedimethanamine; N,N′-bis(2-pyridinylmethyl)-N—[N″-p-toluenesulfonylphenylalanyl)-4-piperidinyl]-1,4-benzenedimethanamine; N-[1-(3-pyridinecarboxamido)-4-piperidinyl]-N,N′-bis(2-pyridinylmethyl)-1,4-benzenedimethanamine; N-[1-(cyclopropylcarboxamido)-4-piperidinyl]-N,N′-bis(2-pyridinylmethyl)-1,4-benzenedimethanamine; N-[1-(1-phenylcyclopropylcarboxamido)-4-piperidinyl]-N,N′-bis(2-pyridinylmethyl)-1,4-benzenedimethanamine; N-(1,4-benzodioxan-6-ylmethyl)-N′-(2-pyridinylmethyl)-N-(5,6,7,8-tetrahydro-8-quinolinyl)-1,4-benzenedimethanamine; N-[1-[3-(2-chlorophenyl)-5-methyl-isoxazol-4-carboxamido]-4-piperidinyl]-N,N′-bis(2-pyridinylmethyl)-1,4-benzenedimethanamine; N-[1-(2-thiomethylpyridine-3-carboxamido)-4-piperidinyl]-N,N′-bis(2-pyridinylmethyl)-1,4-benzenedimethanamine; N-[(2,4-difluorophenyl)methyl]-N′-(2-pyridinylmethyl)-N-(5,6,7,8-tetrahydro-8-quinolinyl)-1,4-benzenedimethanamine; N-(1-methylpyrrol-2-ylmethyl)-N′-(2-pyridinylmethyl)-N-(5,6,7,8-tetrahydro-8-quinolinyl)-1,4-benzenedimethanamine; N-[(2-hydroxyphenyl)methyl]-N′-(2-pyridinylmethyl)-N-(5,6,7,8-tetrahydro-8-quinolinyl)-1,4-benzenedimethanamine; N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine; N-(3-pyridinylmethyl)-N′-(2-pyridinylmethyl)-N-(5,6,7,8-tetrahydro-8-quinolinyl)-1,4-benzenedimethanamine; N-[2-(N″-morpholinomethyl)-1-cyclopentyl]-N,N′-bis(2-pyridinylmethyl)-1,4-benzenedimethanamine; N-[(1-methyl-3-piperidinyl)propyl]-N,N′-bis(2-pyridinylmethyl)-1,4-benzenedimethanamine; N-(1-methylbenzimidazol-2-ylmethyl)-N′-(2-pyridinylmethyl)-N-(5,6,7,8-tetrahydro-8-quinolinyl)-1,4-benzenedimethanamine; N-[1-(benzyl)-3-pyrrol idinyl]-N,N′-bis(2-pyridinylmethyl)-1,4-benzenedimethanamine; N-[[(1-phenyl-3-(N″-morpholino)]propyl]-N,N′-bis(2-pyridinylmethyl)-1,4-benzenedimethanamine; N-[1-(iso-propyl)-4-piperidinyl]-N,N′-bis(2-pyridinylmethyl)-1,4-benzenedimethanamine; N-[1-(ethoxycarbonyl)-4-piperidinyl]-N′-(2-pyridinylmethyl)-N-(5,6,7,8-tetrahydro-8-quinolinyl)-1,4-benzenedimethanamine; N-[(1-methyl-3-pyrazolyl)propyl]-N′-(2-pyridinylmethyl)-N-(5,6,7,8-tetrahydro-8-quinolinyl)-1,4-benzenedimethanamine; N-[1-methyl-2-(N″,N″-diethylcarboxamido)ethyl]-N,N′-bis(2-pyridinylmethyl)-1,4-benzenedimethanamine; N-[(1-methyl-2-phenylsulfonyl)ethyl]-N′-(2-pyridinylmethyl)-N-(5,6,7,8-tetrahydro-8-quinolinyl)-1,4-benzenedimethanamine; N-[(2-chloro-4,5-methylenedioxyphenyl)methyl]-N′-(2-pyridinylmethyl)-N-(5,6,7,8-tetrahydro-8-quinolinyl)-1,4-benzenedimethanamine; N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine; N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine; N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine; N-(3-quinolylmethyl)-N′-(2-pyridinylmethyl)-N-(5,6,7,8-tetrahydro-8-quinolinyl)-1,4-benzenedimethanamine; N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine; N-(2-quinolylmethyl)-N′-(2-pyridinylmethyl)-N-(6,7,8,9-tetrahydro-5H-cyclohepta[b]pyridin-9-yl)-1,4-benzenedimethanamine; N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine; N-[1H-imidazol-2-ylmethyl]-N,N′-bis(2-pyridinylmethyl)-1,4-benzenedimethanamine; N-(3-quinolylmethyl)-N′-(2-pyridinylmethyl)-N-(6,7,8,9-tetrahydro-5H-cyclohepta[b]pyridin-9-yl)-1,4-benzenedimethanamine; N-(2-thiazolylmethyl)-N′-(2-pyridinylmethyl)-N-(6,7,8,9-tetrahydro-5H-cyclohepta[b]pyridin-9-yl)-1,4-benzenedimethanamine; N-(4-pyridinylmethyl)-N′-(2-pyridinylmethyl)-N-(6,7,8,9-tetrahydro-5H-cyclohepta[b]pyridin-9-yl)-1,4-benzenedimethanamine; N-[(5-benzyloxy)benzo[b]pyrrol-3-ylmethyl]-N,N′-bis(2-pyridinylmethyl)-1,4-benzenedimethanamine; N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine; N-[(4-methyl)-1H-imidazol-5-ylmethyl]-N,N′-bis(2-pyridinylmethyl)-1,4-benzenedimethanamine; N-[[(4-dimethylamino)-1-napthalenyl]methyl]-N,N′-bis(2-pyridinylmethyl)-1,4-benzenedimethanamine; N-[1,5-dimethyl-2-phenyl-3-pyrazolinone-4-ylmethyl]-N,N′-bis(2-pyridinylmethyl)-1,4-benzenedimethanamine; N-[1-[(1-acetyl-2-(R)-prolinyl]-4-piperidinyl]-N-[2-(2-pyridinyl)ethyl]-N′-(2-pyridinylmethyl)-1,3-benzenedimethanamine; N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine; N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine; N—[(N″-acetyltryptophanyl)-4-piperidinyl]-N-[2-(2-pyridinyl)ethyl]-N′-(2-pyridinylmethyl)-1,3-benzenedimethanamine; N—[(N″-benzoylvalinyl)-4-piperidinyl]-N-[2-(2-pyridinyl)ethyl]-N′-(2-pyridinylmethyl)-1,3-benzenedimethanamine; N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine; N-(4-pyridinylmethyl)-N′-(2-pyridinylmethyl)-N-(5,6,7,8-tetrahydro-8-quinolinyl)-1,4-benzenedimethanamine; N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine; N-[1-butyl-4-piperidinyl]-N-[2-(2-pyridinyl)ethyl]-N′-(2-pyridinylmethyl)-1,3-benzenedimethanamine; N-[1-benzoyl-4-piperidinyl]-N-[2-(2-pyridinyl)ethyl]-N′-(2-pyridinylmethyl)-1,3-benzenedimethanamine; N-[1-(benzyl)-3-pyrrolidinyl]-N-[2-(2-pyridinyl)ethyl]-N′-(2-pyridinylmethyl)-1,3-benzenedimethanamine; N-[(1-methyl)benzo[b]pyrrol-3-ylmethyl]-N-[2-(2-pyridinyl)ethyl]-N′-(2-pyridinylmethyl)-1,3-benzenedimethanamine; N-[1H-imidazol-4-ylmethyl]-N-[2-(2-pyridinyl)ethyl]-N′-(2-pyridinylmethyl)-1,3-benzenedimethanamine; N-[1-(benzyl)-4-piperidinyl]-N-[2-(2-pyridinyl)ethyl]-N′-(2-pyridinylmethyl)-1,4-benzenedimethanamine; N-[1-methylbenzimidazol-2-ylmethyl]-N-[2-(2-pyridinyl)ethyl]-N′-(2-pyridinylmethyl)-1,4-benzenedimethanamine; N-[(2-phenyl)benzo[b]pyrrol-3-ylmethyl]-N-[2-(2-pyridinyl)ethyl]-N′-(2-pyridinylmethyl)-1,4-benzenedimethanamine; N-[(6-methylpyridin-2-yl)methyl]-N′-(2-pyridinylmethyl)-N-(5,6,7,8-tetrahydro-8-quinolinyl)-1,4-benzenedimethanamine; N-(3-methyl-1H-pyrazol-5-ylmethyl)-N′-(2-pyridinylmethyl)-N-(5,6,7,8-tetrahydro-8-quinolinyl)-1,3-benzenedimethanamine; N-[(2-methoxyphenyl)methyl]-N′-(2-pyridinylmethyl)-N-(5,6,7,8-tetrahydro-8-quinolinyl)-1,3-benzenedimethanamine; N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine; N-(benzyloxyethyl)-N′-(2-pyridinylmethyl)-N-(5,6,7,8-tetrahydro-8-quinolinyl)-1,3-benzenedimethanamine; N-[(2-ethoxy-1-naphthalenyl)methyl]-N′-(2-pyridinylmethyl)-N-(5,6,7,8-tetrahydro-8-quinolinyl)-1,3-benzenedimethanamine; N-[(6-methylpyridin-2-yl)methyl]-N′-(2-pyridinylmethyl)-N-(5,6,7,8-tetrahydro-8-quinolinyl)-1,3-benzenedimethanamine; 1-[[4-[[(2-pyridinylmethyl)amino]methyl]phenyl]methyl]guanidine; N-(2-pyridinylmethyl)-N-(8-methyl-8-azabicyclo[3.2.1]octan-3-yl)-1,4-benzenedimethanamine; 1-[[4-[[(2-pyridinylmethyl)amino]methyl]phenyl]methyl]homopiperazine; 1-[[3-[[(2-pyridinylmethyl)amino]methyl]phenyl]methyl]homopiperazine; trans and cis-1-[[4-[[(2-pyridinylmethyl)amino]methyl]phenyl]methyl]-3,5-piperidinediamine; N,N′-[1,4-Phenylenebis(methylene)]bis-4-(2-pyrimidyl)piperazine; 1-[[4-[[(2-pyridinylmethyl)amino]methyl]phenyl]methyl]-1-(2-pyridinyl)methylamine; 2-(2-pyridinyl)-5-[[(2-pyridinylmethyl)amino]methyl]-1,2,3,4-tetrahydroisoquinoline; 1-[[4-[[(2-pyridinylmethyl)amino]methyl]phenyl]methyl]-3,4-diaminopyrrolidine; 1-[[4-[[(2-pyridinylmethyl)amino]methyl]phenyl]methyl]-3,4-diacetylaminopyrrolidine; 8-[[4-[[(2-pyridinylmethyl)amino]methyl]phenyl]methyl]-2,5,8-triaza-3-oxabicyclo [4.3.0]nonane; and 8-[[4-[[(2-pyridinylmethyl)amino]methyl]phenyl]methyl]-2,5,8-triazabicyclo[4.3.0]nonane.

Additional CXCR4 antagonists that may be used to in conjunction with the compositions and methods described herein include those described in WO 2001/085196, WO 1999/050461, WO 2001/094420, and WO 2003/090512, the disclosures of each of which are incorporated herein by reference as they pertain to compounds that inhibit CXCR4 activity or expression.

Additional CXCR4 antagonists that may be used to in conjunction with the compositions and methods described herein include those described in WO 2015/063768, for example, analog 4F-benzoyl TN14003 (4F-benzoyl-Arg-Arg-Nal-Cys-Tyr-Cit-Lys-DLys-Pro-Tyr-Arg-Cit-Cys-Arg-NH₂; wherein Nal=naphthylalanine, Cit=citrulline, DLys=D-lysine), also known as BL-8040 (BioLineRx, Modi'in, Israel).

Additional CXCR4 antagonists that may be used to in conjunction with the compositions and methods described herein include anti-CXCR4 antibodies (including modified forms of antibodies fragments, as described above). Anti-CXCR4 antibodies that may be used to in conjunction with the compositions and methods described herein include ulocuplumab (F7 in WO 2008/060367; also referred to as BMS-936564 or MDX-1338; Bristol-Myers Squibb), and the antibodies, including modified forms and fragments, provided in TABLE 3.

TABLE 3
Exemplary Anti-CXCR4 Antibodies

| Antibody (Company) | Format | References |
|---|---|---|
| Ulocuplumab (Bristol- | Human IgG4 | (Kashyap et al. (2016) Oncotarget |
| Myers Squibb) | | 7(3): 2809-2822; Kuhne et al. (2013) Clin |
| | | Cancer Res 19(2): 357-366) |
| LY2624587 (Eli Lilly and | Humanized | (Peng et al. (2017) Oncotarget 8(55): 94619- |
| Company) | IgG4 | 94634; Peng et al. (2016) PLoS One 11(3): |
| | | e0150585) |
| PF-06747143 (Pfizer) | Humanized | (Kashyap et al. (2017) J Hematol Oncol |
| | IgG1 | 10(1): 112; Liu et al. (2017) Blood Advances |
| | | 1(15): 1088-1100; Zhang et al. (2017) Sci |
| | | Rep 7(1): 7305) |
| hz515H7/F50067 (Pierre | Humanized | (Broussas et al. (2016) Mol Cancer Ther |
| Fabre) | IgG1 | 15(8): 1890-1899; Fouquet et al. (2018) |
| | | Oncotarget 9(35): 23890- 23899) |
| MEDI3185 (Medimmune) | hIgG1 triple | (Kamal et al. (2013) Cancer Research 73(8 |
| | mutant | Supplement): 5462-5462; Peng et al. (2016a) |
| | lacking | MAbs 8(1): 163-175; Schwickart et al. |
| | ADCC and | (2016) Cytometry B Clin Cytom 90(2): 209- |
| | CDC | 219) |
| IgGX-auristatin | IgG antibody- | (Kularatne et al., (2014) Angewandte Chemie |
| | drug | (International ed in English) 53(44): 11863- |
| | conjugate | 11867) |
| 238D2, 238D4 (Ablynx) | Nanobody | (Jahnichen et al. (2010) Proc Natl Acad Sci |
| | | USA 107(47): 20565-20570) |
| 10A10 | Nanobody | (de Wit et al., (2017) J Pharmacol Exp Ther |
| | | 363(1): 35-44) |
| VUN400-402 | Nanobody | (Van Hout et al. (2018) Biochem Pharmacol. |
| | | 158: 402-412) |
| VUN400-402 | Nanobody | (Bobkov et al. (2018b) 158: 413-424) |
| | fused with Fc | |
| | domain from | |
| | IgG1 | |
| AD-114 (AdAlta) | i-body | (Griffiths et al. (2016) J Biol Chem 291(24): |
| | | 12641-12657; Griffiths et al., 2018 Sci Rep |
| | | 8(1): 3212) |
| bAb-AC1, bAb-AC4 | Antibody-like | (Liu et al. (2014) J Am Chem Soc 136(30): |
| | scaffold | 10557-10560) |
| | protein | |

### Methods for the Recombinant Expression of Peptides and Proteins

Peptides and proteins described herein (e.g., CXCR2 agonists, such as Gro-β, Gro-β T, Gro-β N69D, Gro-β T N65D, and variants thereof) can be expressed in host cells, for example, by delivering to the host cell a nucleic acid encoding the corresponding peptide or protein. The sections that follow describe a variety of techniques that can be used for the purposes of introducing nucleic acids encoding peptides and proteins described herein to a host cell for the purposes of recombinant expression.

### Transfection Techniques

Techniques that can be used to introduce a polynucleotide, such as nucleic acid encoding a CXCR2 agonist, such as Gro-β, Gro-β T, Gro-β N69D, Gro-β T N65D, or a variant thereof, into a cell (e.g., a mammalian cell, such as a human cell) are known in the art. In some embodiments, electroporation can be used to permeabilize mammalian cells (e.g., human cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al. (1987) Nucleic Acids Research 15:1311, the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al. (2005) Experimental Dermatology 14:315, as well as in U.S. 2010/0317114, the disclosures of each of which are incorporated herein by reference.

Additional techniques useful for the transfection of host cells for the purposes of recombinant peptide and protein expression include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human cell. Squeeze-poration is described in detail, e.g., in Sharei et al. (2013) Journal of Visualized Experiments 81:e50980, the disclosure of which is incorporated herein by reference.

Lipofection represents another technique useful for transfection of cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane are activated dendrimers (described, e.g., in Dennig (2003) Topics in Current Chemistry 228:227, the disclosure of which is incorporated herein by reference) and diethylaminoethyl (DEAE)-dextran, the use of which as a transfection agent is described in detail, for example, in Gulick et al. (1997) Current Protocols in Molecular Biology 40:I:9.2:9.2.1, the disclosure of which is incorporated herein by reference. Magnetic beads are another tool that can be used to transfect cells in a mild and efficient manner, as this methodology utilizes an applied magnetic field in order to direct the uptake of nucleic acids. This technology is described in detail, for example, in U.S. 2010/0227406, the disclosure of which is incorporated herein by reference.

Another useful tool for inducing the uptake of exogenous nucleic acids by cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al. (2007) Methods in Cell Biology 82:309, the disclosure of which is incorporated herein by reference.

Microvesicles represent another potential vehicle that can be used to introduce a nucleic acid encoding a peptide or protein described herein into a host cell for the purpose of recombinant expression. In some embodiments, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Viral Vectors for Nucleic Acid Delivery

Viral genomes provide a rich source of vectors that can be used for the efficient delivery of exogenous nucleic acids encoding peptides and proteins described herein, such as CXCR2 agonists, including Gro-β, Gro-β T, Gro-β N69D, Gro-β T N65D, and variants thereof, into host cells for the purpose of recombinant expression. Viral genomes are particularly useful vectors for gene delivery because the polynucleotides contained within such genomes may be incorporated into the genome of a cell, for example, by way of generalized or specialized transduction. These processes may occur as part of the natural replication cycle of a viral vector, and may not require added proteins or reagents in order to induce gene integration. Examples of viral vectors that may be used to introduce a nucleic acid molecule encoding a peptide or protein described herein into a host cell for recombinant expression include parvovirus, such as adeno-associated virus (AAV), retrovirus, adenovirus (e.g., Ad5, Ad26, Ad34, Ad35, and Ad48), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding peptides and proteins described herein to host cells for recombinant expression purposes include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene delivery and recombinant protein and peptide expression.

### Methods of Treatment

As described herein, hematopoietic stem cell transplant therapy can be administered to a subject in need of treatment so as to populate or repopulate one or more blood cell types, such as a blood cell lineage that is deficient or defective in a patient suffering from a stem cell disorder. Hematopoietic stem and progenitor cells exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis. Thus, hematopoietic stem and progenitor cells represent a useful therapeutic modality for the treatment of a wide array of disorders in which a patient has a deficiency or defect in a cell type of the hematopoietic lineage. The deficiency or defect may be caused, for example, by depletion of a population of endogenous cells of the hematopoietic system due to administration of a chemotherapeutic agent (e.g., in the case of a patient suffering from a cancer, such as a hematologic cancer described herein). The deficiency or defect may be caused, for example, by depletion of a population of endogenous hematopoietic cells due to the activity of self-reactive immune cells, such as T lymphocytes or B lymphocytes that cross-react with self antigens (e.g., in the case of a patient suffering from an autoimmune disorder, such as an autoimmune disorder described herein). Additionally or alternatively, the deficiency or defect in cellular activity may be caused by aberrant expression of an enzyme (e.g., in the case of a patient suffering from various metabolic disorders, such as a metabolic disorder described herein).

Thus, hematopoietic stem cells can be administered to a patient defective or deficient in one or more cell types of the hematopoietic lineage in order to re-constitute the defective or deficient population of cells in vivo, thereby treating the pathology associated with the defect or depletion in the endogenous blood cell population. Hematopoietic stem and progenitor cells can be used to treat, e.g., a non-malignant hemoglobinopathy (e.g., a hemoglobinopathy selected from the group consisting of sickle cell anemia, thalassemia, Fanconi anemia, aplastic anemia, and Wiskott-Aldrich syndrome). In these cases, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of cells that are damaged or deficient in the patient.

Additionally or alternatively, hematopoietic stem and progenitor cells can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). In these cases, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of immune cells (e.g., T lymphocytes, B lymphocytes, NK cells, or other immune cells) that are damaged or deficient in the patient.

Hematopoietic stem and progenitor cells can also be used to treat a metabolic disorder (e.g., a metabolic disorder selected from the group consisting of glycogen storage diseases, mucopolysaccharidoses, Gaucher Disease, Hurler Disease, sphingolipidoses, metachromatic leukodystrophy, globoid cell leukodystrophy, and cerebral adrenoleukodystrophy). In these cases, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of hematopoietic cells that are damaged or deficient in the patient.

Additionally or alternatively, hematopoietic stem or progenitor cells can be used to treat a malignancy or proliferative disorder, such as a hematologic cancer or myeloproliferative disease. In the case of cancer treatment, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of cells that are damaged or deficient in the patient, such as a population of hematopoietic cells that is damaged or deficient due to the administration of one or more chemotherapeutic agents to the patient. In some embodiments, hematopoietic stem or progenitor cells may be infused into a patient in order to repopulate a population of cells depleted during cancer cell eradication, such as during systemic chemotherapy. Exemplary hematological cancers that can be treated by way of administration of hematopoietic stem and progenitor cells in accordance with the compositions and methods described herein are acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

In various embodiments, hematopoietic stem or progenitor cells can be mobilized from a donor previously diagnosed with a malignancy or proliferative disorder. The hematopoietic stem and progenitor cells thus mobilized may then be withdrawn from the donor and administered to a patient. In various embodiments, the donor and patient are different individuals and therefore, the administration of the hematopoietic stem and progenitor cells represents an allogeneic transplant. In various embodiments, the donor and patient are the same individual and therefore, the administration of the hematopoietic stem and progenitor cells represents an autologous transplant. In particular embodiments, the donor was previously diagnosed with a hematological malignancy, such as multiple myeloma. Thus, for an autologous transplant, the donor (e.g., a patient having a hematological malignancy) is administered a CXCR4 antagonist and/or a CXCR2 agonist to mobilize hematopoietic stem or progenitor cells. The mobilized hematopoietic stem and progenitor cells are then administered to the donor (e.g., the patient having a hematological malignancy) as an autologous transplant.

For example, a donor (e.g., a patient having a hematological malignancy) may be administered a CXCR4 antagonist and/or a CXCR2 agonist to mobilize hematopoietic stem or progenitor cells into the peripheral blood of the donor. Following mobilization of hematopoietic stem or progenitor cells in the donor (e.g., a patient having a hematological malignancy), the mobilized hematopoietic stem or progenitor cells may be collected (e.g., by apheresis) and stored for later use. Subsequently, the donor may be administered a treatment (for example, chemotherapy, e.g., high-dose chemotherapy, radiation therapy, e.g., total body irradiation, and combinations thereof) for an underlying disease (for example, a hematological malignancy, e.g., multiple myeloma). The donor may then be administered the donor's stored hematopoietic stem or progenitor cells as part of hematopoietic stem cell transplant therapy.

In certain embodiments, the disclosure relates to a method of mobilizing a population of hematopoietic stem or progenitor cells from the bone marrow of a mammalian donor, e.g., a donor with multiple myeloma, into peripheral blood, the method comprising administering to the donor a CXCR2 agonist selected from the group consisting of Gro-β, Gro-β T, and variants thereof at a dose of from about 0.001 mg/kg to about 0.1 mg/kg. In certain embodiments, the Gro-β, Gro-β T, and variants thereof is administered at a dose of from about 0.03 mg/kg. In various embodiments, the CXCR2 agonist selected from the group consisting of Gro-β, Gro-β T, and variants thereof can be administered over a period of time of less than about 15 minutes or a period of time of less than about 10 minutes, for example, from about 1 to about 15 minutes, from about from about 1 to about 10 minutes, from about 1 to about 9 minutes, from about from about 1 to about 8 minutes, from about 1 to about 7 minutes, from about from about 1 to about 6 minutes, from about 1 to about 5 minutes, from about 1 to about 4 minutes, from about from about 1 to about 3 minutes, from about from about 1 to about 2 minutes, from about 2 to about 15 minutes, from about 2 to about 10 minutes, from about 2 to about 9 minutes, from about 2 to about 8 minutes, from about 2 to about 7 minutes, from about 2 to about 6 minutes, from about 2 to about 5 minutes, from about 2 to about 4 minutes, from about 2 to about 3 minutes, from about 3 to about 15 minutes, from about 3 to about 10 minutes, from about 3 to about 9 minutes, from about 3 to about 8 minutes, from about 3 to about 7 minutes, from about 3 to about 6 minutes, from about 3 to about 5 minutes, from about 3 to about 4 minutes, from about 4 to about 15 minutes, from about 4 to about 10 minutes, from about 4 to about 9 minutes, from about 4 to about 8 minutes, from about 4 to about 7 minutes, from about 4 to about 6 minutes, or from about 4 to about 5 minutes. In particular embodiments, the CXCR2 agonist selected from the group consisting of Gro-β, Gro-β T, and variants thereof can be administered over a period of time of from about 3 to about 10 minutes. In certain embodiments, a CXCR4 antagonist, such as plerixafor is administered, for example, at a dosage of about 160 μg/kg or about 240 g/kg.

In certain embodiments, the donor, e.g., the donor with multiple myeloma, experiences different severity of adverse effects depending on the period of time that the CXCR2 agonist is administered. In particular embodiments, the donor that receives a shorter administration of a CXCR2 agonist experiences less severe adverse effects in comparison to a donor that receives a longer administration of a CXCR2 agonist. In various embodiments, a donor that receives administration of a CXCR2 agonist over a period of time of less than about 10 minutes experiences fewer or less severe adverse effects in comparison to a donor who receives administration of the CXCR2 agonist over a period of time greater than about 10 minutes. In particular embodiments, a donor that receives administration of a CXCR2 agonist over a period of time of less than about 20 minutes experiences fewer or less severe adverse effects in comparison to a donor who receives administration of the CXCR2 agonist over a period of time greater than about 20 minutes. In various embodiments, a donor that receives administration of a CXCR2 agonist over a period of time of less than about 30 minutes experiences fewer or less severe adverse effects in comparison to a donor who receives administration of the CXCR2 agonist over a period of time greater than about 30 minutes. In various embodiments, a donor that receives administration of a CXCR2 agonist over a period of time of less than about 40 minutes experiences fewer or less severe adverse effects in comparison to a donor who receives administration of the CXCR2 agonist over a period of time greater than about 40 minutes. In various embodiments, a donor that receives administration of a CXCR2 agonist over a period of time of less than about 50 minutes experiences fewer or less severe adverse effects in comparison to a donor who receives administration of the CXCR2 agonist over a period of time greater than about 50 minutes. In various embodiments, a donor that receives administration of a CXCR2 agonist over a period of time of less than about 60 minutes experiences fewer or less severe adverse effects in comparison to a donor who receives administration of the CXCR2 agonist over a period of time greater than about 60 minutes.

The instant disclosure relates in part to the surprising discovery that administration of a CXCR2 agonist (e.g., Gro-β, Gro-β T, and variants thereof) allows for the collection of a sufficient number of CD34⁺ cells for a transplant in a multiple myeloma patient, for example, after a single dose and/or a single-day mobilization regimen. In particular embodiments, CD34+ cells may be collected in an amount of at least about 2×10⁶ cells/kg of body weight from a patient. In particular embodiments, CD34+ cells may be collected in an amount of at least about 4×10⁶ cells/kg of body weight from a patient. In particular embodiments, CD34+ cells may be collected in an amount of at least about 6×10⁶ cells/kg of body weight from a patient. In some embodiments, the CD34+ cells may be mobilized and collected in a single day. In certain embodiments, the CD34+ cells may be mobilized and collected over the course of two or more days. For example, in some embodiments, the CD34+ cells may be mobilized and collected on two consecutive days.

In certain embodiments, the multiple myeloma patient, who can be the donor and/or recipient, has undergone an induction therapy prior to mobilization. Induction therapy is performed to reduce the number of myeloma cells (plasma cells) in the bone marrow. In certain embodiments, the multiple myeloma patient has undergone an induction therapy for about 1 to about 6 months (e.g., for about 3 to about 6 months, or for about 4 months) prior to mobilization of hematopoietic stem and progenitor cells according to the methods described herein. In certain embodiments, the induction therapy comprises a CyBorD regimen (cyclophosphamide (Cytoxan®, Procytox®), bortezomib (Velcade®) and dexamethasone (Decadron®, Dexasone®)); a VRD regimen (bortezomib, lenalidomide (Revlimid®) and dexamethasone; thalidomide (Thalomid®) and dexamethasone; lenalidomide and low-dose dexamethasone; bortezomib and dexamethasone; a VTD regimen (bortezomib, thalidomide and dexamethasone); bortezomib, cyclophosphamide and prednisone; bortezomib, doxorubicin (Adriamycin®) and dexamethasone; dexamethasone, or liposomal doxorubicin (Caelyx®, Doxil®), vincristine (Oncovin®) and dexamethasone. In certain embodiments, the induction therapy is VTD. In certain embodiments, the induction therapy is VTD and daratumumab. In certain embodiments, the multiple myeloma patient has received about 4-6 cycles (e.g., about 6 cycles) of lenalidomide. In certain embodiments, following induction therapy, the multiple myeloma patient has been evaluated as having a very good partial response (VGPR). VGPR can be determined by the presence of one or more of the following: serum and urine M-protein detectable by immunofixation but not on electrophoresis; ≥90% reduction in serum M-protein and urine M-protein level <100 mg/24 hours; and ≥90% decrease in the difference between involved and uninvolved free light chain levels (applicable to Light Chain Only Myeloma).

In certain embodiments, the multiple myeloma patient has stage I, stage II, or stage III multiple myeloma. In certain embodiments, the multiple myeloma patient has high risk fluorescence in situ hybridization (FISH), for example, based on the detection of one or more cytogenetic abnormalities such as t(4;14), del(17/17p), t(14;16), t(14;20), nonhyperdiploidy, and gain (1q). (See, Sonneveld et al. (2016) Blood 126(24):2955-2962.)

In certain embodiments, the patient (e.g., the multiple myeloma patient) has at least one risk factor for poor mobilization. In certain embodiments, the risk factor is a prior other cancer treated with chemotherapy and/or radiation, an autoimmune disorder needing systemic therapy, treatment with daratumumab, or alkylator exposure for more than 2 months.

In various embodiments, hematopoietic stem and progenitor cells are administered to a recipient with multiple myeloma in combination with an additional therapeutic agent. In various embodiments, the additional therapeutic agent is a multiple myeloma therapeutic agent. In instances where the additional therapeutic agent is cytotoxic to hematopoietic stem and progenitor cells, it may be necessary to delay administration of the hematopoietic stem and progenitor cells until after the additional therapeutic agent has cleared the recipient (e.g., completely or to a non-cytotoxic level). As an example, the additional therapeutic agent may be melphalan. In various embodiments, melphalan is administered to the recipient at a dose of about 100 mg/m² to about 500 mg/m². In various embodiments, melphalan is administered to the recipient at a dose of about 100 mg/m² to about 200 mg/m². In various embodiments, melphalan is administered to the recipient at a dose of about 140 mg/m² to about 200 mg/m2. In various embodiments, melphalan is administered to the recipient at a dose of about 150 mg/m² to about 400 mg/m². In various embodiments, melphalan is administered to the recipient at a dose of about 200 mg/m² to about 300 mg/m². In various embodiments, melphalan is administered to the recipient at a dose of about 200 mg/m². Subsequently (e.g., after the melphalan has cleared the recipient), the recipient is administered the hematopoietic stem and progenitor cells.

Hematopoietic stem or progenitor cells mobilized to the peripheral blood of a subject may be withdrawn (e.g., harvested or collected) from the subject by any suitable technique. For example, the hematopoietic stem or progenitor cells may be withdrawn by a blood draw. In some embodiments, hematopoietic stem or progenitor cells mobilized to a subject's peripheral blood as contemplated herein may be harvested (i.e., collected) using apheresis. In some embodiments, apheresis may be used to enrich a donor's blood with mobilized hematopoietic stem or progenitor cells.

Additional diseases that can be treated by the administration of hematopoietic stem and progenitor cells to a patient include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

In addition, administration of hematopoietic stem and progenitor cells can be used to treat autoimmune disorders. In certain embodiments, mobilization of hematopoietic stem and progenitor cells in a subject with an autoimmune disorder using the methods disclosed herein lessens or avoids the autoimmune disorder flares that can occur during mobilization with G-CSF. Accordingly, provided herein is a method of mobilizing HSCs in a patient having an autoimmune disorder, e.g., multiple sclerosis, by administering a CXCR2 agonist, optionally in combination with a CXCR4 antagonist, wherein the risk of inducing a flare of the patient's autoimmune disorder is reduced or eliminated, e.g., as compared to mobilizing HSCs in a patient having an autoimmune disorder by administering G-CSF.

In some embodiments, upon infusion into a patient, transplanted hematopoietic stem and progenitor cells may home to a stem cell niche, such as the bone marrow, and establish productive hematopoiesis. This, in turn, can re-constitute a population of cells depleted during autoimmune cell eradication, which may occur due to the activity of self-reactive lymphocytes (e.g., self-reactive T lymphocytes and/or self-reactive B lymphocytes). Autoimmune diseases that can be treated by way of administering hematopoietic stem and progenitor cells to a patient include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatricial pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

In some embodiments, a method of harvesting hematopoietic stem cells from a human subject is provided. The method comprises administering a CXCR2 agonist and a CXCR4 antagonist to the human subject and harvesting the hematopoietic stem cells from peripheral blood of the human subject.

In some embodiments, a method of transplanting hematopoietic stem cells into a human patient in need thereof is provided. The method comprises administering a CXCR2 agonist and optionally a CXCR4 antagonist to a hematopoietic stem cell donor, harvesting the hematopoietic stem cells from peripheral blood of the donor, and transplanting the harvested hematopoietic stem cells into the patient.

In addition, the disclosure relates to a method of preventing, reducing the risk of developing, or reducing the severity of graft versus host disease (GVHD) in a patient in need thereof, wherein the method includes infusing into the patient a therapeutically effective amount of hematopoietic stem cells, wherein the hematopoietic stem cells were mobilized from bone marrow of a mammalian donor into peripheral blood by the methods described herein, e.g., including administering to the mammalian donor a CXCR2 agonist and a CXCR4 antagonist.

For example, in some embodiments, a method of transplanting hematopoietic stem cells into a human patient in need thereof is provided, where the method prevents, reduces the risk of developing, or reduces the severity of graft versus host disease (GVHD) in the patient, wherein the method includes infusing into the patient a therapeutically effective amount of hematopoietic stem cells, wherein the hematopoietic stem cells were mobilized from bone marrow of a mammalian donor into peripheral blood by the methods described herein, e.g., including administering to the mammalian donor a CXCR2 agonist and a CXCR4 antagonist. In certain embodiments, the hematopoietic stem cells infused into the patient were obtained from the donor by apheresis. (e.g., leukapheresis) after being mobilized into the peripheral blood of the donor.

In certain embodiments, the number and/or proportion of CD8+ T-cells mobilized by the methods described herein is less than the number and/or proportion of CD8+ T-cells mobilized using G-CSF or plerixafor alone. For example, the number of CD8+ T-cell mobilized can be from about 0 to about 0.6×10⁸/kg (e.g., from about 0 to about 1×10⁸/kg, about 0 to about 2×10⁸/kg, about 0 to about 3×10⁸/kg, about 0 to about 4×10⁸/kg, about 0 to about 5×10⁸/kg, about 1×10⁸/kg to about 2×10⁸/kg, about 1×10⁸/kg to about 3×10⁸/kg, about 1×10⁸/kg to about 4×10⁸/kg, about 1×10⁸/kg to about 5×10⁸/kg, about 1×10⁸/kg to about 6×10⁸/kg, about 2×10⁸/kg to about 3×10⁸/kg, about 2×10⁸/kg to about 4×10⁸/kg, about 2×10⁸/kg to about 5×10⁸/kg, about 2×10⁸/kg to about 6×10⁸/kg, about 3×10⁸/kg to about 4×10⁸/kg, about 3×10⁸/kg to about 5, about 3×10⁸/kg to about 6×10⁸/kg, about 4×10⁸/kg to about 5×10⁸/kg, about 4×10⁸/kg to about 6×10⁸/kg, or about 5×10⁸/kg to about 6×10⁸/kg). In certain embodiments, CD8+ T-cells constitute from about 0.5 to about 5% of the graft (e.g., from about 0.5% to about 1%, about 0.5% to about 2%, about 0.5% to about 3%, about 0.5% to about 4%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%).

### Methods of Treating Neutropenia

The present invention further provides methods and compositions for treating neutropenia in a patient, such as a mammalian patient (e.g., a human patient) in need thereof. Patients that are “in need” of treatment include patients that have been diagnosed by a physician as having neutropenia, e.g., exhibiting mild neutropenia (about 1000 to about 1500 neutrophils/μl of blood); moderate neutropenia (about 500 to about 1000 neutrophils/μl of blood); or severe neutropenia (below about 500 neutrophils/μl of blood). Patients “in need” of treatment also include patients that will or are currently undergoing a therapeutic regimen expected to induce neutropenia, e.g., chemotherapy.

Using the compositions and methods described herein, a C-X-C chemokine receptor type 2 (CXCR2) agonist, such as Gro-β or a variant thereof, such as a truncated form of Gro-β (e.g., Gro-β T), as described herein, optionally in combination with a C-X-C chemokine receptor type 4 (CXCR4) antagonist, such as 1,1′-[1,4-phenylenebis(methylene)]-bis-1,4,8,11-tetra-azacyclotetradecane or a variant thereof, may be administered to a patient, as described herein, in amounts sufficient in to mobilize a population of neutrophils from the bone marrow of the patient into peripheral blood.

The invention is further based, in part, on the discovery that administration of a surprisingly low dose of a CXCR2 agonist, such as Gro-β, Gro-β T, or a variant thereof, optionally in combination with a CXCR4 antagonist, such as plerixafor or a pharmaceutically acceptable salt thereof, at particular doses can provide the important clinical benefit of mobilizing neutrophils.

The CXCR4 antagonists and CXCR2 agonists described herein, using the methods described herein for the mobilization of stem and progenitor cells, likewise can be administered to a patient so as to induce mobilization of a population of neutrophils from bone marrow into peripheral blood, thereby to treat neutropenia.

In some embodiments, administration of a CXCR2 agonist, optionally in combination with a CXCR4 antagonist, may decrease the incidence of infection (e.g., as manifested by febrile neutropenia) in patients with cancer (e.g., nonmyeloid malignancies receiving) anticancer drugs (e.g., myelosuppressive drugs) associated with neutropenia (e.g., a significant incidence of severe neutropenia).

In some embodiments, administration of a CXCR2 agonist, optionally in combination with a CXCR4 antagonist, may reduce the time to neutrophil recovery following chemotherapy treatment (e.g., induction or consolidation chemotherapy) of patients suffering from cancer (e.g., a blood cancer, e.g., acute myeloid leukemia). In some embodiments, administration of a CXCR2 agonist, optionally in combination with a CXCR4 antagonist, may reduce the duration of fever following chemotherapy treatment (e.g., induction or consolidation chemotherapy) of patients suffering from cancer (e.g., a blood cancer, e.g., acute myeloid leukemia). In certain embodiments, a CXCR2 agonist, optionally in combination with a CXCR4 antagonist, may be administered to a patient in combination with G-CSF to reduce the number of days to neutrophil recovery.

In some embodiments, administration of a CXCR2 agonist, optionally in combination with a CXCR4 antagonist, may reduce the duration of neutropenia and neutropenia-related clinical sequelae, e.g., febrile neutropenia, in patients with nonmyeloid malignancies undergoing myeloablative chemotherapy followed by hematopoietic stem cell transplantation.

In some embodiments, administration of a CXCR2 agonist, optionally in combination with a CXCR4 antagonist, may reduce the incidence and duration of sequelae of neutropenia (e.g., fever, infections, oropharyngeal ulcers) in symptomatic patients with congenital neutropenia, cyclic neutropenia, or idiopathic neutropenia.

As used herein with respect to neutropenia, the term “mobilizing amount” refers to a quantity of one or more agents, such as a quantity of a CXCR4 antagonist and/or a CXCR2 agonist described herein (In some embodiments, a quantity of plerixafor, or a variant thereof, and/or Gro-β, or a variant thereof, such as a truncation of Gro-β, for example, Gro-β T) that mobilizes a population of neutrophils upon administration to a subject, such as a mammalian subject (e.g., a human subject). Exemplary mobilizing amounts of these agents include amounts sufficient to effectuate the release of a population of, for example, from about 5×10³ to about 20×10³ neutrophils/μL of peripheral blood, such as from about 5 to about 8 neutrophils/μL of peripheral blood, about 5 to about 10 neutrophils/μL of peripheral blood, about 5 to about 12 neutrophils/μL of peripheral blood, about 5 to about 15 neutrophils/μL of peripheral blood, about 5 to about 18 neutrophils/μL of peripheral blood, about 8 to about 10 neutrophils/μL of peripheral blood, about 8 to about 12 neutrophils/μL of peripheral blood, about 8 to about 15 neutrophils/μL of peripheral blood, or about 8 to about 18 neutrophils/μL of peripheral blood, about 8 to about 20 neutrophils/μL of peripheral blood, about 10 to about 12 neutrophils/μL of peripheral blood, about 10 to about 15 neutrophils/μL of peripheral blood, about 10 to about 18 neutrophils/μL of peripheral blood, about 10 to about 20 neutrophils/μL of peripheral blood, about 12 to about 15 neutrophils/μL of peripheral blood, about 10 to about 18 neutrophils/μL of peripheral blood, about 10 to about 20 neutrophils/μL of peripheral blood, about 12 to about 15 neutrophils/μL of peripheral blood, about 12 to about 18 neutrophils/μL of peripheral blood, about 12 to about 20 neutrophils/μL of peripheral blood, about 15 to about 18 neutrophils/μL of peripheral blood, or about 15 to about 20 neutrophils/μL of peripheral blood.

### Selection of Donors and Patients

In some embodiments, the patient is the donor. In such cases, withdrawn hematopoietic stem or progenitor cells may be re-infused into the patient, such that the cells may subsequently home hematopoietic tissue and establish productive hematopoiesis, thereby populating or repopulating a line of cells that is defective or deficient in the patient (e.g., a population of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes). In this scenario, the transplanted hematopoietic stem or progenitor cells are least likely to undergo graft rejection, as the infused cells are derived from the patient and express the same HLA class I and class II antigens as expressed by the patient.

Alternatively, the patient and the donor may be distinct. In some embodiments, the patient and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs).

In some embodiments, the patient and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

In certain embodiments, the donor has a mild or moderate reduction in glomerular filtration rate (GFR). Patients with chronic kidney disease are classified into stages based upon their glomerular filtration rate (GFR). The GFR for stage 1 patients is ≥90 mL/minute/1.73 m² (considered normal), stage 2 is 60-89 mL/minute/1.73 m² (considered mild), stage 3 is 30-59 mL/minute/1.73 m² (considered mild to moderate), stage 4 is 15-29 mL/minute/1.73 m² (considered severe), and stage 5 is <15 mL/minute/1.73 m² (kidney failure).

### Methods of Genetic Modification of Hematopoietic Stem and Progenitor Cells

Prior to infusion into a patient, such as a patient having one or more stem cell disorders described herein, hematopoietic stem cells obtained from a donor (or progeny thereof) may be genetically modified, for example, by editing (e.g., correcting, disrupting, etc.) an endogenous gene. This strategy can be used, for example, to silence the expression of one or more major histocompatibility complex genes in a hematopoietic stem cell that is allogeneic with respect to the patient, thereby reducing the likelihood of graft rejection upon transplantation.

A wide array of methods has been established for the disruption of target genes in a population of cells. In some embodiments, one such method is through the use of a clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific Cas9-mediated DNA cleavage can be engendered in a foreign polynucleotide because the interaction that brings Cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al. (2013) Nature Biotechnology 31:227, the disclosure of which is incorporated herein by reference) and can be used as an efficient means of site-specifically editing hematopoietic stem cell genomes in order to cleave DNA, for example, prior to the incorporation of a gene encoding a target protein. The use of CRISPR/Cas to modulate gene expression has been described in, e.g., U.S. Pat. No. 8,697,359, the disclosure of which is incorporated herein by reference. Alternative methods for site-specifically cleaving genomic DNA prior to the incorporation of a gene of interest in a hematopoietic stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. (2010) Nature Reviews Genetics 11:636; and in Joung et al. (2013) Nature Reviews Molecular Cell Biology 14:49, the disclosure of both of which are incorporated herein by reference.

Additional genome editing techniques that can be used to incorporate polynucleotides encoding target genes into the genome of a hematopoietic stem cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a hematopoietic stem cell. These single-chain nucleases have been described extensively in, e.g., U.S. Pat. Nos. 8,021,867 and 8,445,251, the disclosures of each of which are incorporated herein by reference.

### Expansion of Hematopoietic Stem and Progenitor Cells

Prior to infusion into a patient, hematopoietic stem and progenitor cells may be expanded ex vivo, for example, by contacting the cells with an aryl hydrocarbon receptor antagonist. Aryl hydrocarbon receptor antagonists useful in conjunction with the compositions and methods described herein include those described in U.S. Pat. Nos. 9,580,426 and 10,351,572, the disclosures of each of which are incorporated herein by reference in their entirety.

In some embodiments, hematopoietic stem and progenitor cells mobilized by the methods contemplated herein and collected (e.g., by apheresis) from a patient for use in an autologous transplant may be expanded (e.g., by culturing the collected, mobilized hematopoietic stem and progenitor cells in the presence of an aryl hydrocarbon receptor antagonist) and subsequently infused into the same patient.

In some embodiments, hematopoietic stem and progenitor cells mobilized by the methods contemplated herein and collected (e.g., by apheresis) from a donor for use in an allogeneic transplant may be expanded (e.g., by culturing the collected, mobilized hematopoietic stem and progenitor cells in the presence of an aryl hydrocarbon receptor antagonist) and subsequently infused into a recipient patient. For example, in instances where additional hematopoietic stem and progenitor cells are desired from a particular hematopoietic stem cell donor, the mobilized hematopoietic stem and progenitor cells collected from the donor may be expanded to yield a higher dose of mobilized hematopoietic stem and progenitor cells for a recipient.

In some embodiments, aryl hydrocarbon receptor antagonists include those represented by formula (III)

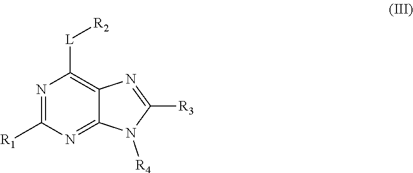

in which:
L is selected from —NR₅ₐ(CH₂)₂₋₃, —NR₅ₐ(CH₂)₂NR_{5b}—, —NR₅ₐ(CH₂)₂S—, NR₅ₐCH₂CH(OH)— and —NR₅ₐCH(CH₃)CH₂—; wherein R₅ₐ and R_{5b} are independently selected from hydrogen and C₁₋₄ alkyl;
R₁ is selected from thiophenyl, 1H-benzoimidazolyl, isoquinolinyl, 1H-imidazopyridinyl, benzothiophenyl, pyrimidinyl, pyridinyl, pyrazinyl, pyridazinyl, and thiazolyl; In some embodiments, wherein the thiophenyl, 1H-benzoimidazolyl, isoquinolinyl, 1H-imidazopyridinyl, benzothiophenyl, pyrimidinyl, pyridinyl, pyrazinyl, pyridazinyl, or thiazolyl of R₁ can be optionally substituted by 1 to 3 radicals independently selected from cyano, hydroxy, C₁₋₄ alkyl, C₁₋₄ alkoxy, halo, halo-substituted-C₁₋₄ alkyl, halo-substituted-C₁₋₄alkoxy, amino, —C(O)R₈ₐ, —S(O)₀₋₂R₈ₐ, —C(O)OR₈ₐ and —C(O)NR₈ₐR_{8b}; wherein R₈ₐ and R_{8b} are independently selected from hydrogen and C₁₋₄alkyl;
R₂ is selected from —S(O)₂NR₆ₐR_{6b}, —NR₆ₐC(O)R_{6b}—, —NR₆ₐC(O)NR_{6b}R_{6c}, phenyl, 1H-pyrrolopyridin-3-yl, 1H-pyrrolopyridin-5-yl, 1H-indolyl thiophenyl, pyridinyl, 1H-1,2,4-triazolyl, 2-oxoimidazolidinyl, 1H-pyrazolyl, 2-oxo-2,3-dihydro-1H-benzoimidazolyl and 1H-indazolyl; wherein R₆ₐ, R_{6b} and R_{6c} are independently selected from hydrogen and C₁₋₄alkyl; and the phenyl, 1H-pyrrolopyridin-3-yl, 1H-pyrrolo[2,3-b]pyridin-5-yl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl or 1H-indazolyl of R₂ is optionally substituted with 1 to 3 radicals independently selected from hydroxy, halo, methyl, methoxy, amino, —O(CH₂)₂NR₇ₐR_{7b}, —S(O)₂NR₇ₐR_{7b}, —OS(O)₂NR₇ₐR_{7b} and —NR₇ₐS(O)₂R_{7b}; wherein R₇ₐ and R_{7b} are independently selected from hydrogen and C₁₋₄ alkyl;
R₃ is selected from hydrogen, C₁₋₄ alkyl and biphenyl; and
R₄ is selected from C₁₋₁₀ alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, and benzyl, (4-pentylphenyl)(phenyl)methyl and 1-(1-(2-oxo-6,9,12-trioxa-3-azatetradecan-14-yl)-1H-1,2,3-triazol-4-yl)ethyl wherein said alkyl, cyclopropyl, cyclohexyl, 2-(2-oxopyrrolidin-1-yl)ethyl, oxetan-3-yl, oxetan-2-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl or 1-(1-(2-oxo-6,9,12-trioxa-3-azatetradecan-14-yl)-1H-1,2,3-triazol-4-yl)ethyl can be optionally substituted with 1 to 3 radicals independently selected from hydroxy, C₁₋₄alkyl and halo-substituted-C₁₋₄alkyl; or a salt thereof.

In some embodiments, aryl hydrocarbon receptor antagonists useful in conjunction with the compositions and methods described herein include SR-1, represented by formula (1), below, or a salt thereof.

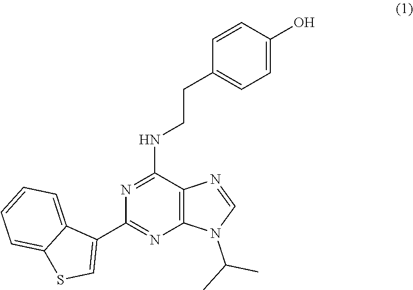 [SMILES: CC(C)n1cnc2c(NCCc3ccc(O)cc3)nc(-c3csc4ccccc34)nc21]

In some embodiments, the aryl hydrocarbon receptor antagonist is Compound 2, represented by formula (2), below, or a pharmaceutically acceptable salt, hydrate, or solvate thereof.

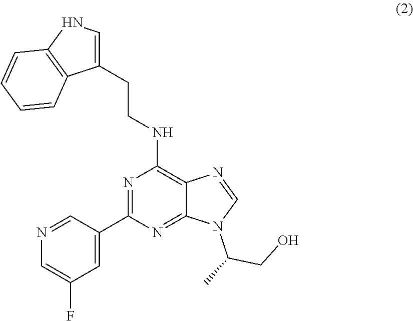 [SMILES: C[C@@H](CO)n1cnc2c(NCCc3c[nH]c4ccccc34)nc(-c3cncc(F)c3)nc21]

In some embodiments, the aryl hydrocarbon receptor antagonist is Compound 2-ent, represented by formula (2-ent), below, or a pharmaceutically acceptable salt, hydrate, or solvate thereof.

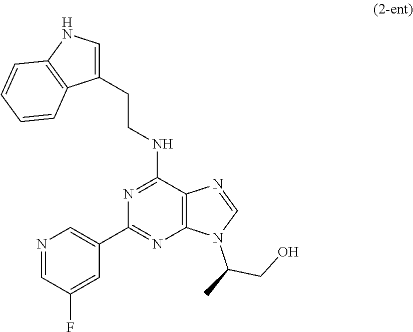 [SMILES: C[C@H](CO)n1cnc2c(NCCc3c[nH]c4ccccc34)nc(-c3cncc(F)c3)nc21]

In some embodiments, the aryl hydrocarbon receptor antagonist is Compound 2-rac, represented by formula (2-rac), below, or a pharmaceutically acceptable salt, hydrate, or solvate thereof.

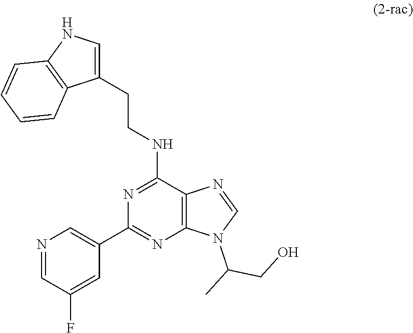 [SMILES: CC(CO)n1cnc2c(NCCc3c[nH]c4ccccc34)nc(-c3cncc(F)c3)nc21]

In some embodiments, the aryl hydrocarbon receptor antagonist is a compound represented by formula (IV) or (V)

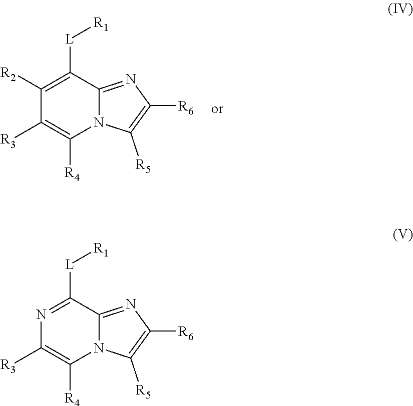

wherein:
L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, —NR₇ₐ(CR₈ₐR_{8b})ₙNR₇ₐ—, —NR₇ₐ(CR₈ₐR_{8b})ₙO—, —NR₇ₐ(CR₈ₐR_{8b})ₙS—, —O(CR₈ₐR_{8b})ₙNR₇ₐ—, —O(CR₈ₐR_{8b})ₙO—, —O(CR₈ₐR_{8b})ₙS—, —S(CR₈ₐR_{8b})ₙNR₇ₐ—, —S(CR₈ₐR_{8b})ₙO—, —S(CR₈ₐR_{8b})ₙS—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C₁₋₄ alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
R₂ is selected from the group consisting of hydrogen and optionally substituted C₁₋₄ alkyl;
R₃ is selected from the group consisting of optionally substituted aryl, optionally substituted heteroaryl, optionally substituted cycloalkyl, and optionally substituted heterocycloalkyl;
R₄ is selected from the group consisting of hydrogen and optionally substituted C₁₋₄ alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl; or a salt thereof.

In some embodiments, the aryl hydrocarbon receptor antagonist is compound (3), compound (4), compound (5), compound (6), compound (7), compound (8), compound (9), compound (10), compound (11), compound (12), compound (13), compound (25), compound (27), or compound (28)

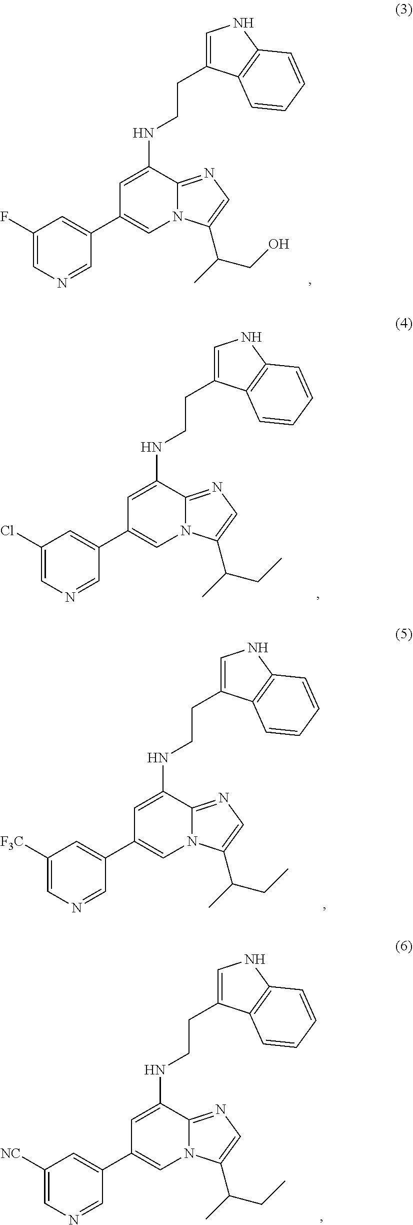 [SMILES: CC(CO)c1cnc2c(NCCc3c[nH]c4ccccc34)cc(-c3cncc(F)c3)cn12 ; CCC(C)c1cnc2c(NCCc3c[nH]c4ccccc34)cc(-c3cncc(Cl)c3)cn12 ; CCC(C)c1cnc2c(NCCc3c[nH]c4ccccc34)cc(-c3cncc(C(F)(F)F)c3)cn12 ; CCC(C)c1cnc2c(NCCc3c[nH]c4ccccc34)cc(-c3cncc(C#N)c3)cn12]

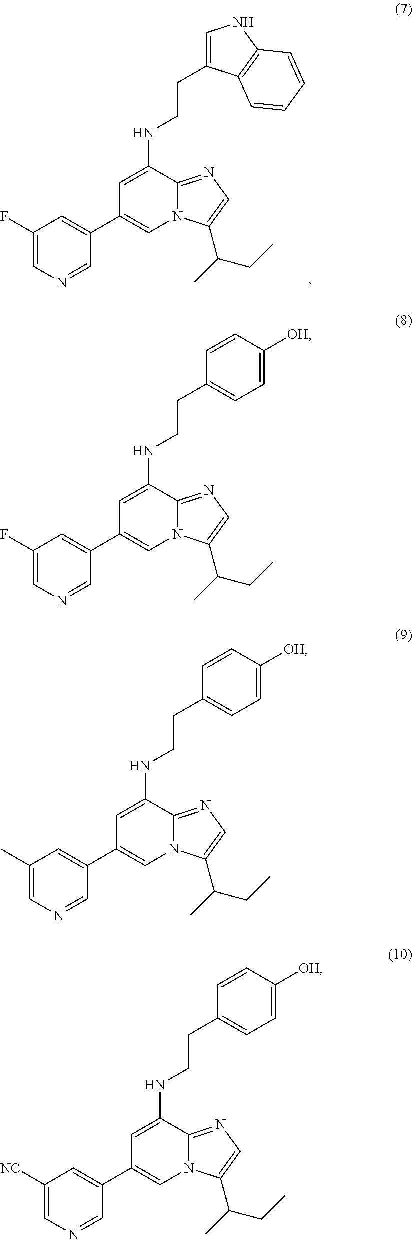 [SMILES: CCC(C)c1cnc2c(NCCc3c[nH]c4ccccc34)cc(-c3cncc(F)c3)cn12 ; CCC(C)c1cnc2c(NCCc3ccc(O)cc3)cc(-c3cncc(F)c3)cn12 ; CCC(C)c1cnc2c(NCCc3ccc(O)cc3)cc(-c3cncc(C)c3)cn12 ; CCC(C)c1cnc2c(NCCc3ccc(O)cc3)cc(-c3cncc(C#N)c3)cn12]

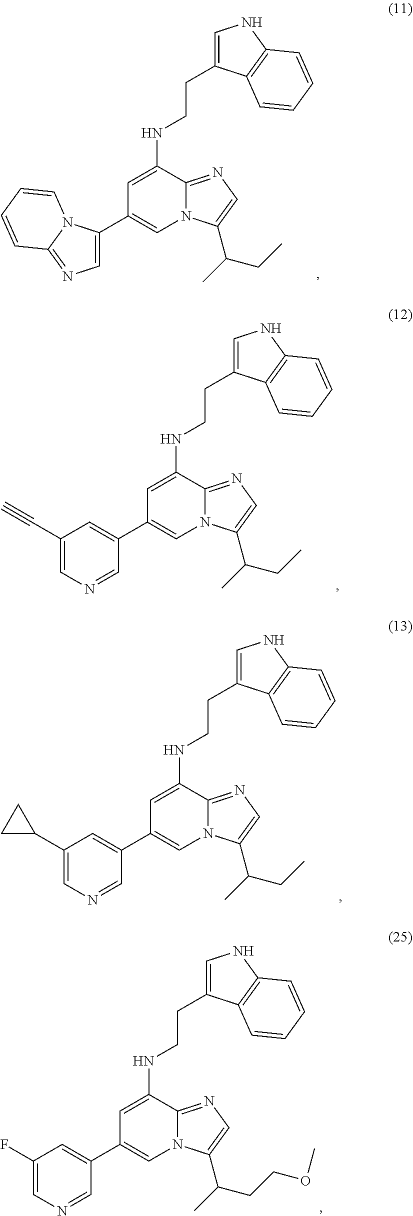 [SMILES: CCC(C)c1cnc2c(NCCc3c[nH]c4ccccc34)cc(-c3cnc4ccccn34)cn12 ; C#Cc1cncc(-c2cc(NCCc3c[nH]c4ccccc34)c3ncc(C(C)CC)n3c2)c1 ; CCC(C)c1cnc2c(NCCc3c[nH]c4ccccc34)cc(-c3cncc(C4CC4)c3)cn12 ; COCCC(C)c1cnc2c(NCCc3c[nH]c4ccccc34)cc(-c3cncc(F)c3)cn12]

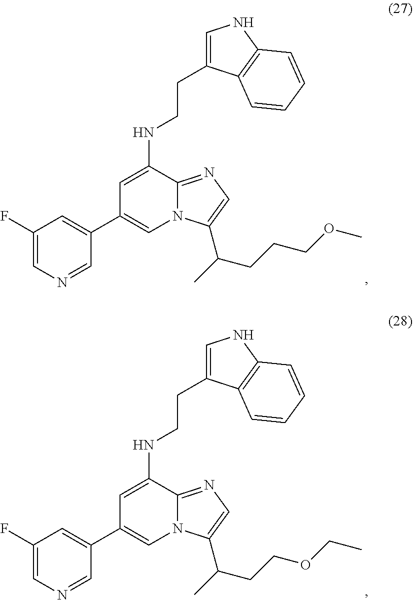 [SMILES: COCCCC(C)c1cnc2c(NCCc3c[nH]c4ccccc34)cc(-c3cncc(F)c3)cn12 ; CCOCCC(C)c1cnc2c(NCCc3c[nH]c4ccccc34)cc(-c3cncc(F)c3)cn12]

or a salt thereof.

In some embodiments, the aryl hydrocarbon receptor antagonist is compound (14), compound (15), compound (16), compound (17), compound (18), compound (19), compound (20), compound (21), compound (22), compound (23), compound (24), compound (26), compound (29), or compound (30)

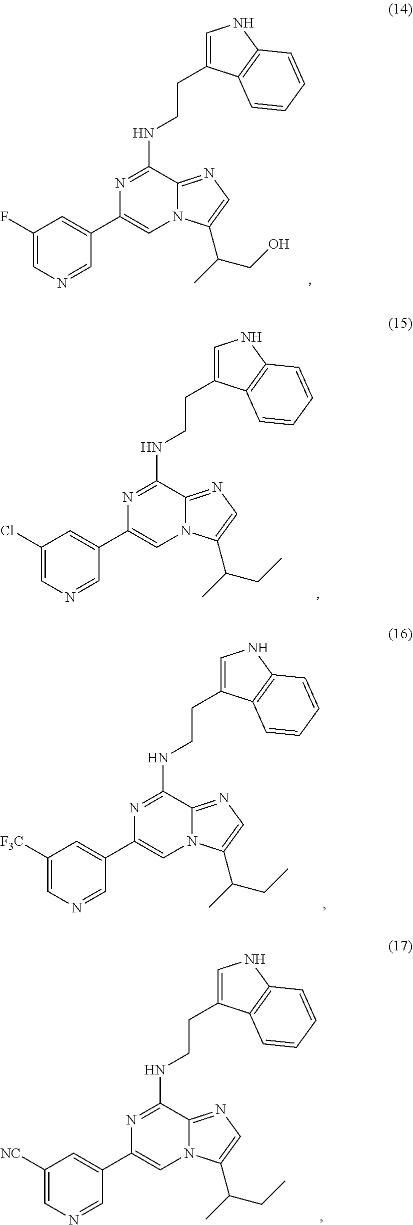 [SMILES: CC(CO)c1cnc2c(NCCc3c[nH]c4ccccc34)nc(-c3cncc(F)c3)cn12 ; CCC(C)c1cnc2c(NCCc3c[nH]c4ccccc34)nc(-c3cncc(Cl)c3)cn12 ; CCC(C)c1cnc2c(NCCc3c[nH]c4ccccc34)nc(-c3cncc(C(F)(F)F)c3)cn12 ; CCC(C)c1cnc2c(NCCc3c[nH]c4ccccc34)nc(-c3cncc(C#N)c3)cn12]

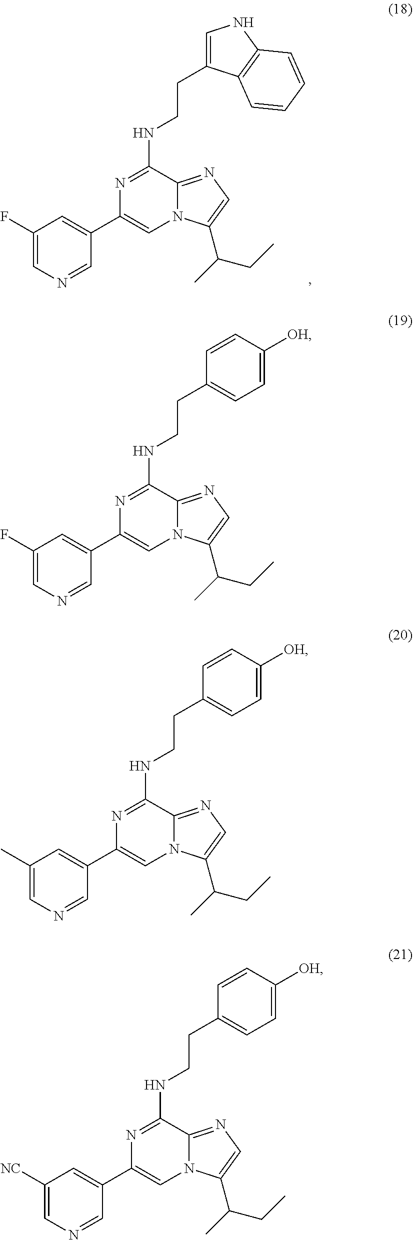 [SMILES: CCC(C)c1cnc2c(NCCc3c[nH]c4ccccc34)nc(-c3cncc(F)c3)cn12 ; CCC(C)c1cnc2c(NCCc3ccc(O)cc3)nc(-c3cncc(F)c3)cn12 ; CCC(C)c1cnc2c(NCCc3ccc(O)cc3)nc(-c3cncc(C)c3)cn12 ; CCC(C)c1cnc2c(NCCc3ccc(O)cc3)nc(-c3cncc(C#N)c3)cn12]

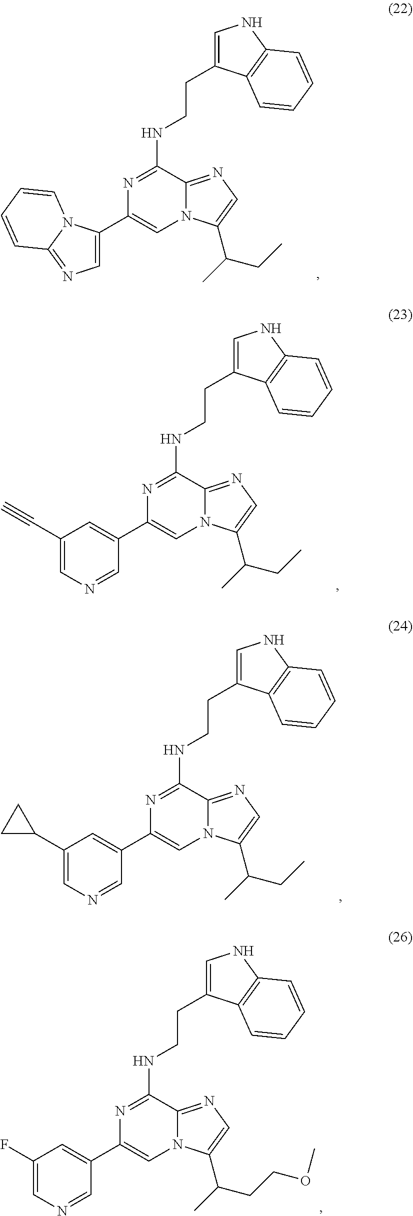 [SMILES: CCC(C)c1cnc2c(NCCc3c[nH]c4ccccc34)nc(-c3cnc4ccccn34)cn12 ; C#Cc1cncc(-c2cn3c(C(C)CC)cnc3c(NCCc3c[nH]c4ccccc34)n2)c1 ; CCC(C)c1cnc2c(NCCc3c[nH]c4ccccc34)nc(-c3cncc(C4CC4)c3)cn12 ; COCCC(C)c1cnc2c(NCCc3c[nH]c4ccccc34)nc(-c3cncc(F)c3)cn12]

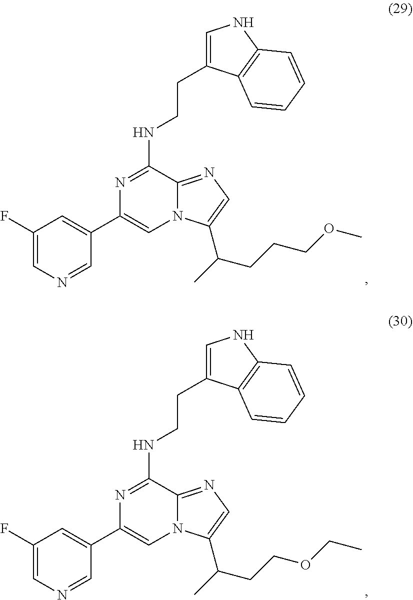 [SMILES: COCCCC(C)c1cnc2c(NCCc3c[nH]c4ccccc34)nc(-c3cncc(F)c3)cn12 ; CCOCCC(C)c1cnc2c(NCCc3c[nH]c4ccccc34)nc(-c3cncc(F)c3)cn12]

or a salt thereof.

### Populations of Hematopoietic Stem and Progenitor Cells

The methods described herein can give rise to a population of hematopoietic stem and/or progenitor cells useful for transplant to a donor. In various embodiments, the methods described herein give rise to a population of CD34⁺CD90⁺CD45RA⁻ at an increased amount as compared to a population of CD34⁺CD90⁺CD45RA⁻ mobilized by G-CSF.

In certain embodiments, the population comprises between about 5 to about 30 CD34⁺CD90⁺CD45RA⁻ cells/μL of peripheral blood, such as from about 5 to about 8 CD34⁺CD90⁺CD45RA⁻ cells/μL, about 5 to about 10 CD34⁺CD90⁺CD45RA⁻ cells/μL, about 5 to about 12 CD34⁺CD90⁺CD45RA⁻ cells/μL, about 5 to about 15 CD34⁺CD90⁺CD45RA⁻ cells/μL, about 5 to about 18 CD34⁺CD90⁺CD45RA⁻ cells/μL, about 5 to about 20 CD34⁺CD90⁺CD45RA⁻ cells/μL, about 5 to about 22 CD34⁺CD90⁺CD45RA⁻ cells/μL, about 5 to about 25 CD34⁺CD90⁺CD45RA⁻ cells/μL, about 5 to about 28 CD34⁺CD90⁺CD45RA⁻ cells/μL, about 8 to about 10 CD34⁺CD90⁺CD45RA⁻ cells/μL, about 8 to about 12 CD34⁺CD90⁺CD45RA⁻ cells/μL, about 8 to about 15 CD34⁺CD90⁺CD45RA⁻ cells/μL, or about 8 to about 18 CD34⁺CD90⁺CD45RA⁻ cells/μL of peripheral blood, about 8 to about 20 CD34⁺CD90⁺CD45RA⁻ cells/μL, about 8 to about 22 CD34⁺CD90⁺CD45RA⁻ cells/μL, about 8 to about 25 CD34⁺CD90⁺CD45RA⁻ cells/μL, about 8 to about 28 CD34⁺CD90⁺CD45RA⁻ cells/μL, about 8 to about 30 CD34⁺CD90⁺CD45RA⁻ cells/μL, about 10 to about 12 CD34⁺CD90⁺CD45RA⁻ cells/μL, about 10 to about 15 CD34⁺CD90⁺CD45RA⁻ cells/μL, about 10 to about 18 CD34⁺CD90⁺CD45RA⁻ cells/μL, about 10 to about 20 CD34⁺CD90⁺CD45RA⁻ cells/μL of peripheral blood, about 12 to about 15 CD34⁺CD90⁺CD45RA⁻ cells/μL, about 10 to about 18 CD34⁺CD90⁺CD45RA⁻ cells/μL, about 10 to about 20 CD34⁺CD90⁺CD45RA⁻ cells/μL of peripheral blood, about 10 to about 22 CD34⁺CD90⁺CD45RA⁻ cells/μL, about 10 to about 25 CD34⁺CD90⁺CD45RA⁻ cells/μL, about 10 to about 28 CD34⁺CD90⁺CD45RA⁻ cells/μL, about 10 to about 30 CD34⁺CD90⁺CD45RA⁻ cells/μL of peripheral blood, about 12 to about 15 CD34⁺CD90⁺CD45RA⁻ cells/μL, about 12 to about 18 CD34⁺CD90⁺CD45RA⁻ cells/μL, about 12 to about 15 CD34⁺CD90⁺CD45RA⁻ cells/μL, about 12 to about 18 CD34⁺CD90⁺CD45RA⁻ cells/μL, about 12 to about 20 CD34⁺CD90⁺CD45RA⁻ cells/μL, about 12 to about 22 CD34⁺CD90⁺CD45RA⁻ cells/μL, about 12 to about 25 CD34⁺CD90⁺CD45RA⁻ cells/μL, about 12 to about 28 CD34⁺CD90⁺CD45RA⁻ cells/μL, about 12 to about 30 CD34⁺CD90⁺CD45RA⁻ cells/μL, about 15 to about 18 CD34⁺CD90⁺CD45RA⁻ cells/μL, about 15 to about 20 CD34⁺CD90⁺CD45RA⁻ cells/μL, about 15 to about 22 CD34⁺CD90⁺CD45RA⁻ cells/μL, or about 15 to about 25 CD34⁺CD90⁺CD45RA⁻ cells/μL, about 15 to about 28 CD34⁺CD90⁺CD45RA⁻ cells/μL, about 15 to about 30 CD34⁺CD90⁺CD45RA⁻ cells/μL, about 18 to about 20 CD34⁺CD90⁺CD45RA⁻ cells/μL, about 18 to about 22 CD34⁺CD90⁺CD45RA⁻ cells/μL, or about 18 to about 25 CD34⁺CD90⁺CD45RA⁻ cells/μL, about 18 to about 28 CD34⁺CD90⁺CD45RA⁻ cells/μL, about 18 to about 30 CD34⁺CD90⁺CD45RA⁻ cells/μL, about 20 to about 22 CD34⁺CD90⁺CD45RA⁻ cells/μL, or about 20 to about 25 CD34⁺CD90⁺CD45RA⁻ cells/μL, about 20 to about 28 CD34⁺CD90⁺CD45RA⁻ cells/μL, about 20 to about 30 CD34⁺CD90⁺CD45RA⁻ cells/μL, about 22 to about 25 CD34⁺CD90⁺CD45RA⁻ cells/μL, about 22 to about 28 CD34⁺CD90⁺CD45RA⁻ cells/μL, about 22 to about 30 CD34⁺CD90⁺CD45RA⁻ cells/μL, about 25 to about 28 CD34⁺CD90⁺CD45RA⁻ cells/μL, about 25 to about 30 CD34⁺CD90⁺CD45RA⁻ cells/μL, or about 28 to about 30 CD34⁺CD90⁺CD45RA⁻ cells/μL.

In certain embodiments, wherein the donor has been diagnosed with multiple myeloma, the methods described herein can give rise to a population of hematopoietic stem and/or progenitor cells negative for minimal residual disease (MRD). MRD can be measured, for example, by flow cytometry or next-generation sequencing-based assays.

In certain embodiments, the population of hemopoietic stem or progenitor cells further comprises DMSO or citrate, and optionally has been frozen, e.g., using liquid nitrogen.

### Apheresis

In certain embodiments, the disclosure relates to a method of performing apheresis on the peripheral blood of a donor to produce an apheresis product, wherein the donor has been administered a CXCR2 agonist according to the methods described herein. In certain embodiments, about 10 L to about 30 L of peripheral blood is processed. In certain embodiments, apheresis occurs over a period of time of from about 3 hours to about 5 hours. In certain embodiments, the apheresis product has a volume of about 20 to about 400 mL. In certain embodiments, CD34⁺ cells are present in the apheresis product in an amount of from about 100×10⁶ cells to 600×10⁶ cells. For example, CD34⁺ cells can be present in the apheresis product in an amount of from about 100×10⁶ cells to 600×10⁶ cells. In some embodiments, CD34⁺ cells are present in the apheresis product in an amount of from about 200×10⁶ cells to 550×10⁶ cells, from about 300×10⁶ cells to 500×10⁶ cells, from about 350×10⁶ cells to 450×10⁶ cells, or from about 375×10⁶ cells to 400×10⁶ cells. In some embodiments, CD34⁺ cells are present in the apheresis product in an amount of from about 275×10⁶ cells to 375×10⁶ cells, from about 300×10⁶ cells to 350×10⁶ cells, or from about 310×10⁶ cells to 330×10⁶ cells.

In some embodiments, the apheresis product described herein possesses advantageous properties as compared to an apheresis product obtained from a donor mobilized using G-CSF. For example, in some embodiments, the apheresis product described herein may prevent, reduce the risk of developing, or reduce the severity of graft versus host disease (GVHD) in a patient in need thereof as compared to an apheresis product obtained from a donor administered G-CSF, where the apheresis product obtained from the donor administered G-CSF comprises hematopoietic stem cells that were mobilized into the peripheral blood of the donor following administration to the donor of a therapeutically effective amount of G-CSF. In certain embodiments, use of an apheresis product described herein in a patient undergoing a hematopoietic stem cell transplant may provide an increased engraftment rate for the hematopoietic stem cell transplant in the patient as compared to an apheresis product comprising hematopoietic stem cells that were mobilized into the peripheral blood of a donor administered a therapeutically effective amount of G-CSF.

In certain embodiments, CD34+ cells are present in the apheresis product in an amount of from about 1×10⁶ cells/kg body weight of the recipient to about 6×10⁶ cells/kg body weight of the recipient. As used herein, unless indicated otherwise, units expressed as cells/kg refers to cells/kg body weight of a recipient. In certain embodiments, the recipient is about 70 kg. In certain embodiments, the recipient is about 5 kg to about 150 kg. In certain embodiments, CD34+ cells are present in an amount of from about 1×10⁶ cells/kg to about 6×10⁶ cells/kg. In certain embodiments, CD34⁺ cells are present in an amount of from about 2×10⁶ cells/kg to about 5.75×10⁶ cells/kg, from about 3×10⁶ cells/kg to about 5.5×10⁶ cells/kg, from about 3.5×10⁶ cells/kg to about 5.25×10⁶ cells/kg, from about 4.0×10⁶ cells/kg to about 5.0×10⁶ cells/kg, from about 4.25×10⁶ cells/kg to about 4.75×10⁶ cells/kg, or from about 4.4×10⁶ cells/kg to about 4.6×10⁶ cells/kg. In certain embodiments, the CD34+ cells are viable CD34+ cells. In particular embodiments, CD34+ cells are present in an amount of at least about 2×10⁶ cells/kg. In particular embodiments, CD34+ cells are present in an amount of at least about 4×10⁶ cells/kg. In particular embodiments, CD34+ cells are present in an amount of at least about 6×10⁶ cells/kg.

In certain embodiments, CD34+CD90+CD45RA− cells are present in the apheresis product in an amount of from about 0.1×10⁶ cells/kg to about 5×10⁶ cells/kg. In certain embodiments, CD34+CD90+ cells are present in the apheresis product in an amount of from about 0.1×10⁶ cells/kg to about 5×10⁶ cells/kg.

In certain embodiments, the disclosure relates to an apheresis product isolated from a donor comprising CD34+CD90+CD45RA− cells in an amount of from about 0.1×10⁶ cells/kg body weight of the recipient to about 5×10⁶ cells/kg body weight or at a frequency of about 15 to about 75% of CD34+ cells present in the apheresis product. For example, the apheresis product isolated from a donor can comprise CD34+CD90+CD45RA− cells in an amount from about 0.2×10⁶ cells/kg to about 4×10⁶ cells/kg, from about 0.5×10⁶ cells/kg to about 3×10⁶ cells/kg, from about 1.0×10⁶ cells/kg to about 3×10⁶ cells/kg, from about 1.2×10⁶ cells/kg to about 2×10⁶ cells/kg, from about 1.4×10⁶ cells/kg to about 1.8×10⁶ cells/kg, or from about 1.5×10⁶ cells/kg to about 1.7×10⁶ cells/kg. In some embodiments, the apheresis product isolated from a donor comprises CD34+CD90+CD45RA− cells at a frequency of about 15 to about 50% of CD34+ cells present in the apheresis product. In some embodiments, the apheresis product isolated from a donor comprises CD34+CD90+CD45RA− cells at a frequency of about 20 to about 60% of CD34+ cells present in the apheresis product. In some embodiments, the apheresis product isolated from a donor comprises CD34+CD90+CD45RA− cells at a frequency of about 25 to about 55%, 25 to about 50%, 25 to about 40%, 30 to about 35%, about 31 to about 34%, or about 32 to about 33% of CD34+ cells present in the apheresis product. In particular embodiments, the apheresis product isolated from a donor comprises CD34+CD90+CD45RA− cells at a frequency of about 25 to about 55% of CD34+ cells present in the apheresis product. In particular embodiments, the apheresis product isolated from a donor comprises CD34+CD90+CD45RA− cells at a frequency of about 30 to about 40% of CD34+ cells present in the apheresis product. In particular embodiments, the apheresis product isolated from a donor comprises CD34+CD90+CD45RA− cells at a frequency of about 30 to about 35% of CD34+ cells present in the apheresis product. In particular embodiments, the apheresis product isolated from a donor comprises CD34+CD90+CD45RA− cells at a frequency of about 31% of CD34+ cells present in the apheresis product. In certain embodiments, the CD34+CD90+CD45RA− cells are viable CD34+CD90+CD45RA− cells.

In certain embodiments, the disclosure relates to an apheresis product isolated from a donor comprising CD34+ cells in an amount of from about 1.0×10⁶ cells/kg body weight of the recipient to about 8.0×10⁶ cells/kg body weight. In certain embodiments, the disclosure relates to an apheresis product isolated from a donor comprising CD34+ cells in an amount of from about 1.5×10⁶ cells/kg body weight of the recipient to about 7.0×10⁶ cells/kg body weight. For example, the apheresis product can comprise CD34+ cells in an amount from about 1.5×10⁶ cells/kg to about 7.0×10⁶ cells/kg, from about 2.0×10⁶ cells/kg to about 6.0×10⁶ cells/kg, from about 2.5×10⁶ cells/kg to about 5.0×10⁶ cells/kg, from about 3.0×10⁶ cells/kg to about 4.5×10⁶ cells/kg, from about 3.5×10⁶ cells/kg to about 4.0×10⁶ cells/kg, or from about 3.6×10⁶ cells/kg to about 3.8×10⁶ cells/kg.

In certain embodiments, the disclosure relates to an apheresis product isolated from a donor comprising CD3+ cells in an amount of from about 3.0×10⁸ cells/kg body weight of the recipient to about 6.5×10⁸ cells/kg body weight or at a frequency of about 35 to about 55% of CD45+ cells present in the apheresis product. In certain embodiments, the disclosure relates to an apheresis product isolated from a donor comprising CD3+ cells in an amount of from about 3.3×10⁸ cells/kg body weight of the recipient to about 6.2×10⁸ cells/kg body weight or at a frequency of about 31.7 to about 51.1% of CD45+ cells present in the apheresis product. For example, the apheresis product can comprise CD3+ cells in an amount from about 3.5×10⁸ cells/kg to about 6.0×10⁸ cells/kg, from about 3.75×10⁸ cells/kg to about 5.5×10⁸ cells/kg, from about 4.0×10⁸ cells/kg to about 5.0×10⁸ cells/kg, from about 4.25×10⁸ cells/kg to about 4.75×10⁸ cells/kg, from about 4.35×10⁸ cells/kg to about 4.65×10⁸ cells/kg, or from about 4.45×10⁸ cells/kg to about 4.55×10⁸ cells/kg. In some embodiments, the apheresis product comprises CD3+ cells at a frequency of about 32 to about 51% of CD45+ cells present in the apheresis product. In some embodiments, the apheresis product comprises CD3+ cells at a frequency of about 34 to about 48% of CD45+ cells present in the apheresis product. In some embodiments, the apheresis product comprises CD3+ cells at a frequency of about 36 to about 46%, 38 to about 44%, about 39 to about 42%, or about 40 to about 41% of CD45+ cells present in the apheresis product.

In certain embodiments, the disclosure relates to an apheresis product isolated from a donor comprising CD4+ cells in an amount of from about 3.0×10⁸ cells/kg body weight of the recipient to about 5.0×10⁸ cells/kg body weight or at a frequency of about 25 to about 50% of CD45+ cells present in the apheresis product. In certain embodiments, the disclosure relates to an apheresis product isolated from a donor comprising CD4+ cells in an amount of from about 3.0×10⁸ cells/kg body weight of the recipient to about 5.0×10⁸ cells/kg body weight or at a frequency of about 27.2 to about 48.1% of CD45+ cells present in the apheresis product. For example, the apheresis product can comprise CD4+ cells in an amount from about 3.2×10⁸ cells/kg to about 4.8×10⁸ cells/kg, from about 3.3×10⁸ cells/kg to about 4.6×10⁸ cells/kg, from about 3.4×10⁸ cells/kg to about 4.4×10⁸ cells/kg, from about 3.5×10⁸ cells/kg to about 4.2×10⁸ cells/kg, from about 3.6×10⁸ cells/kg to about 4.0×10⁸ cells/kg, or from about 3.7×10⁸ cells/kg to about 3.8×10⁸ cells/kg. In some embodiments, the apheresis product comprises CD4+ cells at a frequency of about 28 to about 48% of CD45+ cells present in the apheresis product. In some embodiments, the apheresis product comprises CD4+ cells at a frequency of about 30 to about 45% of CD45+ cells present in the apheresis product. In some embodiments, the apheresis product comprises CD4+ cells at a frequency of about 31 to about 40%, 32 to about 36%, about 32 to about 35%, or about 33 to about 34% of CD45+ cells present in the apheresis product.

In certain embodiments, the disclosure relates to an apheresis product isolated from a donor comprising CD8+ cells in an amount of from about 0.0×10⁸ cells/kg body weight of the recipient to about 1.0×10⁸ cells/kg body weight or at a frequency of about 0.5 to about 5% of CD45+ cells present in the apheresis product. In certain embodiments, the disclosure relates to an apheresis product isolated from a donor comprising CD8+ cells in an amount of from about 0.0×10⁸ cells/kg body weight of the recipient to about 0.6×10⁸ cells/kg body weight or at a frequency of about 0.5 to about 4.8% of CD45+ cells present in the apheresis product. For example, the apheresis product can comprise CD8+ cells in an amount from about 0.1×10⁸ cells/kg to about 0.55×10⁸ cells/kg, from about 0.12×10⁸ cells/kg to about 0.50×10⁸ cells/kg, from about 0.14×10⁸ cells/kg to about 0.40×10⁸ cells/kg, from about 0.16×10⁸ cells/kg to about 0.30×10⁸ cells/kg, from about 0.18×10⁸ cells/kg to about 0.25×10⁸ cells/kg, or from about 0.20×10⁸ cells/kg to about 0.22×10⁸ cells/kg. In some embodiments, the apheresis product comprises CD8+ cells at a frequency of about 0.5 to about 4.8% of CD45+ cells present in the apheresis product. In some embodiments, the apheresis product comprises CD8+ cells at a frequency of about 0.6 to about 4.0% of CD45+ cells present in the apheresis product. In some embodiments, the apheresis product comprises CD8+ cells at a frequency of about 0.8 to about 3.5%, 1.2 to about 3.0%, about 1.4 to about 2.5%, or about 1.6 to about 2.0% of CD45+ cells present in the apheresis product.

In certain embodiments, the disclosure relates to an apheresis product isolated from a donor comprising CD19+ cells in an amount of from about 1.0×10⁸ cells/kg body weight of the recipient to about 2×10⁸ cells/kg body weight or at a frequency of about 10 to about 20% of CD45+ cells present in the apheresis product. In certain embodiments, the disclosure relates to an apheresis product isolated from a donor comprising CD19+ cells in an amount of from about 1.1×10⁸ cells/kg body weight of the recipient to about 1.9×10⁸ cells/kg body weight or at a frequency of about 12.3 to about 19.7% of CD45+ cells present in the apheresis product. For example, the apheresis product can comprise CD19+ cells in an amount from about 1.2×10⁸ cells/kg to about 1.9×10⁸ cells/kg, from about 1.3×10⁸ cells/kg to about 1.9×10⁸ cells/kg, from about 1.4×10⁸ cells/kg to about 1.9×10⁸ cells/kg, from about 1.5×10⁸ cells/kg to about 1.9×10⁸ cells/kg, from about 1.6×10⁸ cells/kg to about 1.9×10⁸ cells/kg, from about 1.7×10⁸ cells/kg to about 1.9×10⁸ cells/kg, or from about 1.8×10⁸ cells/kg to about 1.9×10⁸ cells/kg. In some embodiments, the apheresis product comprises CD19+ cells at a frequency of about 0.2 to about 1.0% of CD45+ cells present in the apheresis product. In some embodiments, the apheresis product comprises CD19+ cells at a frequency of about 12.5 to about 19.5% of CD45+ cells present in the apheresis product. In some embodiments, the apheresis product comprises CD19+ cells at a frequency of about 13.0 to about 18%, 13.5 to about 17.0%, about 14.0 to about 16.0% of CD45+ cells, or about 14.5 to about 15.0% of CD45+ cells present in the apheresis product.

In certain embodiments, the disclosure relates to an apheresis product isolated from a donor comprising CD56+ cells in an amount of from about 0.2×10⁸ cells/kg body weight of the recipient to about 1.0×10⁸ cells/kg body weight or at a frequency of about 2 to about 9% of CD45+ cells present in the apheresis product. In certain embodiments, the disclosure relates to an apheresis product isolated from a donor comprising CD56+ cells in an amount of from about 0.2×10⁸ cells/kg body weight of the recipient to about 1.0×10⁸ cells/kg body weight or at a frequency of about 2.1 to about 8.3% of CD45+ cells present in the apheresis product. For example, the apheresis product can comprise CD56+ cells in an amount from about 0.3×10⁸ cells/kg to about 0.9×10⁸ cells/kg, from about 0.35×10⁸ cells/kg to about 0.8×10⁸ cells/kg, from about 0.40×10⁸ cells/kg to about 0.7×10⁸ cells/kg, from about 0.45×10⁸ cells/kg to about 0.6×10⁸ cells/kg, or from about 0.475×10⁸ cells/kg to about 0.55×10⁸ cells/kg. In some embodiments, the apheresis product comprises CD56+ cells at a frequency of about 2.5 to about 8.0% of CD45+ cells present in the apheresis product. In some embodiments, the apheresis product comprises CD56+ cells at a frequency of about 3.0 to about 7.0% of CD45+ cells present in the apheresis product. In some embodiments, the apheresis product comprises CD56+ cells at a frequency of about 3.5 to about 6.5%, 4.0 to about 6.0%, or about 4.5 to about 5.5% of CD45+ cells present in the apheresis product.

In certain embodiments, the disclosure relates to an apheresis product isolated from a donor comprising Treg cells at a frequency of about 0.5 to about 6% of CD4+ T cells. In certain embodiments, the disclosure relates to an apheresis product isolated from a donor comprising Treg cells at a frequency of about 0.7 to about 5.5% of CD4+ T cells. In some embodiments, the apheresis product comprises Treg at a frequency of about 1.0 to about 5.0% of CD4+ T cells present in the apheresis product. In some embodiments, the apheresis product comprises Tregs at a frequency of about 1.5 to about 4.5%, 1.75 to about 4.0%, about 2.0 to about 3.5%, about 2.25 to about 3.0%, or about 2.5 to about 2.75% of CD4+ T cells present in the apheresis product.

In certain embodiments, the disclosure relates to an apheresis product isolated from a donor comprising NKT cells at a frequency of about 0.5 to about 3% of CD3+ T cells. In certain embodiments, the disclosure relates to an apheresis product isolated from a donor comprising NKT cells at a frequency of about 0.6 to about 2.7% of CD3+ T cells. In some embodiments, the apheresis product comprises NKT cells at a frequency of about 0.7 to about 2.5% of CD3+ T cells present in the apheresis product. In some embodiments, the apheresis product comprises NKT cells at a frequency of about 0.8 to about 2.4%, 0.9 to about 2.3%, about 1.0 to about 2.2%, about 1.1 to about 2.1%, about 1.2 to about 2.0%, about 1.3 to about 1.9%, about 1.4 to about 1.8%, or about 1.5 to about 1.7% of CD3+ T cells present in the apheresis product.

In certain embodiments, the disclosure relates to an apheresis product isolated from a donor comprising iNKT cells at a frequency of about 0.00 to about 0.1% of CD3+ T cells. In certain embodiments, the disclosure relates to an apheresis product isolated from a donor comprising iNKT cells at a frequency of about 0.00 to about 0.03% of CD3+ T cells. In some embodiments, the apheresis product comprises iNKT cells at a frequency of about 0.001 to about 0.025%, 0.005 to about 0.020%, about 0.007 to about 0.015%, about 0.008 to about 0.012%, or about 0.009 to about 0.011% of CD3+ T cells present in the apheresis product.

In certain embodiments, the disclosure relates to an apheresis product isolated from a donor comprising CD14+ monocytes. CD14+ monocytes possess immunosuppressive effects such that when the apheresis product is administered, the CD14+ monocytes exert the immunosuppressive effects, thereby preventing or reducing the severity of GvHD. In various embodiments, CD14+ cells are present in the apheresis product in an amount of from about 1×10⁶ cells/kg body weight of the recipient to 1000×10⁶ cells/kg body weight. For example, CD34+ cells can be present in the apheresis product in an amount of from about 1×10⁶ cells/kg body weight to 1000×10⁶ cells/kg body weight. In some embodiments, CD34+ cells are present in the apheresis product in an amount of from about 10×10⁶ cells/kg body weight to 900×10⁶ cells/kg body weight, from about 100×10⁶ cells/kg body weight to 800×10⁶ cells/kg body weight, from about 250×10⁶ cells/kg body weight to 750×10⁶ cells/kg body weight, from about 375×10⁶ cells/kg body weight to 600×10⁶ cells/kg body weight, or from about 425×10⁶ cells/kg body weight to 550×10⁶ cells/kg body weight.

In certain embodiments, the concentration of white blood cells is higher in the apheresis product than in the peripheral blood of the donor. In certain embodiments, the apheresis product further comprises an anticoagulant. In certain embodiments, citrate in an amount above physiological levels. In certain embodiments, the anticoagulant is heparin. In certain embodiments, the volume of the product is from about 20 to about 400 mL.

In certain embodiments, the disclosure relates to a method of treating a stem cell disorder, the method comprising administering the apheresis product or population of hemopoietic stem or progenitor cells described herein. It has been surprisingly discovered that the composition of apheresis products obtained using the methods described herein are unexpectedly potent. In certain embodiments, apheresis products obtained from a donor in which apheresis yields are lower than 2×10⁶ CD34+ cells/kg body weight (e.g., five times lower, 10 times lower) are still efficacious.

In certain embodiments, wherein the donor has been diagnosed with multiple myeloma, the apheresis product is negative for minimal residual disease (MRD). MRD can be measured, for example, by flow cytometry or next-generation sequencing-based assays.

In various embodiments, apheresis is performed on a donor on multiple days such that multiple apheresis products are generated. In various embodiments, apheresis is performed on a donor on at least two days, such that at least two apheresis products are generated. In various embodiments, apheresis is performed on a donor on at least two consecutive days, such that an apheresis product is generated for each performed apheresis. In some embodiments, the multiple apheresis products may be combined and administered to a recipient as a single apheresis product.

### Kinetics of CXCR2 Agonist and CXCR4 Antagonist Dosing

For cases in which the donor is administered both a CXCR4 antagonist and a CXCR2 agonist, the two agents may be administered to the donor substantially simultaneously (e.g., at the same time or one immediately after the other). In some embodiments, the CXCR4 antagonist and the CXCR2 agonist may be co-formulated with one another and administered in the same pharmaceutical composition. Alternatively, the CXCR4 antagonist and the CXCR2 agonist may be formulated in distinct pharmaceutical compositions and administered separately but substantially simultaneously to the donor.

In some embodiments, the CXCR2 agonist is administered to the donor after administration of the CXCR4 antagonist. In some embodiments, the CXCR2 agonist is administered to the donor within about 12 hours (e.g., within about 10, 8, 6, 4, 2, or 1 hour) of administration of the CXCR4 antagonist. In some embodiments, the CXCR2 agonist is administered to the donor from about 30 minutes to about 180 minutes after administration of the CXCR4 antagonist, such as from about 40 minutes to about 160 minutes, about 50 minutes to about 150 minutes, about 60 minutes to about 140 minutes, about 70 minutes to about 130 minutes, about 60 minutes to about 120 minutes, about 70 minutes to about 110 minutes, or about 80 minutes to about 100 minutes (e.g., about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes, or about 180 minutes after administration of the CXCR4 antagonist). In some embodiments, the CXCR2 agonist is administered about 2 hours after the CXCR4 antagonist.

In certain embodiments, peripheral blood containing a population of hematopoietic stem or progenitor cells is isolated from the donor from about 10 minutes to about 60 minutes following completion of the administration of the CXCR4 antagonist and the CXCR2 agonist (e.g., about 10 minutes to about 1.9 hours, about 20 minutes to about 1.8 hours, about 25 minutes to about 1.7 hours, about 30 minutes to about 1.6 hours, about 40 minutes to about 1.5 hours (e.g., about 10 minutes, about 15 minutes, about 20 minutes, about 25 minutes, about 30 minutes, about 35 minutes, about 40 minutes, about 45 minutes, about 50 minutes, about 55 minutes, about 60 minutes, or about 120 minutes following completion of the administration of the CXCR4 antagonist and the CXCR2 agonist). In certain embodiments, peripheral blood containing a population of hematopoietic stem or progenitor cells is isolated from the donor from about 10 minutes to about 20 minutes following completion of the administration of the CXCR4 antagonist and the CXCR2 agonist (e.g., about 10 minutes, about 11 minutes, about 12 minutes, about 13 minutes, about 14 minutes, about 15 minutes, about 16 minutes, about 17 minutes, about 18 minutes, about 19 minutes, or about 20 minutes following completion of the administration of the CXCR4 antagonist and the CXCR2 agonist). In some embodiments, isolation of the population of hematopoietic stem or progenitor cells commences about 15 minutes following completion of the administration of the CXCR4 antagonist and the CXCR2 agonist.

In certain embodiments, peripheral blood containing a population of hematopoietic stem or progenitor cells is isolated from the donor between about 15 minutes to about 10 hours after administration of the CXCR2 agonist and/or the CXCR4 antagonist, e.g., between about 15 minutes to about 5 hours, between about 15 minutes to about 2 hours, between about 15 minutes and about 1 hour, between about 15 minutes and about 30 minutes, between about 30 minutes and about 10 hours, between about 30 minutes and about 5 hours, between about 30 minutes and about 2 hours, between about 30 minutes and about 1 hour, between about 1 hour and about 10 hours, between about 1 hour and about 5 hours, between about 1 hour and about 2 hours, between about 2 hours to about 3 hours, between about 2 hours to about 4 hours, between about 2 hours to about 5 hours, between about 2 hours to about 6 hours, between about 2 hours to about 7 hours, between about 2 hours about 8 hours, between about 2 hours to about 9 hours, between about 3 hours to about 4 hours, between about 3 hours to about 5 hours, between about 3 hours to about 6 hours, between about 3 hours to about 7 hours, between about 3 hours about 8 hours, between about 3 hours to about 9 hours, between about 3 hours to about 10 hours, between about 4 hours to about 5 hours, between about 4 hours to about 6 hours, between about 4 hours to about 7 hours, between about 4 hours about 8 hours, between about 4 hours to about 9 hours, between about 4 hours to about 10 hours, between about 5 hours to about 6 hours, between about 5 hours to about 7 hours, between about 5 hours about 8 hours, between about 5 hours to about 9 hours, between about 5 hours to about 10 hours, between about 6 hours to about 7 hours, between about 6 hours about 8 hours, between about 6 hours to about 9 hours, between about 6 hours to about 10 hours, between about 7 hours to 8 hours, between about 7 hours to about 9 hours, between about 7 hours to about 10 hours, between about 8 hours to about 9 hours, between about 8 hours to about 10 hours, or between about 9 hours to about 10 hours. In particular embodiments, peripheral blood containing a population of hematopoietic stem or progenitor cells is isolated from the donor between about 15 minutes and about 10 hours after administration of the CXCR2 agonist and/or the CXCR4 antagonist. In particular embodiments, peripheral blood containing a population of hematopoietic stem or progenitor cells is isolated from the donor between about 30 minutes and about 1 hour after administration of the CXCR2 agonist and/or the CXCR4 antagonist. In particular embodiments, peripheral blood containing a population of hematopoietic stem or progenitor cells is isolated from the donor at about 30 mintues after administration of the CXCR2 agonist and/or the CXCR4 antagonist.

In some embodiments, the population of hematopoietic stem or progenitor cells is isolated from the donor over a period of from about 15 minutes to about 3 hours, such as from about 20 minutes to about 4.5 hours, about 30 minutes to about 4 hours, about 40 minutes to about 3.5 hours, about 50 minutes to about 3 hours, or about 1 hour to about 2 hours (e.g., over a period of about 15 minutes, about 20 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 180 minutes, about 240 minutes, about 300 minutes, or about 360 minutes). In some embodiments, the population of hematopoietic stem and progenitor cells may be isolated from the donor over a period of from about 30 minutes to about 1 hour (e.g., over a period of about 30 minutes, about 35 minutes, about 40 minutes, about 45 minutes, about 50 minutes, about 55 minutes, or about 60 minutes).

In some embodiments, the hematopoietic stem or progenitor cells may be harvested by apheresis. In some embodiments, the hematopoietic stem or progenitor cells may be harvested by drawing peripheral blood from the donor (i.e., subject).

### Routes of Administration of CXCR2 Agonists and CXCR4 Antagonists

The CXCR4 antagonists and CXCR2 agonists described herein may be administered to a patient by a variety of routes, such as intravenously, subcutaneously, intramuscularly, or parenterally. The most suitable route for administration in any given case will depend on the particular agent administered, the patient, pharmaceutical formulation methods, administration methods (e.g., administration time and administration route), the patient's age, body weight, sex, severity of the diseases being treated, the patient's diet, and the patient's excretion rate.

### Pharmaceutical Compositions

The CXCR2 agonists and CXCR4 antagonists contemplated herein may each be formulated into a pharmaceutical composition for administration to a subject, such as a mammalian subject (e.g., a human subject). For instance, contemplated herein are pharmaceutical compositions comprising a CXCR2 agonist and/or a CXCR4 antagonist, in admixture with one or more suitable diluents, carriers, and/or excipients. Pharmaceutical compositions may include sterile aqueous suspensions. Conventional procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33), the disclosure of which is incorporated herein by reference in its entirety.

A pharmaceutical composition may be administered to a subject, such as a human subject, alone or in combination with pharmaceutically acceptable carriers, the proportion of which may be determined by the quantity of active pharmaceutical ingredient (i.e., CXCR2 agonist and/or a CXCR4 antagonist), chosen route of administration, and standard pharmaceutical practice.

Administration and Dosing of CXCR2 Agonists and/or CXCR4 Antagonists

Contemplated CXCR2 agonists and CXCR4 antagonists, may be administered to a subject, such as a mammalian subject (e.g., a human subject), by one or more routes of administration. For instance, contemplated CXCR2 agonists and CXCR4 antagonists may be administered to a subject by intravenous, intraperitoneal, intramuscular, intraarterial, or subcutaneous infusion, among others. In various embodiments, CXCR2 agonists and CXCR4 antagonists can be administered to a subject who was previously diagnosed with a stem cell disorder or a hematological malignancy. As an example, CXCR2 agonists and CXCR4 antagonists can be administered to a subject who was previously diagnosed with a hematological malignancy such as multiple myeloma.

Contemplated CXCR2 agonists can be administered in an amount of between about 0.001 mg/kg to about 0.1 mg/kg body weight of the subject, for example, between about 0.05 mg/kg and about 0.1 mg/kg, between about 0.05 mg/kg about 0.07 mg/kg, and between about 0.07 mg/kg and about 0.1 mg/kg.

Contemplated CXCR2 agonists can be administered in an amount of between about 0.001 mg/kg and less than about 0.05 mg/kg body weight of the subject, for example, between about 0.0015 mg/kg and less than about 0.05 mg/kg, between about 0.002 mg/kg and less than about 0.05 mg/kg, between about 0.025 mg/kg and less than about 0.05 mg/kg, between about 0.003 mg/kg and less than about 0.05 mg/kg, between about 0.0035 mg/kg and less than about 0.05 mg/kg, between about 0.004 mg/kg and less than about 0.05 mg/kg, between about 0.0045 mg/kg and less than about 0.05 mg/kg, between about 0.005 mg/kg and less than about 0.05 mg/kg, between about 0.0055 mg/kg and less than about 0.05 mg/kg, between about 0.006 mg/kg and less than about 0.05 mg/kg, between about 0.0065 mg/kg and less than about 0.05 mg/kg, between about 0.007 mg/kg and less than about 0.05 mg/kg, between about 0.075 mg/kg and less than about 0.05 mg/kg, between about 0.008 mg/kg and less than about 0.05 mg/kg, between about 0.0085 mg/kg and less than about 0.05 mg/kg, between about 0.009 mg/kg and less than about 0.05 mg/kg, between about 0.0095 mg/kg and less than about 0.05 mg/kg, between about 0.01 mg/kg and less than about 0.05 mg/kg, between about 0.015 mg/kg and less than about 0.05 mg/kg, between about 0.02 and less than about 0.05 mg/kg, between about 0.025 mg/kg and less than about 0.05 mg/kg; between about 0.03 mg/kg and less than about 0.05 mg/kg, between about 0.035 mg/kg and less than about 0.05 mg/kg, between about 0.04 mg/kg and less than about 0.05 mg/kg, and between about 0.045 mg/kg and less than about 0.05 mg/kg.

In certain embodiments, the CXCR2 agonists can be administered in an amount of between about 0.001 mg/kg and about 0.049 mg/kg, for example, between about 0.001 mg/kg and about 0.045 mg/kg, between about 0.001 mg/kg and about 0.04 mg/kg, between about 0.001 mg/kg and about 0.035 mg/kg, between about 0.001 mg/kg and about 0.03 mg/kg, between about 0.001 mg/kg and about 0.025 mg/kg, between about 0.001 mg/kg and about 0.02 mg/kg, between about 0.001 mg/kg and about 0.015 mg/kg, between about 0.001 mg/kg and about 0.01 mg/kg.

In certain embodiments, the CXCR2 agonists can be administered in an amount of between about 0.01 mg/kg and less than about 0.05 mg/kg, between about 0.01 mg/kg and about 0.049 mg/kg, between about 0.01 mg/kg and about 0.045 mg/kg, between about 0.01 mg/kg and about 0.04 mg/kg, between about 0.01 mg/kg and about 0.035 mg/kg, between about 0.01 mg/kg and about 0.03 mg/kg, between about 0.01 mg/kg and about 0.025 mg/kg, between about 0.01 mg/kg and about 0.02 mg/kg, and between about 0.01 mg/kg and about 0.015 mg/kg.

In certain embodiments, the CXCR2 agonists can be administered in an amount of between about 0.02 mg/kg and less than about 0.05 mg/kg, between about 0.02 mg/kg and about 0.049 mg/kg, between about 0.02 mg/kg and about 0.045 mg/kg, between about 0.02 mg/kg and about 0.04 mg/kg, between about 0.02 mg/kg and about 0.035 mg/kg, between about 0.02 mg/kg and about 0.03 mg/kg, and between about 0.02 mg/kg and about 0.025 mg/kg.

In certain embodiments, the CXCR2 agonist is administered at a dose of about 0.03 mg/kg.

In certain embodiments, the CXCR2 agonist is administered at a fixed dose of from about 1 mg to about 8 mg. For example, the CXCR2 agonist can be administered at a fixed dose of from about 1 mg to about 1.5 mg, about 1 mg to about 2 mg, about 1 mg to about 2.5 mg, about 1 mg to about 3 mg, about 1 mg to about 3.5 mg, about 1 mg to about 4 mg, about 1 mg to about 4.5 mg, about 1 mg to about 5 mg, about 1 mg to about 5.5 mg, about 1 mg to about 6 mg, about 1 mg to about 6.5 mg, about 1 mg to about 7 mg, about 1 mg to about 7.5 mg, about 1.5 mg to about 2 mg, about 1.5 mg to about 2.5 mg, about 1.5 mg to about 3 mg, about 1.5 mg to about 3.5 mg, about 1.5 mg to about 4 mg, about 1.5 mg to about 4.5 mg, about 1.5 mg to about 5 mg, about 1.5 mg to about 5.5 mg, about 1.5 mg to about 6 mg, about 1.5 mg to about 6.5 mg, about 1.5 mg to about 7 mg, about 1.5 mg to about 7.5 mg, about 1.5 mg to about 8 mg, about 2 mg to about 2.5 mg, about 2 mg to about 3 mg, about 2 mg to about 3.5 mg, about 2 mg to about 4 mg, about 2 mg to about 4.5 mg, about 2 mg to about 5 mg, about 2 mg to about 5.5 mg, about 2 mg to about 6 mg, about 2 mg to about 6.5 mg, about 2 mg to about 7 mg, about 2 mg to about 7.5 mg, about 2 mg to about 8 mg, about 2.5 mg to about 3 mg, about 2.5 mg to about 3.5 mg, about 2.5 mg to about 4 mg, about 2.5 mg to about 4.5 mg, about 2.5 mg to about 5 mg, about 2.5 mg to about 5.5 mg, about 2.5 mg to about 6 mg, about 2.5 mg to about 6.5 mg, about 2.5 mg to about 7 mg, about 2.5 mg to about 7.5 mg, about 2.5 mg to about 8 mg, about 3 mg to about 3.5 mg, about 3 mg to about 4 mg, about 3 mg to about 4.5 mg, about 3 mg to about 5 mg, about 3 mg to about 5.5 mg, about 3 mg to about 6 mg, about 3 mg to about 6.5 mg, about 3 mg to about 7 mg, about 3 mg to about 7.5 mg, about 3 mg to about 8 mg, about 3.5 mg to about 4 mg, about 3.5 mg to about 4.5 mg, about 3.5 mg to about 5 mg, about 3.5 mg to about 5.5 mg, about 3.5 mg to about 6 mg, about 3.5 mg to about 6.5 mg, about 3.5 mg to about 7 mg, about 3.5 mg to about 7.5 mg, about 3.5 mg to about 8 mg, about 4 mg to about 4.5 mg, about 4 mg to about 5 mg, about 4 mg to about 5.5 mg, about 4 mg to about 6 mg, about 4 mg to about 6.5 mg, about 4 mg to about 7 mg, about 4 mg to about 7.5 mg, about 4 mg to about 8 mg, about 4.5 mg to about 5 mg, about 4.5 mg to about 5.5 mg, about 4.5 mg to about 6 mg, about 4.5 mg to about 6.5 mg, about 4.5 mg to about 7 mg, about 4.5 mg to about 7.5 mg, about 4.5 mg to about 8 mg, about 5 mg to about 5.5 mg, about 5 mg to about 6 mg, about 5 mg to about 6.5 mg, about 5 mg to about 7 mg, about 5 mg to about 7.5 mg, about 5 mg to about 8 mg, about 5.5 mg to about 6 mg, about 5.5 mg to about 6.5 mg, about 5.5 mg to about 7 mg, about 5.5 mg to about 7.5 mg, about 5.5 mg to about 8 mg, about 6 mg to about 6.5 mg, about 6 mg to about 7 mg, about 6 mg to about 7.5 mg, about 6 mg to about 8 mg, about 6.5 mg to about 7 mg, about 6.5 mg to about 7.5 mg, about 6.5 mg to about 8 mg, about 7 mg to about 7.5 mg, about 7 mg to about 8 mg, about 7.5 mg to 8 mg. In certain embodiments, the CXCR2 agonist is administered at a fixed dose of about 1.3 mg, 2.5 mg or 5.5 mg.

In certain embodiments, the CXCR2 agonists can be administered in an amount of about 0.001 mg/kg per day, about 0.0015 mg/kg per day, about 0.002 mg/kg per day, about 0.0025 mg/kg per day, about 0.003 mg/kg per day, about 0.0035 mg/kg per day, about 0.004 mg/kg per day, about 0.0045 mg/kg per day, about 0.005 mg/kg per day, about 0.0055 mg/kg per day, about 0.006 mg/kg per day, about 0.0065 mg/kg per day, about 0.007 mg/kg per day, about 0.0075 mg/kg per day, about 0.008 mg/kg per day, about 0.0085 mg/kg per day, about 0.009 mg/kg per day, about 0.0095 mg/kg per day, about 0.01 mg/kg per day, about 0.015 mg/kg per day, about 0.02 mg/kg per day, about 0.025 mg/kg per day, about 0.03 mg/kg per day, about 0.035 mg/kg per day, about 0.04 mg/kg per day, about 0.045 mg/kg per day, about 0.049 mg/kg per day, or less than about 0.05 mg/kg per day. In certain embodiments, the CXCR2 agonist is administered at a fixed dose of about 1.3 mg per day, 2.5 mg per day, or 5.5 mg per day.

In certain embodiments, the CXCR2 agonist is administered at a fixed dose of from about 1 mg to about 8 mg per day. For example, the CXCR2 agonist can be administered at a fixed dose of from about 1 mg per day, about 1.5 mg per day, about 2 mg per day, about 2.5 mg per day, about 3.5 mg per day, about 4 mg per day, about 5 mg per day, about 5.5 mg per day, about 6 mg per day, about 6.5 mg per day, about 7 mg per day, about 7.5 mg per day, or about 8 mg per day.

It was surprisingly discovered that administration of a CXCR2 agonist over a period of time that is less than about 20 minutes resulted in less acute and transient bone pain than when a CXCR2 agonist is administered over a longer period of time. It has also been surprisingly discovered that administration of a CXCR2 agonist over a period of time that is less than about 20 minutes resulted in faster resolution of acute and transient bone pain than when a CXCR2 agonist is administered over a longer period of time. Accordingly, in various embodiments, the CXCR2 agonist is administered over a period of time of from about 1 minute to about 20 minutes. In various embodiments, the CXCR2 agonist is administered over a period of time between about 1 minute to about 10 minutes, from about 1 minute to about 8 minutes, from about 1 minute to about 5 minutes, from about 1 minute to about 3 minutes, from about 2 minutes to about 20 minutes, from about 2 minutes to about 10 minutes, from about 2 minutes to about 8 minutes, from about 2 minutes to about 5 minutes, from about 2 minutes to about 3 minutes, from about 3 minutes to about 20 minutes, from about 3 minutes to about 10 minutes, from about 3 minutes to about 8 minutes, from about 3 minutes to about 5 minutes, from about 3 minutes to about 4 minutes, from about 4 minutes to about 20 minutes, from about 4 minutes to about 10 minutes, from about 4 minutes to about 8 minutes, from about 4 minutes to about 5 minutes, from about 5 minutes to about 20 minutes, from about 5 minutes to about 10 minutes, from about 5 minutes to about 8 minutes, from about 5 minutes to about 6 minutes, from about 6 minutes to about 10 minutes, from about 6 minutes to about 8 minutes, from about 6 minutes to about 7 minutes, from about 7 minutes to about 20 minutes, from about 7 minutes to about 10 minutes, from about 7 minutes to about 8 minutes, from about 8 minutes to about 20 minutes, from about 8 minutes to about 10 minutes, from about 8 minutes to about 9 minutes, from about 9 minutes to about 20 minutes, from about 9 minutes to about 10 minutes, or from about 10 minutes to about 20 minutes. In particular embodiments, the CXCR2 agonist is administered over a period of time of less than about 20 minutes. In particular embodiments, the CXCR2 agonist is administered over a period of time of less than about 15 minutes. In particular embodiments, the CXCR2 agonist is administered over a period of time of less than about 10 minutes. In particular embodiments, the CXCR2 agonist is administered over a period of time between about 3 minutes to about 10 minutes.

In some embodiments, the CXCR4 antagonist is plerixafor or a pharmaceutically acceptable salt thereof. In some embodiments, the CXCR4 antagonist (e.g., plerixafor or a pharmaceutically acceptable salt thereof) is administered subcutaneously to the donor. In some embodiments, the CXCR4 antagonist (e.g., plerixafor or a pharmaceutically acceptable salt thereof) is administered to the donor at a dose of from about 50 μg/kg to about 500 μg/kg body weight of the donor, such as a dose of about 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 105 μg/kg, 110 μg/kg, 115 μg/kg, 120 μg/kg, 125 μg/kg, 130 μg/kg, 135 μg/kg, 140 μg/kg, 145 μg/kg, 150 μg/kg, 155 μg/kg, 160 μg/kg, 165 μg/kg, 170 μg/kg, 175 μg/kg, 180 μg/kg, 185 μg/kg, 190 μg/kg, 195 μg/kg, 200 μg/kg, 205 μg/kg, 210 μg/kg, 215 μg/kg, 220 μg/kg, 225 μg/kg, 230 μg/kg, 235 μg/kg, 240 μg/kg, 245 μg/kg, 250 μg/kg, 255 μg/kg, 260 μg/kg, 265 μg/kg, 270 μg/kg, 275 μg/kg, 280 μg/kg, 285 μg/kg, 290 μg/kg, 295 μg/kg, 300 μg/kg, 305 μg/kg, 310 μg/kg, 315 μg/kg, 320 μg/kg, 325 μg/kg, 330 μg/kg, 335 μg/kg, 340 μg/kg, 345 μg/kg, 350 μg/kg, 355 μg/kg, 360 μg/kg, 365 μg/kg, 370 μg/kg, 375 μg/kg, 380 μg/kg, 385 μg/kg, 390 μg/kg, 395 μg/kg, 400 μg/kg, 405 μg/kg, 410 μg/kg, 415 μg/kg, 420 μg/kg, 425 μg/kg, 430 μg/kg, 435 μg/kg, 440 μg/kg, 445 μg/kg, 450 μg/kg, 455 μg/kg, 460 μg/kg, 465 μg/kg, 470 μg/kg, 475 μg/kg, 480 μg/kg, 485 μg/kg, 490 μg/kg, 495 μg/kg, or 500 μg/kg. In some embodiments, the CXCR4 antagonist (e.g., plerixafor or a pharmaceutically acceptable salt thereof) is administered to the donor at a dose of from about 200 μg/kg to about 300 μg/kg, such as a dose of about 240 μg/kg.

For example, in some embodiments, the CXCR4 antagonist (e.g., plerixafor or a pharmaceutically acceptable salt thereof) is administered to the donor at a dose of from about 50 μg/kg per day to about 500 μg/kg per day, such as a dose of about 50 μg/kg per day, 55 μg/kg per day, 60 μg/kg per day, 65 μg/kg per day, 70 μg/kg per day, 75 μg/kg per day, 80 μg/kg per day, 85 μg/kg per day, 90 μg/kg per day, 95 μg/kg per day, 100 μg/kg per day, 105 μg/kg per day, 110 μg/kg per day, 115 μg/kg per day, 120 μg/kg per day, 125 μg/kg per day, 130 μg/kg per day, 135 μg/kg per day, 140 μg/kg per day, 145 μg/kg per day, 150 μg/kg per day, 155 μg/kg per day, 160 μg/kg per day, 165 μg/kg per day, 170 μg/kg per day, 175 μg/kg per day, 180 μg/kg per day, 185 μg/kg per day, 190 μg/kg per day, 195 μg/kg per day, 200 μg/kg per day, 205 μg/kg per day, 210 μg/kg per day, 215 μg/kg per day, 220 μg/kg per day, 225 μg/kg per day, 230 μg/kg per day, 235 μg/kg per day, 240 μg/kg per day, 245 μg/kg per day, 250 μg/kg per day, 255 μg/kg per day, 260 μg/kg per day, 265 μg/kg per day, 270 μg/kg per day, 275 μg/kg per day, 280 μg/kg per day, 285 μg/kg per day, 290 μg/kg per day, 295 μg/kg per day, 300 μg/kg per day, 305 μg/kg per day, 310 μg/kg per day, 315 μg/kg per day, 320 μg/kg per day, 325 μg/kg per day, 330 μg/kg per day, 335 μg/kg per day, 340 μg/kg per day, 345 μg/kg per day, 350 μg/kg per day, 355 μg/kg per day, 360 μg/kg per day, 365 μg/kg per day, 370 μg/kg per day, 375 μg/kg per day, 380 μg/kg per day, 385 μg/kg per day, 390 μg/kg per day, 395 μg/kg per day, 400 μg/kg per day, 405 μg/kg per day, 410 μg/kg per day, 415 μg/kg per day, 420 μg/kg per day, 425 μg/kg per day, 430 μg/kg per day, 435 μg/kg per day, 440 μg/kg per day, 445 μg/kg per day, 450 μg/kg per day, 455 μg/kg per day, 460 μg/kg per day, 465 μg/kg per day, 470 μg/kg per day, 475 μg/kg per day, 480 μg/kg per day, 485 μg/kg per day, 490 μg/kg per day, 495 μg/kg per day, or 500 μg/kg per day. In some embodiments, the CXCR4 antagonist (e.g., plerixafor or a pharmaceutically acceptable salt thereof) is administered to the donor at a dose of from about 200 μg/kg per day to about 300 μg/kg per day, such as a dose of about 240 μg/kg per day. In some embodiments, the CXCR4 antagonist may be administered as a single dose. In other embodiments, the CXCR4 antagonist may be administered as two or more doses.

In various embodiments, the CXCR4 antagonist can be administered at a lower dose for a donor who exhibits renal dysfunction. For example, donors not exhibiting renal dysfunction may be administered at a dose of from about 200 μg/kg per day to about 300 μg/kg per day, such as a dose of about 240 μg/kg per day. In various embodiments, donors exhibiting renal dysfunction may be administered the CXCR4 antagonist at a lower dose, such as a dose of from about 100 μg/kg per day to about 200 μg/kg per day. In various embodiments, donors exhibiting renal dysfunction may be administered the CXCR4 antagonist at a dose of from about 120 μg/kg per day to about 200 μg/kg per day. In various embodiments, donors exhibiting renal dysfunction may be administered the CXCR4 antagonist at a dose of from about 140 μg/kg per day to about 180 μg/kg per day. In various embodiments, donors exhibiting renal dysfunction may be administered the CXCR4 antagonist at a dose of from about 150 μg/kg per day to about 170 μg/kg per day. In various embodiments, donors exhibiting renal dysfunction may be administered the CXCR4 antagonist at a dose of from about 155 μg/kg per day to about 165 μg/kg per day. In various embodiments, donors exhibiting renal dysfunction may be administered the CXCR4 antagonist at a dose of about 160 μg/kg per day.

In various embodiments, renal dysfunction in the donor is measured according to a reduction in glomerular filtration rate (GFR). Patients with chronic kidney disease are classified into stages based upon their glomerular filtration rate (GFR). The GFR for stage 1 patients is ≥90 mL/minute/1.73 m² (considered normal), stage 2 is 60-89 mL/minute/1.73 m² (considered mild), stage 3 is 30-59 mL/minute/1.73 m² (considered mild to moderate), stage 4 is 15-29 mL/minute/1.73 m² (considered severe), and stage 5 is <15 mL/minute/1.73 m² (kidney failure). In various embodiments, a donor categorized in any of mild, moderate, severe, or kidney failure categories is considered to have renal dysfunction. In various embodiments, renal dysfunction in the donor is measured according to creatine clearance. In some embodiments, renal dysfunction is present in a donor if the donor exhibits creatine clearance of <50 m/min.

Contemplated CXCR2 agonists and optionally CXCR4 antagonists may be administered to a subject in one or more doses. For example, a CXCR2 agonist and optionally a CXCR4 antagonist may be administered as a single dose or in two, three, four, five, or more doses. When multiple doses are administered, subsequent doses may be provided during the same day or one or more days, weeks, months, or years following the initial dose. For instance, the contemplated CXCR2 agonists and optionally CXCR4 antagonists described herein may be administered to a subject, such as a human subject one or more times daily, weekly, monthly, or yearly, depending on such factors as, for instance, the subject's age, body weight, sex, the subject's diet, and the subject's excretion rate.

In certain embodiments, the contemplated CXCR2 agonists and optionally CXCR4 antagonists are each administered in a single dose once per day. In certain embodiments, the contemplated CXCR2 agonists and optionally CXCR4 antagonists are each administered on two consecutive days. In certain embodiments, the contemplated CXCR2 agonists and CXCR4 antagonists are each administered in a single dose once per day on two consecutive days. In certain embodiments, administration of the contemplated CXCR2 agonists and optionally CXCR4 antagonists on two consecutive days improves the yield of CD34⁺ cells from the donor. In certain embodiments, administration of the contemplated CXCR2 agonists and optionally CXCR4 antagonists on two consecutive days improves the yield of neutrophils from the patient. In certain embodiments, administration of the contemplated CXCR2 agonists and optionally CXCR4 antagonists on two consecutive days allows for sufficient numbers of CD34⁺ cells to be obtained from a donor for transplantation, where administration on one day is insufficient. In certain embodiments, the donor may have a condition which results in insufficient mobilization of stem cells from the bone marrow.

In various embodiments, the contemplated CXCR2 agonists and CXCR4 antagonists are administered to a donor on a first day, apheresis is performed on the same day to generate an apheresis product, and the apheresis product is analyzed to determine whether the donor is to further undergo the CXCR2 agonist and CXCR4 antagonist dosing regimen on a second day. In various embodiments, the donor further undergoes the CXCR2 agonist and CXCR4 antagonist dosing regimen on a second day if the apheresis product includes less than a threshold amount of stem cells e.g., CD34+ cells/kg. In various embodiments, the threshold amount of stem cells e.g., CD34+ cells/kg is about 1×10⁶ cells/kg body weight of the donor. In various embodiments, the threshold amount of stem cells e.g., CD34+ cells/kg is about 2×10⁶ cells/kg, is about 3×10⁶ cells/kg, is about 4×10⁶ cells/kg, is about 5×10⁶ cells/kg, is about 6×10⁶ cells/kg, is about 7×10⁶ cells/kg, is about 8×10⁶ cells/kg, is about 9×10⁶ cells/kg, or is about 10×10⁶ cells/kg body weight of the donor. In particular embodiments, the threshold amount of stem cells e.g., CD34+ cells/kg is about 4×10⁶ cells/kg. In particular embodiments, the threshold amount of stem cells e.g., CD34+ cells/kg is about 6×10⁶ cells/kg.

to about 6×10⁶ cells/kg body weight of the recipient. As used herein, unless indicated otherwise, units expressed as cells/kg refers to cells/kg body weight of a recipient. In certain embodiments, the recipient is about 70 kg. In certain embodiments, the recipient is about 5 kg to about 150 kg. In certain embodiments, CD34+ cells are present in an amount of from about 1×10⁶ cells/kg to about 6×10⁶ cells/kg. In certain embodiments, CD34+ cells are present in an amount of from about 2×10⁶ cells/kg to about 5.75×10⁶ cells/kg, from about 3×10⁶ cells/kg to about 5.5×10⁶ cells/kg, from about 3.5×10⁶ cells/kg to about 5.25×10⁶ cells/kg, from about 4.0×10⁶ cells/kg to about 5.0×10⁶ cells/kg, from about 4.25×10⁶ cells/kg to about 4.75×10⁶ cells/kg, or from about 4.4×10⁶ cells/kg to about 4.6×10⁶ cells/kg

In certain embodiments, administration of the CXCR2 agonists and CXCR4 antagonists results in a minimal change in neutrophil activation markers. Examples of neutrophil activation markers include L-selectin, CD11b, CD18, and CD66. In contrast G-CSF, the traditional therapy of choice for mobilization of neutrophils, enhances neutrophil activation, which is problematic, for example, in patients with sickle cell disease where activated neutrophils adhere to the endothelium, thereby increasing the risk of severe and life-threatening complications such as vaso-occlusive crises. Accordingly, in certain embodiments, administration of a CXCR2 agonist and/or CXCR4 antagonists results in less than a 2.5 fold change (e.g., less than a 2 fold change, less than a 1.5 fold change or less than a 1 fold change) in a neutrophil activation marker, such as L-selectin, CD11b, CD18, or CD66.

Hematopoietic stem or progenitor cells and pharmaceutical compositions described herein may be administered to a subject in one or more doses. When multiple doses are administered, subsequent doses may be provided one or more days, weeks, months, or years following the initial dose. For instance, the hematopoietic stem cells and pharmaceutical compositions described herein may be administered to a subject, such as a human subject suffering from one or more diseases, conditions, or disorders described herein, one or more times daily, weekly, monthly, or yearly, depending on such factors as, for instance, the subject's age, body weight, sex, severity of the diseases being treated, the subject's diet, and the subject's excretion rate.

### EXAMPLES

The following examples are put forth so as to provide those of ordinary skill in the art with a description of how the compositions and methods described herein may be used, made, and evaluated, and are intended to be purely exemplary and are not intended to limit the scope of the invention.

Mobilized peripheral blood grafts are currently the predominant source of hematopoietic stem and progenitor cells (HSPC) for both autologous and allogeneic transplantation. The most common clinical hematopoietic stem cell mobilization protocol is five days of Filgrastim (G-CSF). This regimen requires daily injections, has been associated with bone pain and often results in unpredictably low yields. In this example, data are presented to demonstrate particular treatment protocols using MGTA-145 and plerixafor, which induces increased mobilization of CD34+ cells, CD34+CD90+CD45RA− cells, neutrophils, and white blood cells (WBCs).

### Example 1: Phase 1 Clinical Trial Methods

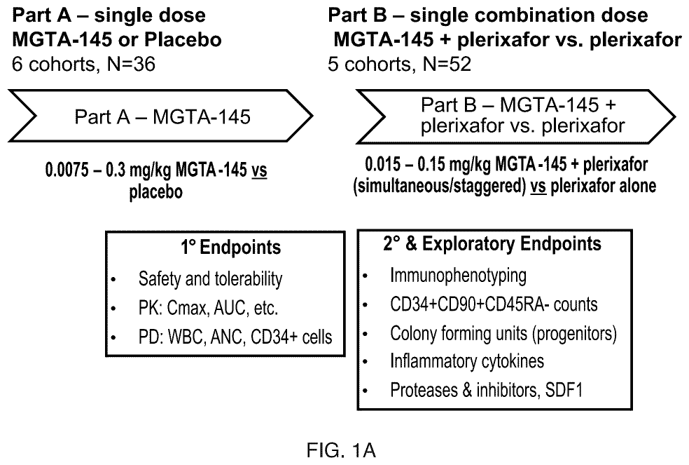

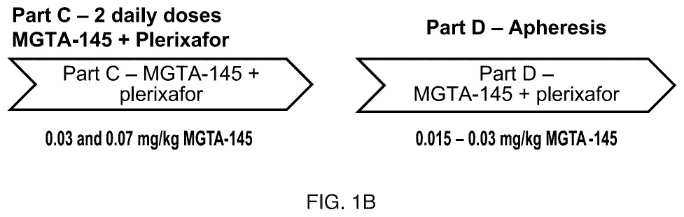

FIG. 1A depicts the treatments provided to patient cohorts enrolled in Part A and Part B of a Phase 1 clinical trial as well as measurable endpoints. In particular, Part A of the Phase 1 trial involved administering a single dose of MGTA-145 or a placebo into 6 patient cohorts (N=36 patients). The dose of MGTA-145 was one of 0.0075 mg/kg, 0.015 mg/kg, 0.03 mg/kg, 0.075 mg/kg, 0.15 mg/kg, or 0.3 mg/kg. Part B of the Phase 1 trial involved administering a combination dose of MGTA-145 and plerixafor (administered either simultaneously or with the dose of MGTA-145 administered 2 hours after the dose of plerixafor—“staggered 2 hours administration protocol”) or plerixafor alone across 5 patient cohorts (N=52). The dose of MGTA-145 was one of 0.015 mg/kg, 0.03 mg/kg, 0.075 mg/kg, or 0.15 mg/kg. The dose of plerixafor was 240 μg/kg (0.24 mg/kg). FIG. 1B depicts the treatments provided to patient cohorts enrolled in Part C and Part D. Part C of the Phase 1 trial involved administering a combination dose of MGTA-145 and plerixafor (staggered 2 hour administration protocol) or plerixafor alone on two consecutive days across three patient cohorts (N=10). The dose of MGTA-145 was one of 0.03 mg/kg or 0.075 mg/kg. The dose of plerixafor was 240 μg/kg (0.24 mg/kg). Part D involved administering a combination dose of MGTA-145 and plerixafor (MGTA-145 administered 2 hours after administration of plerixafor) across 9 patients followed by performing an apheresis procedure to obtain apheresis products from the patients. The dose of MGTA-145 was 0.015 mg/kg or 0.03 mg/kg and the dose of plerixafor was 240 Ng/kg (0.24 mg/kg). Additional details of the characteristics of patients enrolled in Parts A-D of the Phase 1 clinical trial as well as the treatments that each patient received are described below in TABLES 4A and 4B.

TABLE 4A
Characteristics of patients enrolled in Part A
and Part B of MGTA-145 Phase 1 clinical trial

| | | | | | Part B | | | |
|---|---|---|---|---|---|---|---|---|
| | Part A | | | | MGTA-145 + | | | |
| | MGTA-145 | | Placebo | | plerixafor | | Plerixafor | |
| | N = 24 | | N = 12 | | N = 38 | | N = 14 | |
| Age, years (range) | 43 | (27-59) | 40 | (22-54) | 39 | (22-59) | 37 | (18-59) |
| Male (%) | 20 | (83) | 8 | (67) | 30 | (79) | 11 | (79) |
| Weight, kg (range) | 85 | (57-111) | 83 | (59-97) | 82 | (54-107) | 78 | (58-106) |

| | | | | |
|---|---|---|---|---|
| Race, n | 14 | 5 | 12 | 6 |
| White | | | | |
| Black/AA | 7 | 6 | 24 | 7 |
| Other | 3 | 1 | 2 | 1 |

TABLE 4B
Characteristics of patients enrolled in Part C
and Part D of MGTA-145 Phase 1 clinical trial

| | Part C | | | | Part D | |
|---|---|---|---|---|---|---|
| | MGTA-145 + | | | | MGTA-145 + | |
| | plerixafor | | Plerixafor | | plerixafor | |
| | N = 8 | | N = 2 | | N = 9* | |
| Age, years (range) | 35 | (24-57) | 35 | (24-41) | 38 | (19-54) |
| Male (%) | 8 | (100) | 1 | (50) | 7 | (78) |
| Weight, kg (range) | 77 | (63-97) | 77 | (63-88) | 81 | (72-94) |

| | | | |
|---|---|---|---|
| Race, n | 4 | 1 | 7 |
| White | | | |
| Black/AA | 4 | 1 | 2 |
| Other | 0 | 0 | 0 |

*A 9^{th} subject enrolled in Part D but did not undergo leukapheresis.

Patients were treated with their respective treatments (e.g., placebo, MGTA-145, plerixafor, or MGTA-145+plerixafor). Patients treated with the combination therapy of MGTA-145 and plerixafor either underwent simultaneous dosing of the combination therapy or staggered dosing (MGTA-145 given two hours after plerixafor).

A peripheral blood sample was collected from patients immediately prior to and 0.5, 1, 2, 4, 6, 8, 12, and 24 hours after administration of both agents (for simultaneous dosing) and after administration of MGTA-145 (staggered dosing) and analyzed by multicolor flow cytometry to quantitate mobilization of CD34+ cells, CD34+CD40+CD45RA− cells, T cells, B cells, and MDSCs. Mobilization of white blood cells (WBCs), neutrophils, lymphocytes, basophils, and eosinophils was quantified by automated hematology analyzer. Additionally, blood samples were analyzed by multicolor flow cytometry to quantify changes in expression of neutrophil activation markers such as L-selectin, CD11b, CD18, and CD66. Additionally, matrix metalloproteinase 9 (MMP-9), a neutrophil protease believed to mediate mobilization in response to MGTA-145, as well as its cognate inhibitor, TIMP-1, were quantified in plasma samples collected at the same time points by sandwich immunoassay.

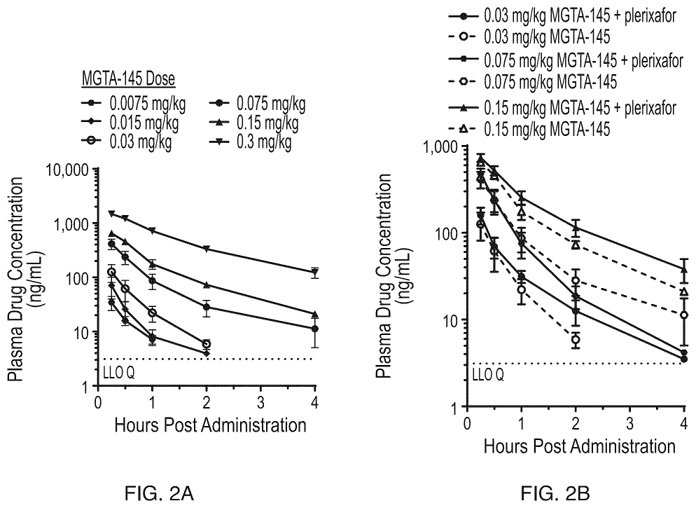

Pharmacokinetics of MGTA-145 was also assessed by determining the plasma drug concentration immediately prior to and at 1, 2, 3, and 4 hours post-administration. Plasma concentrations of MGTA-145 following single dose administration (0.0075-0.3 mg/kg) as monotherapy are shown in FIG. 2A. Plasma concentrations of MGTA-145 following single dose administration (0.03-0.15 mg/kg) in combination with a single dose of plerixafor (0.24 mg/kg) are shown in FIG. 2B. Data represent at least 4 subjects per dose level and are expressed as mean+/−SEM.

Patients were monitored for emergent adverse events. TABLE 5 shows the emergent adverse events exhibited by patients enrolled in Parts A-C of the MGTA-145 Phase 1 clinical trial.

TABLE 5
Treatment Emergent Adverse Events¹

| | Part A | | Part B | | Part C | | Part D |
|---|---|---|---|---|---|---|---|
| | MGTA- | | MGTA- | | MGTA- | | MGTA- |
| | 145 | | 145 + | | 145 + | | 145 + |
| | (0.0075- | | plerixafor | | plerixafor | | plerixafor |
| | 0.3 | | (0.015-0.15 | | (0.03- | | (0.015- |
| | mg/kg) | Placebo | mg/kg) | Plerixafor | 0.075) | Plerixafor | 0.03) |
| | n = 24 | n = 12 | n = 38 | n = 14 | n = 8 | n = 2 | n = 9 |
| | n (%) | n (%) | n (%) | n (%) | n (%) | n (%) | n (%) |
| Subjects | 19 (79.2) | — | 31 (81.6) | 8 (57.1) | 6 (75.0) | — | 8 (88.9) |
| with | | | | | | | |
| any drug | | | | | | | |
| related | | | | | | | |
| TEAE | | | | | | | |
| Diarrhea | — | — | 6 (15.8) | 5 (35.7) | 1 (12.5) | — | 1(11.1) |
| Nausea | — | — | 7 (18.4) | 2 (14.3) | 1 (12.5) | — | 4 (44.4) |
| Abdominal | — | — | 5 (13.2) | 4 (28.6) | — | — | 2 (22.2) |
| discomfort/ | | | | | | | |
| pain | | | | | | | |
| Vomiting | — | — | 3 (7.9) | 1 (7.1) | — | — | 1 (11.1) |
| Back pain/ | 19 (79.2) | — | 24 (63.2) | 2 (14.3) | 4 (50.0) | — | 3 (33.3) |
| Musculo- | | | | | | | |
| skeletal | | | | | | | |
| pain² | | | | | | | |
| Dizziness | — | — | 5 (15.6) | 1 (7.1) | — | — | 3 (33.3) |
| Headache | — | — | 4 (10.5) | 1 (7.1) | 1 (12.5) | — | 2 (22.2) |
| Dysgeusia | — | — | — | 2 (14.3) | — | — | — |
| Paraesthesia | — | — | 2 (5.3) | — | 1 (12.5) | — | 1(11.1) |

¹All adverse events (AEs) were grade 1 except for grade 2 abdominal pain (1), nausea (1), and back pain (1) in the plerixafor + MGTA-145 0.075 mg/kg 2 h stagger cohort (Part B) and grade 2 headache (1) in the plerixafor + MGTA-145 0.015 mg/kg cohort (Part D). There was no dose response in AEs, so data are aggregated.
²Back pain was associated with MGTA-145 infusion, lasted <20 minutes in most cases and did not require medical therapy.

### Example 2: MGTA-145 Monotherapy Mobilizes White Blood Cells, Neutrophils, CD34+ Cells, and CD34+CD90+CD45RA− Cells

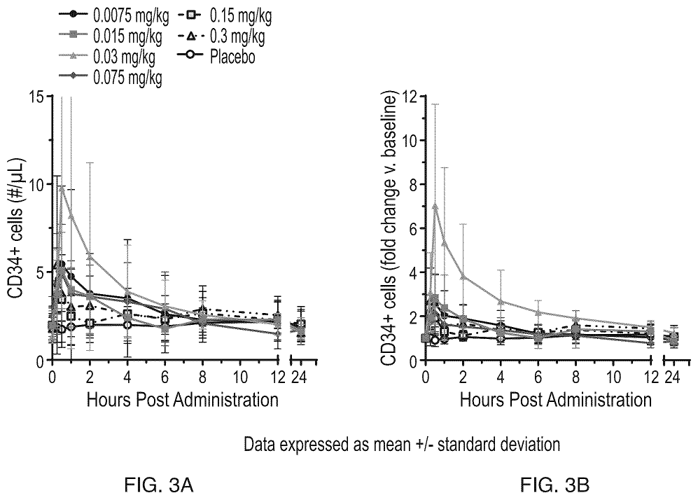

FIG. 3A shows the mobilization of CD34⁺ cells over the course of 24 hours following MGTA-145 monotherapy. FIG. 3B shows the fold change of CD34⁺ cells over the course of 24 hours following MGTA-145 monotherapy. Generally, across the six MGTA-145 doses, peak CD34+ mobilization occurred about 30 minutes post administration followed by a decline over the course of 24 hours. The MGTA-145 monotherapy induced increases in CD34⁺ cells across all six doses. Notably, a 0.03 mg/kg MGTA-145 dose achieved the highest peak concentration of CD34⁺ cells (˜10 cells/μL) in comparison to the other doses which achieved ˜5 cells/μL. Expressed in terms of fold increase, the 0.03 mg/kg MGTA-145 dose increased CD34⁺ cells by 7-fold whereas the other doses increased CD34⁺ cells by 2-3 fold.

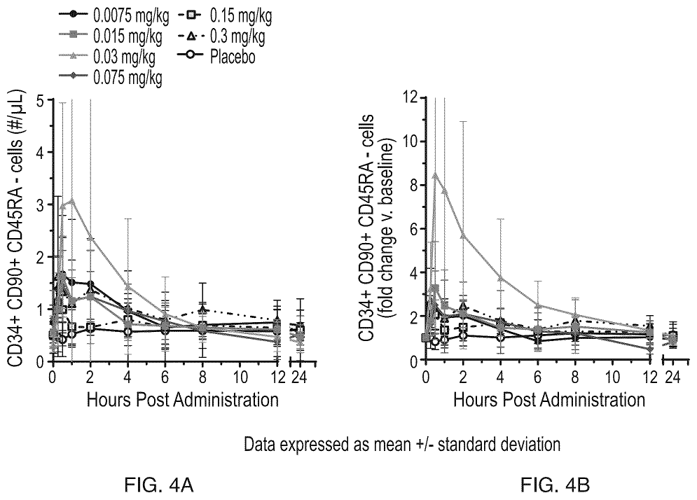

FIG. 4A shows the mobilization of CD34⁺CD90⁺CD45RA⁻ cells over the course of 24 hours following MGTA-145 monotherapy. FIG. 4B shows the fold change of CD34⁺CD90⁺CD45RA⁻ cells over the course of 24 hours following MGTA-145 monotherapy. CD34⁺CD90⁺CD45⁻ cells are indicative of a stem cell phenotype associated with long term engraftment. The MGTA-145 monotherapy induced statistically significant increases in CD34⁺CD90⁺CD45RA⁻ cells across all six doses. Peak CD34⁺CD90⁺CD45RA⁻ mobilization occurred 30 minutes post administration followed by a decline towards initial concentrations over the course of 24 hours. Notably, a 0.03 mg/kg MGTA-145 dose achieved the highest peak concentration of CD34⁺CD90⁺CD45RA⁻ cells (˜3 cells/μL) in comparison to the other doses which achieved ˜1-1.6 cells/μL. Expressed in terms of fold increase, the 0.03 mg/kg MGTA-145 dose increased CD34⁺CD90⁺CD45RA⁻ cells by ˜8.3-fold whereas the other doses increased CD34⁺CD90⁺CD45RA⁻ cells by 2-3.5 fold.

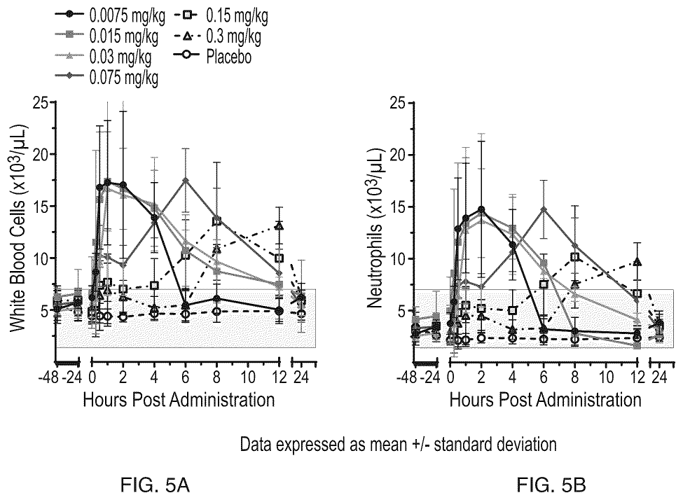

FIGS. 5A and 5B show the mobilization of WBCs and neutrophils, respectively, over the course of 24 hours following MGTA-145 administration. Generally, for the lower MGTA-145 doses (e.g., 0.0075 mg/kg, 0.015 mg/kg, 0.03 mg/kg), peak mobilization of neutrophils (˜13-15×10³ cells/μL) and WBCs (˜15-17×10³ cells/μL) occurred between 1 and 2 hours post administration. For the higher MGTA-145 doses (e.g., 0.075 mg/kg, 0.15 mg/kg, and 0.3 mg/kg), an initial plateau of neutrophil (˜5-8×10³ cells/μL) and WBC (˜6-10×10³ cells/μL) mobilization was observed at 2 hours post administration, followed by a subsequent increase in mobilization at 6-8 hours post administration. The subsequent increase correlated with re-expression of CXCR2 and presence of MGTA-145 at pharmacologically active levels. Altogether, FIGS. 5A and 5B depict the rapid mobilization of neutrophils and WBCs at all six doses of MGTA-145 monotherapy.

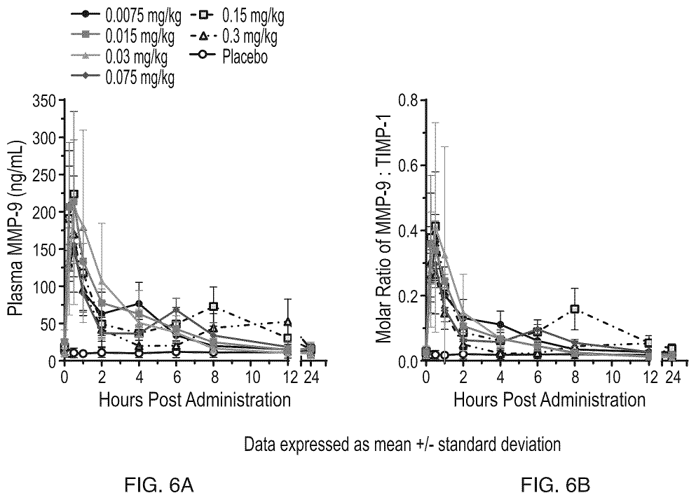

FIGS. 6A and 6B show the plasma levels of MMP-9 and molar ratio of MMP-9:TIMP-1, respectively, over the course of 24 hours following MGTA-145 monotherapy. MMP-9 is an enzyme involved in the breakdown of extracellular matrix and indicative of neutrophil migration whereas TIMP-1 blocks MMP-9 mediated migration. Therefore, an increase in MMP-9 or an increase in MMP-9:TIMP-1 ratio is indicative of increased neutrophil mobilization. As shown in FIG. 6A, plasma levels of MMP-9 increased sharply within 30 minutes post-administration across all six doses of MGTA-145 monotherapy. The plasma levels of MMP-9 then decreased towards initial concentrations over the course of 24 hours. The MMP-9:TIMP-1 ratio exhibited a similar trend (FIG. 6B), with a rapid increase 30 minutes post-administration followed by a decrease towards initial concentrations over the course of 24 hours. Notably, the changes in plasma levels of MMP-9 and the molar ratio of MMP-9:TIMP-1 do not significantly differ across the six MGTA-145 doses, likely suggesting a saturation of response at even the lowest dose (e.g., 0.0075 mg/kg).

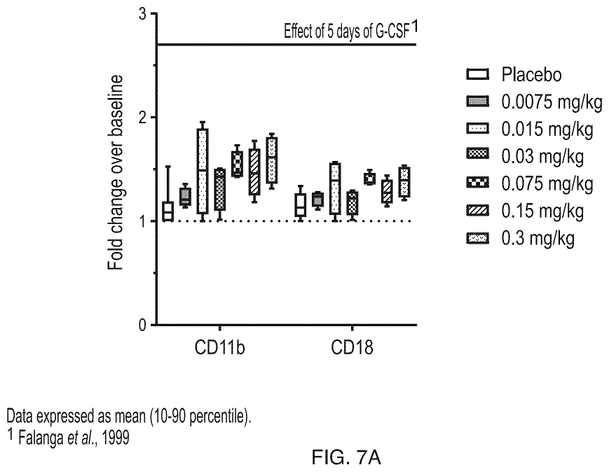

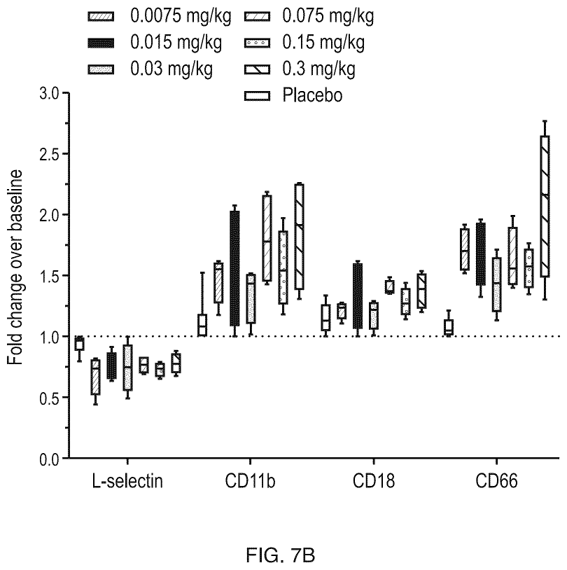

FIG. 7A shows the limited change in neutrophil activation markers (CD11b and CD18) following MGTA-145 monotherapy. G-CSF, the traditional therapy of choice for mobilization of neutrophils, enhances neutrophil activation, as measured by CD11b and CD18, but this has been identified to be problematic, for example, in patients with sickle cell disease where activated neutrophils adhere to the endothelium, thereby increasing the risk of severe and life-threatening complications such as vaso-occlusive crises. FIG. 7A depicts that 5 days of G-CSF treatment results in a ˜2.7 fold increase in neutrophil activation markers as reported by Falanga et al., Blood, 93(8), 1999. In contrast, the administration of MGTA-145 monotherapy (across all six doses) induces less than a 2-fold change relative to baseline for the neutrophil activation markers of CD11b and CD18. This suggests that MGTA-145 is an improved alternative to G-CSF particularly, for example, in the context of patients with sickle cell disease.

Additionally, FIG. 7B also shows the limited change in neutrophil activation markers (L-selectin, CD11b, CD18, and CD66) following MGTA-145 monotherapy. Similar to the results shown in FIG. 7A, administration of MGTA-145 monotherapy (across all six doses) induces less than a 2-fold change relative to baseline for the neutrophil activation markers of CD11b and CD18. Furthermore, administration of MGTA-145 monotherapy (across all six doses) induces less than a 2-fold change relative to baseline for the neutrophil activation marker of L-selectin. Except for the highest dose of MGTA-145 (e.g., 0.3 mg/kg), the MGTA-145 monotherapy induces less than a 2-fold change relative to baseline for the neutrophil activation marker of CD66.

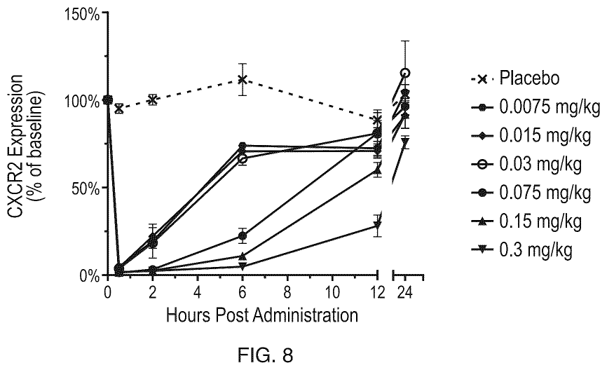

FIG. 8 shows that MGTA-145 monotherapy leads to rapid downregulation of its target receptor, CXCR2, on peripheral neutrophils, followed by recovery over 24 hours.

### Example 3: Simultaneous Combination Therapy of MGTA-145 and Plerixafor

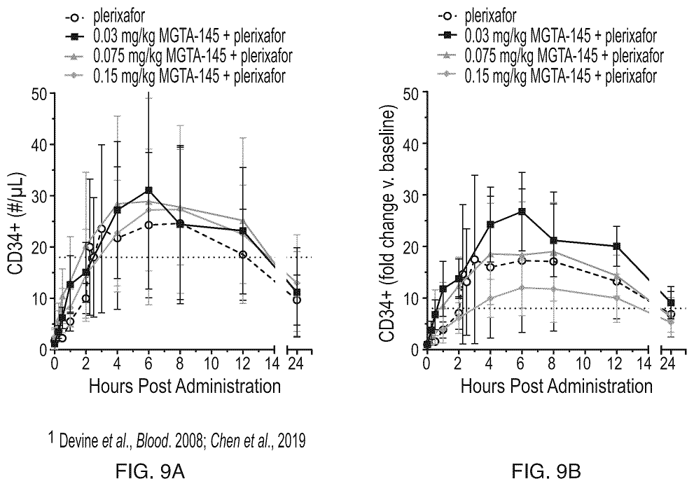

FIG. 9A shows the mobilization of CD34+ cells over the course of 24 hours following simultaneous combination treatment of MGTA-145 and plerixafor. FIG. 9B shows the fold change of CD34+ cells over the course of 24 hours following simultaneous combination treatment of MGTA-145 and plerixafor. Additionally for comparison purposes, FIGS. 9A and 9B depict the peak mobilization levels of CD34+ cells in response to plerixafor alone (see dotted line), as detailed in Devine et al. (2008) Blood 112(4): 990-998 and Chen et al. (2019) Blood Adv. (2019) 3(6):875-883. Across the three MGTA-145 doses in combination with plerixafor, peak CD34+ mobilization occurred 6-8 hours post administration followed by a decline towards initial concentrations over the course of 24 hours. The peak CD34+ mobilization induced by this combination therapy was higher than plerixafor alone. A 0.03 mg/kg MGTA-145 dose+plerixafor achieved the highest peak concentration of CD34+ cells (˜30 cells/μL) at 6 hours post administration. Expressed in terms of fold increase over baseline, the 0.03 mg/kg MGTA-145 dose+plerixafor increased CD34+ cells by ˜27-fold whereas the 0.075 mg/kg MGTA-145 dose+plerixafor and the 0.15 mg/kg MGTA-145 dose+plerixafor increased CD34+ cells by ˜21-fold and ˜11-fold respectively. TABLE 6 shows the quantified CD34+ cell levels in response to plerixafor alone versus simultaneous MGTA-145+plerixafor treatment across three different MGTA-145 doses (0.03 mg/kg, 0.075 mg/kg, and 0.15 mg/kg). Specifically, TABLE 6 shows the median peak CD34+ cells/μL level and the median fold change in peak CD34+ cells/μL over baseline for patients receiving each treatment. Generally, patients that received the combination of MGTA-145 and plerixafor exhibited higher peak CD34+ levels and higher fold change over baseline in comparison to patients that received plerixafor alone. In particular, patients that received 0.03 mg/kg MGTA-145+plerixafor exhibited a 1.3 fold increase in median peak CD34+ levels in comparison to patients that received plerixafor alone.

TABLE 6
CD34+ cell levels in response to plerixafor alone
versus simultaneous MGTA-145 + plerixafor

| | | | Peak CD34+/μL |
|---|---|---|---|
| MGTA-145 dose | | Peak CD34+/μL | (FC vs. baseline) |
| (mg/kg) | Regimen | Median (range) | Median (range) |
| 0 | Plerixafor | 20 (13-65) | 15 (8-62) |
| | alone | | |
| 0.03 | Simultaneous | 26 (13-65) | 27 (17-36) |
| 0.075 | MGTA-145 + | 27 (13-67) | 21 (11-31) |
| 0.15 | plerixafor | 29 (9-55) | 11 (8-22) |

TABLE 7 shows the breakdown of peak CD34+ mobilization across patients that received plerixafor alone versus simultaneous MGTA-145+plerixafor treatment across three different MGTA-145 doses (0.03 mg/kg, 0.075 mg/kg, and 0.15 mg/kg). Specifically, the CD34+ levels for each patient were analyzed to determine the percentage of patients in each dose group to achieve ≥20 and/or ≥40 CD34+ cells/μL at peak. Generally, a higher percentage of patients that received the combination of plerixafor and MGTA-145 (any of 0.03 mg/kg, 0.075 mg/kg, or 0.15 mg/kg) achieved a peak CD34+ count of ≥20 or ≥40 cells/μL in comparison to patients that received plerixafor alone. In particular, of the 18 patients receiving combination plerixafor and MGTA-145 (any of 0.03 mg/kg, 0.075 mg/kg, or 0.15 mg/kg), 15 patients achieved a CD34+ count of at least 20 cells/μL at peak. This represents a 1.7 fold increase in comparison to patients that received plerixafor alone. Similarly, of the 10 patients that received the combination plerixafor and MGTA-145 (any of 0.03 mg/kg, 0.075 mg/kg, or 0.15 mg/kg), 3 achieved a peak CD34+ count of at least 40 cells per microliter. This represents a 1.2 fold increase in comparison to patients that received plerixafor alone.

TABLE 7
Categorized patients according to their peak CD34+
cell levels in response to plerixafor alone versus simultaneous
MGTA-145 + plerixafor

| MGTA-145 | | | |
|---|---|---|---|
| dose (mg/kg) | Regimen | ≥20 (%) | ≥40 (%) |
| 0 | Plerixafor alone | 50% (7/14) | 14% (2/14) |
| 0.03 | Simultaneous | 83% (5/6) | 17% (1/6) |
| 0.075 | MGTA-145 + | 83% (5/6) | 17% (1/6) |
| 0.15 | plerixafor | 83% (5/6) | 17% (1/6) |
| All doses | | 83% (15/18) | 17% (3/18) |

| | | |
|---|---|---|
| Historical plerixafor alone data¹ | 28% (7/25) | 4% (1/25) |

¹Devine et al., Blood. 2008.

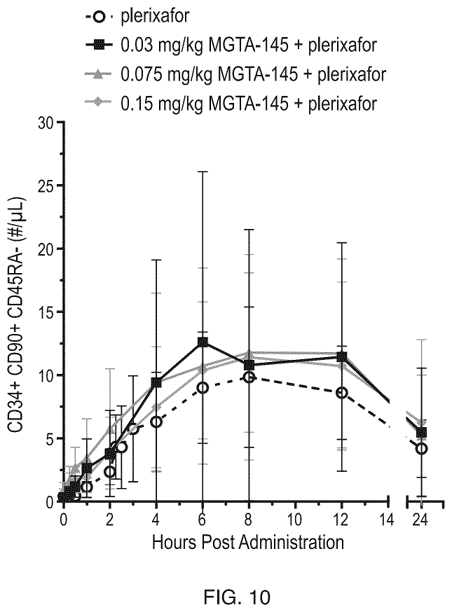

FIG. 10 shows the mobilization of CD34+CD90+CD45RA− cells over the course of 24 hours following MGTA-145+plerixafor therapy. For a 0.03 mg/kg MGTA-145 dose+plerixafor, peak CD34+CD90+CD45RA− mobilization (median ˜8 cells/μL) occurred about 6 hours post administration. The 0.075 mg/kg MGTA-145 dose+plerixafor and the 0.15 mg/kg MGTA-145 dose+plerixafor achieved peak CD34+CD90+CD45RA− mobilization (median ˜10-12 cells/μL) at 6-7 hours post administration.

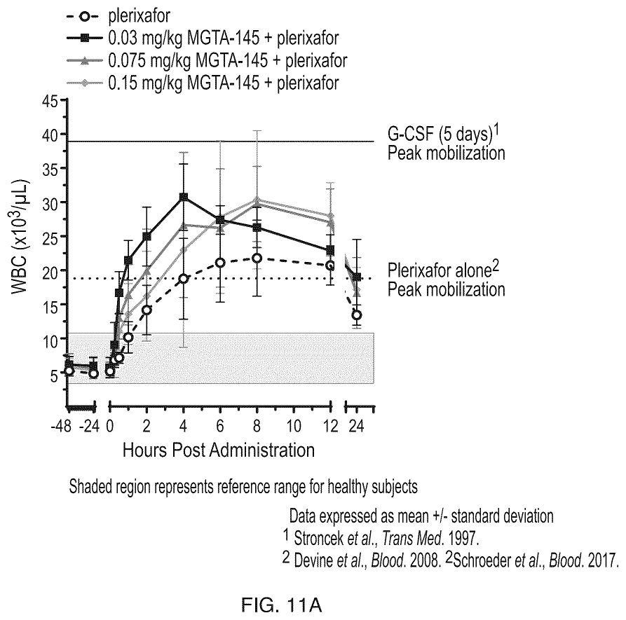

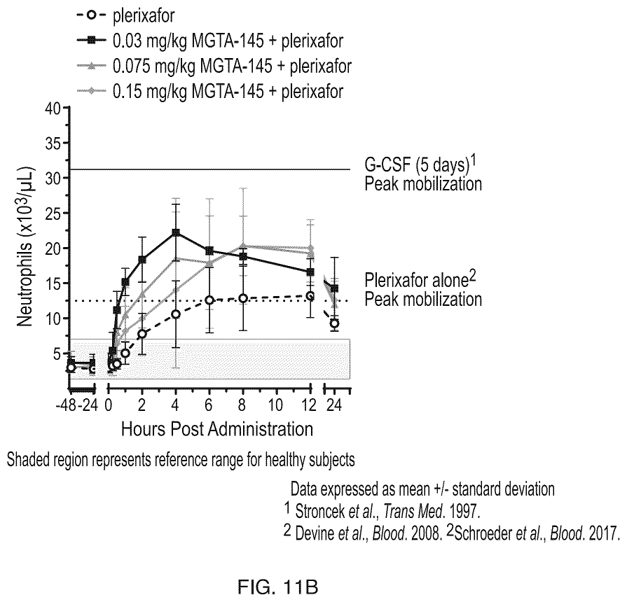

FIGS. 11A and 11B show the mobilization of neutrophils and WBCs, respectively, over the course of 24 hours following simultaneous treatment of MGTA-145 and plerixafor. Additionally for comparison purposes, FIGS. 11A and 11B depict the peak mobilization levels of neutrophils and WBCs in response to 5 days of G-CSF or plerixafor alone, as detailed in Stroncek et al. (1997) Trans Med. 7(1):19-24 (for G-CSF) and Devine et al. (2008, supra and Schroeder et al. (2017) Blood 129(19):2680-2692 (for plerixafor). Rapid mobilization of neutrophils and WBCs was observed across all three doses of MGTA-145 (0.03 mg/kg, 0.075 mg/kg, and 0.15 mg/kg) in combination with plerixafor. For combination therapy including 0.03 mg/kg MGTA-145 and plerixafor, peak neutrophil (˜22×10³ cells/μL) and WBC (˜30×10³ cells/μL) mobilization occurred at 4 hours post administration. For combination therapy including 0.075 mg/kg or 0.15 mg/kg MGTA-145 and plerixafor, peak neutrophil (˜20×10³ cells/μL) and WBC (˜30×10³ cells/μL) mobilization occurred at 8 hours post administration. Altogether, the combination therapy of MGTA-145 and plerixafor induces mobilization of neutrophils and WBCs beyond levels achievable by plerixafor alone; however, the combination therapy does not achieve the levels induced by 5 days of G-CSF.

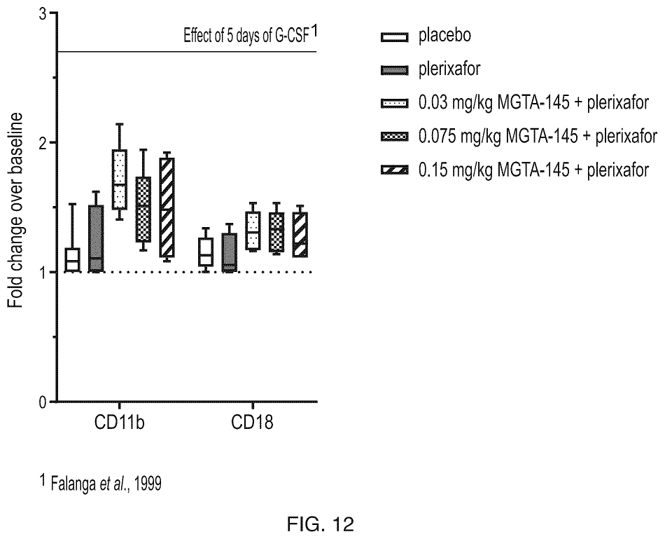

FIG. 12 shows the limited change in neutrophil activation markers (CD11b and CD18) following MGTA-145+plerixafor therapy. FIG. 12 further depicts the fold change over baseline resulting from 5 days of G-CSF (˜2.7 fold increase in neutrophil activation markers as reported by Falanga et al., (1999), supra). In contrast, the administration of MGTA-145 (across the three doses)+plerixafor induces less than a 2-fold change relative to baseline for the neutrophil activation markers of CD11b and CD18. This suggests that MGTA-145+plerixafor is an alternative to G-CSF particularly in the context of patients with sickle cell disease.

### Example 4: Staggered Combination Therapy of MGTA-145 and Plerixafor

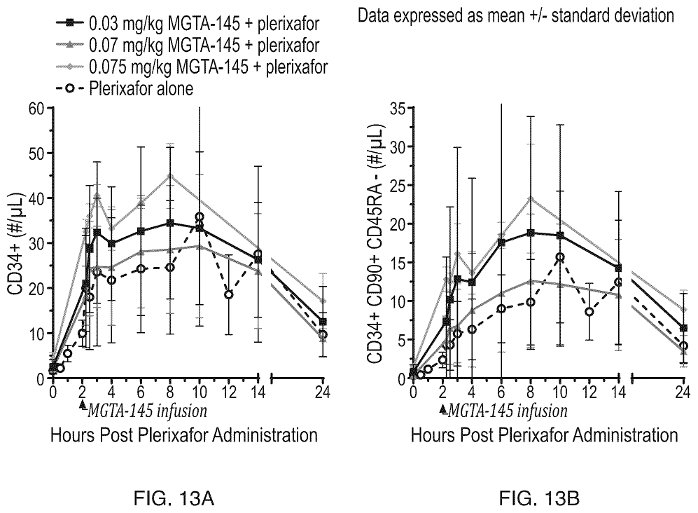

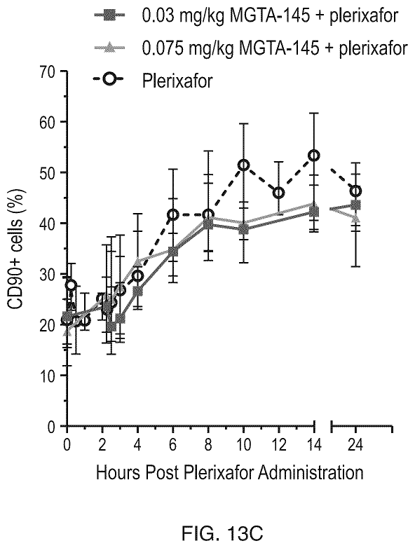

FIG. 13A depicts the mobilization of CD34⁺ cells in response to a staggered combination therapy at two doses of MGTA-145. The staggered combination treatment of 0.03 mg/kg MGTA-145 and plerixafor generally induced higher mobilization of CD34⁺ cells than 0.07/0.075 mg/kg MGTA-145+plerixafor or plerixafor alone. More specifically, the staggered administration of plerixafor followed by 0.03 mg/kg MGTA-145 induced a peak mobilization of CD34+ cells of ˜38×10³ cells/μL at a timepoint of 8 hours post plerixafor administration. The staggered administration of plerixafor followed by 0.07/0.075 mg/kg MGTA-145 induced a peak mobilization of CD34+ cells of ˜28×10³ cells/μL at a timepoint of 8 hours post plerixafor administration.

FIG. 13B depicts the mobilization of CD34⁺CD90⁺CD45RA⁻ cells in response to a staggered combination therapy at two doses of MGTA-145. The staggered combination treatment of 0.03 mg/kg MGTA-145 and plerixafor generally induced higher mobilization of CD34⁺CD90⁺CD45RA⁻ cells than 0.07/0.075 mg/kg MGTA-145+plerixafor or plerixafor alone. More specifically, the staggered administration of plerixafor followed by 0.03 mg/kg MGTA-145 induced a peak mobilization of CD34⁺CD90⁺CD45RA⁻ cells of ˜16×10³ cells/μL at a timepoint of 6 hours post plerixafor administration. The staggered administration of plerixafor followed by 0.07/0.075 mg/kg MGTA-145 induced a peak mobilization of CD34⁺CD90⁺CD45RA⁻ cells of ˜12×10³ cells/μL at a timepoint of 8 hours post plerixafor administration.

FIG. 13C is a graph showing the percentage of CD34+ cells that are CD34+CD90+CD45RA− cells following staggered combination therapy at two doses of MGTA-145. Following plerixafor and MGTA-145 combination therapy, there is an upward trend of the percentage of CD34+ cells that are CD34+CD90+CD45RA−, which represent engraftable HSCs. Specifically, about 20-25% of CD34+ cells are CD34+CD90+CD45RA− cells between 0-4 hours after plerixafor administration. The percentage of CD34+ cells that are CD34+CD90+CD45RA− then rises to ˜40% at 8-24 hours after plerixafor administration.

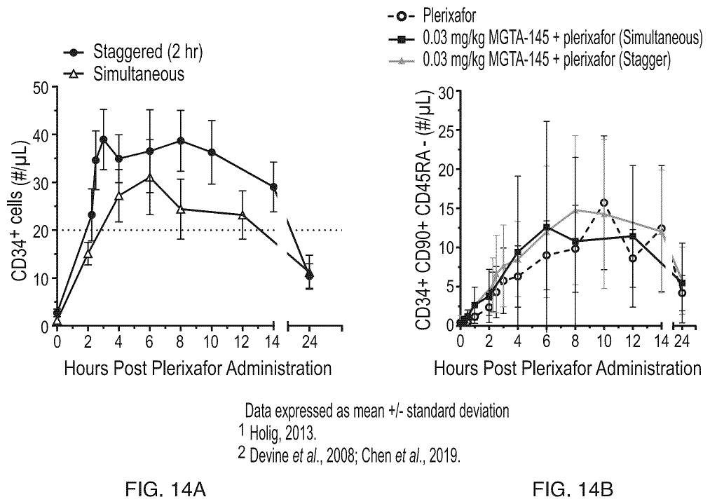

FIG. 14A compares the mobilization of CD34+ cells in response to either a simultaneous combination therapy or a staggered combination therapy (MGTA-145 given two hours after plerixafor). As shown in FIG. 14A, administration of 0.03 mg/kg MGTA-145 two hours after plerixafor administration resulted in increased mobilization of CD34+ cells (median peak: 36 cells/μL), which occurred 6 hours post plerixafor administration. This represents an increase in mobilization of CD34+ cells in comparison to both 1) the simultaneous administration of 0.03 mg/kg MGTA-145+plerixafor (median peak: 27 cells/μL), and 2) plerixafor alone (median peak: 20 cells/μL).

FIG. 14B compares the mobilization of CD34+CD90+CD45RA− cells in response to either a simultaneous combination therapy or a staggered combination therapy. As shown in FIG. 14B, administration of 0.03 mg/kg MGTA-145 two hours after plerixafor administration resulted in an increased mobilization of CD34+CD90+CD45RA− cells (median peak: 12 cells/μL), which occurred 6 hours post plerixafor administration. This represents an increase in mobilization of CD34+CD90+CD45RA− cells in comparison to both 1) the simultaneous administration of 0.03 mg/kg MGTA-145+plerixafor (median peak: 8 cells/μL), and 2) plerixafor alone (median peak: 8 cells/μL).

FIGS. 14A and 14B taken together suggest that the staggered administration of MGTA-145 and plerixafor represents a possible dosing regimen that induces higher levels of CD34+ and CD34+CD90+CD45RA− cells in comparison to simultaneous administration.

TABLE 8 shows the quantified CD34+ cell levels in response to simultaneous or staggered administration of MGTA-145+plerixafor. Specifically, TABLE 8 shows the median peak CD34+ cells/μL level, median CD34+ area under the curve (AUC) between 2-8 hours post plerixafor dose administration, and median time (Tmax) of the peak CD34+ level across patients that received one of 1) plerixafor alone, 2) simultaneous plerixafor and 0.03 mg/kg MGTA-145, 3) simultaneous plerixafor and 0.075 mg/kg MGTA-145, 4) simultaneous plerixafor and 0.15 mg/kg MGTA-145, 5) staggered plerixafor and 0.03 mg/kg MGTA-145, 6) staggered plerixafor and 0.070 mg/kg MGTA-145, and 7) staggered plerixafor and 0.07 mg/kg MGTA-145. Generally, patients that received staggered plerixafor and MGTA-145 exhibited higher peak CD34+ levels and higher CD34+ AUC in comparison to patients that received simultaneous administration of plerixafor and MGTA-145 and in comparison to patients that received plerixafor alone. In particular, patients that received staggered plerixafor and MGTA-145 (any of 0.03 mg/kg, 0.07 mg/kg, or 0.075 mg/kg) exhibited a 1.8 fold increase in median peak CD34+ levels in comparison to patients that received plerixafor alone. Additionally, patients that received staggered plerixafor and MGTA-145 (any of 0.03 mg/kg, 0.07 mg/kg, or 0.075 mg/kg) exhibited a 1.7 fold increase in AUC in comparison to patients that received plerixafor alone.

TABLE 8
Peak CD34+ cell levels in response to plerixafor alone versus
simultaneous or staggered MGTA-145 + plerixafor

| MGTA-145 dose | | Peak CD34+/μL | CD34+ AUC_{2-8 h} | Tmax* (h) |
|---|---|---|---|---|
| (mg/kg) | Regimen | Median (range) | Median (range) | Median (range) |
| 0 | Plerixafor | 20 (13-65) | 102 (61-341) | 8 (3-12) |
| | alone | | | |
| 0.03 | Simultaneous | 26 (20-70) | 130 (96-327) | 6 (4-6) |
| 0.075 | MGTA-145 + | 27 (13-67) | 136 (67-353) | 7 (6-12) |
| 0.15 | plerixafor | 29 (9-55) | 137 (46-252) | 6 (6-8) |
| All doses | | 27 (9-70) | 132 (46-353) | 6 (4-12) |
| 0.03 | Staggered | 36 (13-63) | 168 (66-316) | 6 (3-10) |
| 0.07 | MGTA-145 + | 30 (11-55) | 140 (48-254) | 6 (3-10) |
| 0.075 | plerixafor | 46 (40-52) | 221 (208-233) | 7 (6-8) |
| All doses | | 34 (11-63) | 168 (48-316) | 6 (3-10) |
| All doses: fold | Staggered | 1.8x | 1.7x | N/A |
| change versus | MGTA-145 + | | | |
| plerixafor alone | plerixafor | | | |

TABLE 9 shows the breakdown of CD34+ mobilization across patients that received either simultaneous or staggered administration of MGTA-145+plerixafor. Specifically, the CD34+ levels for each patient were analyzed to determine the percentage of patients in each dose group to achieve ≥20 and/or ≥40 CD34+ cells/μL at peak. Generally, a higher percentage of patients that received staggered plerixafor and MGTA-145 (any of 0.03 mg/kg, 0.07 mg/kg, or 0.075 mg/kg) achieved a peak CD34+ count of ≥20 or ≥40 per μL in comparison to patients that received plerixafor alone or simultaneous plerixafor and MGTA-145. In particular, of the 22 patients receiving staggered plerixafor and MGTA-145 (any of 0.03 mg/kg, 0.07 mg/kg, or 0.075 mg/kg), 18 (83%) exhibited a peak of at least 20 CD34+ cells/μL in response to treatment. This represents a 1.7 fold increase in comparison to patients that received plerixafor alone. Similarly, of the 22 patients receiving staggered plerixafor and MGTA-145 (any of 0.03 mg/kg, 0.07 mg/kg, or 0.075 mg/kg), 9 exhibited a peak of at least 40 CD34+ cells/μL in response to treatment. This represents a 2.9 fold increase in comparison to patients that received plerixafor alone.

TABLE 9
Categorized patients according to their peak
CD34+ cell levels in response to plerixafor
alone versus or staggered + plerixafor

| MGTA-145 | | | |
|---|---|---|---|
| dose (mg/kg) | Regimen | ≥20 (%) | ≥40 (%) |
| 0 | Plerixafor alone | 50% (7/14) | 14% (2/14) |
| 0.03 | Simultaneous | 83% (5/6) | 17% (1/6) |
| 0.075 | MGTA-145 + | 83% (5/6) | 17% (1/6) |
| 0.15 | plerixafor | 83% (5/6) | 17% (1/6) |
| All doses | | 83% (15/18) | 17% (3/18) |
| 0.03 | Staggered | 80% (8/10) | 40% (4/10) |
| 0.07 | MGTA-145 + | 80% (8/10) | 30% (3/10) |
| 0.075 | plerixafor | 100% (2/2) | 100% (2/2) |
| All doses | | 82% (18/22) | 41% (9/22) |
| | Historical Plerixafor | 28% (7/25) | 4% (1/25) |
| | alone data¹ | | |

¹Devine et al., Blood. 2008.

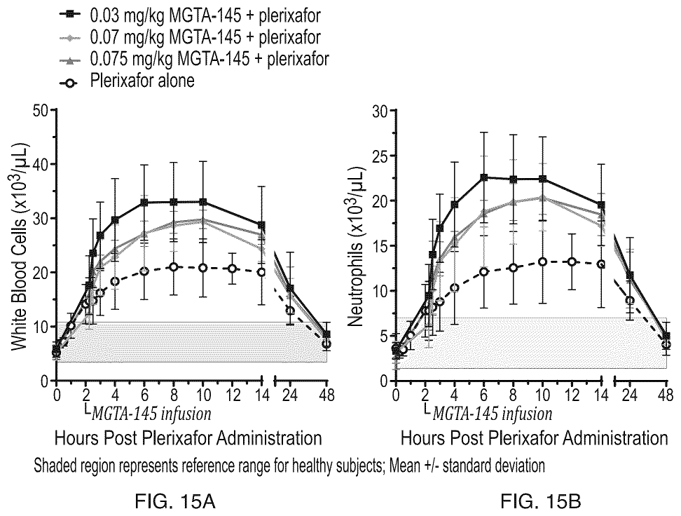

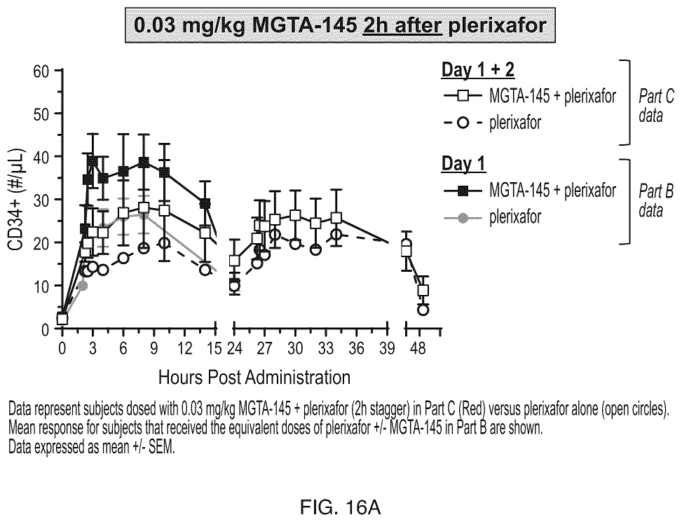

FIGS. 15A and 15B show the mobilization of WBCs and neutrophils, respectively, over the course of 24 hours following staggered combination treatment of MGTA-145 and plerixafor. As shown in FIG. 15A, the staggered combination treatment of 0.03 mg/kg MGTA-145 and plerixafor generally induced significantly higher WBC mobilization than the staggered combination treatment of 0.70/0.075 mg/kg MGTA-145+plerixafor or plerixafor alone. More specifically, the staggered administration of plerixafor followed by 0.03 mg/kg MGTA-145 induced a peak mobilization of WBC of ˜35×10³ cells/μL at a timepoint of 6 hours post plerixafor administration. The WBC mobilization remained steady for the next 2 hours, achieving ˜34×10³ cells/μL at a timepoint of 8 hours post plerixafor administration. Even at 24 hours post plerixafor administration, the WBC mobilization level (˜15×10³ cells/μL) remained higher than the initial WBC level prior to treatment (˜6×10³ cells/μL). In contrast, the staggered administration of plerixafor followed by 0.070/0.075 mg/kg MGTA-145 induced a peak mobilization of WBC of ˜21×10³ cells/μL at a timepoint of 8 hours

As shown in FIG. 15B, the staggered combination treatment of 0.03 mg/kg MGTA-145 and plerixafor generally induced significantly higher neutrophil mobilization than the staggered combination treatment of 0.70/0.075 mg/kg MGTA-145+plerixafor or plerixafor alone. More specifically, the staggered administration of plerixafor followed by 0.03 mg/kg MGTA-145 induced a peak mobilization of neutrophils of ˜25×10³ cells/μL at a timepoint of 6 hours post plerixafor administration. The neutrophil mobilization remained steady for the next 2 hours, achieving ˜24×10³ cells/μL at a timepoint of 8 hours post plerixafor administration. Even at 24 hours post plerixafor administration, the neutrophil mobilization level (˜12×10³ cells/μL) remained higher than the initial neutrophil level prior to treatment (˜3×10³ cells/μL). In contrast, the staggered administration of plerixafor followed by 0.070/0.075 mg/kg MGTA-145 induced a peak neutrophil mobilization of ˜17×10³ cells/μL at a timepoint of 8 hours.

### Example 5: Staggered Combination Therapy of MGTA-145 and Plerixafor on Two Consecutive Days

Part C of the Phase 1 trial of Example 1 involved administering a combination dose of MGTA-145 or placebo and plerixafor on two consecutive days. The dose of MGTA-145 was one of 0.03 mg/kg, 0.075 mg/kg, or 0.15 mg/kg. The dose of plerixafor was 240 μg/kg (0.24 mg/kg). The dose of MGTA-145 was administered 2 hours after plerixafor (i.e., staggered administration). Cells were collected on the second day.

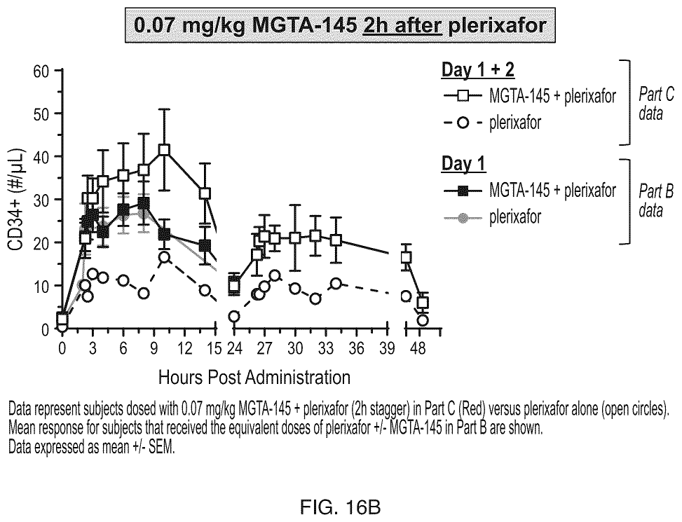

As shown in FIG. 16A, administration of 0.03 mg/kg MGTA-145 and plerixafor on two consecutive days led to the mobilization of similar numbers of CD34+ cells on the second day (compare hours 0-15 to hours 24-48 on the day 1+2 graph (Part C data). These results show that administration of MGTA-145 and plerixafor and cell collection on two consecutive days can be performed for patients with low CD34+ yields in order to mobilize sufficient numbers of CD34+ cells. Similar results were found for administration of 0.07 mg/kg MGTA-145 and plerixafor (FIG. 16B).

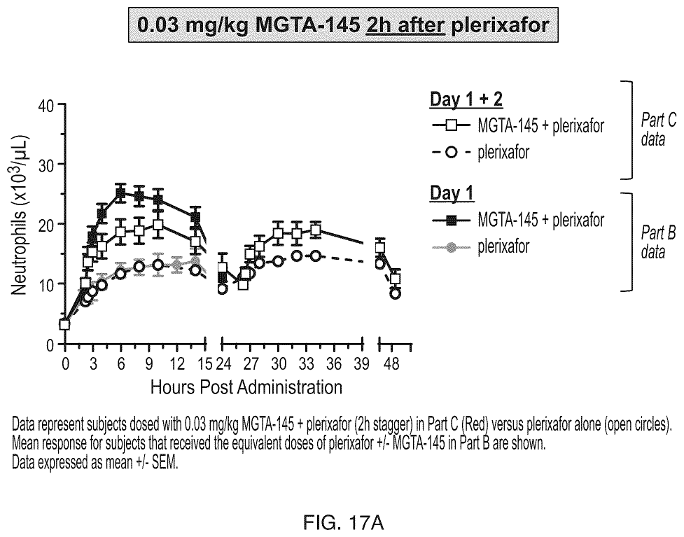

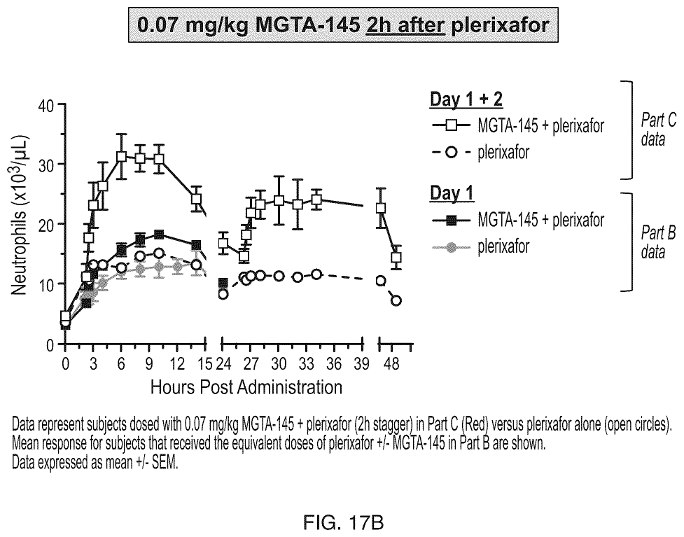

As shown in FIG. 17A, administration of 0.03 mg/kg MGTA-145 and plerixafor on two consecutive days led to the release of neutrophils on both days, with similar numbers of neutrophils released on the second day. Similar results were found for administration of 0.07 mg/kg MGTA-145 and plerixafor (FIG. 17B).

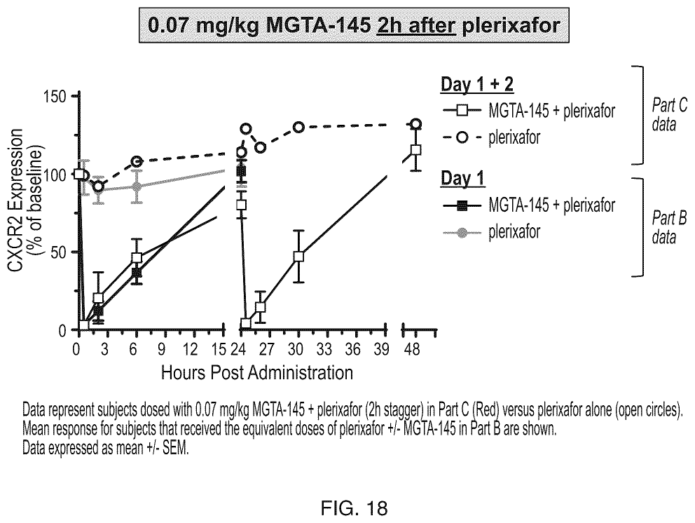

Without wishing to be bound by theory, it is believed that fewer cells are mobilized on day 2 because CXCR2 expression becomes downregulated following the initial administration of MGTA-145. FIG. 18 provides a graph showing that CXCR2 expression recovers to ˜80% of baseline prior to the second dose of MGTA-145 (0.07 mg/kg, staggered dosing).

A summary of CD34+ levels for one day/first day treatment is shown in TABLES 10A and 10B. TABLE 10B shows peak CD34+ cell levels in response to MGTA-145 alone across 5 different MGTA-145 doses (0.03 mg/kg, 0.075 mg/kg, 0.15 mg/kg, 0.3 mg/kg and 0.015 mg/kg, and 0.0075 mg/kg) administered to patients enrolled in Part A. TABLES 10A and 10B provide peak CD34+ cell levels in response to plerixafor alone or a MGTA-145+plerixafor treatment across three different MGTA-145 doses (0.015 mg/kg, 0.03 mg/kg and 0.075 mg/kg (or 0.070 mg/kg)) administered to patients enrolled in Part B. During Part B, two patients were dosed with 0.075 mg/kg, and then the remaining 6 patients were dosed down at 0.07 mg/kg. These data are pooled in TABLE 10A and shown separately in TABLE 10B. TABLES 10A and 10B also provide peak CD34+ cell levels in response to plerixafor alone or a MGTA-145+plerixafor treatment across two different MGTA-145 doses (0.03 mg/kg and 0.075 mg/kg) administered to patients enrolled in Part C of the Phase 1 clinical trial. For Part C patients, only the first day data is included in the table. In addition to the peak CD34+ levels, the CD34+ levels for each patient in Parts B and C were analyzed to determine the percentage of patients in each dose group to achieve ≥20 and/or ≥40 CD34+ cells/μL at peak, and these data are also provided in TABLES 10A and 10B. Generally, a higher percentage of patients that received the combination of plerixafor and MGTA-145 (any of 0.03 mg/kg or 0.075 mg/kg) achieved a peak CD34+ count of ≥20 or ≥40 cells/μL in comparison to patients that received plerixafor alone. In particular, for 20 of the patients in Part B who received combination plerixafor and MGTA-145 (0.015 mg/kg, 0.03 mg/kg, or 0.075 mg/kg/0.070 mg/kg), 17 patients achieved a CD34+ count of at least 20 cells/μL at peak. This represents a 1.3 fold increase in comparison to patients that received plerixafor alone. Additionally, 8 of the 20 patients achieved a CD34+ count of at least 40 cells/μL at peak. This represents a 1.9 fold increase in comparison to patients that received plerixafor alone. For the 8 patients in Part C who received the combination plerixafor and MGTA-145 (any of 0.03 mg/kg or 0.075 mg/kg), 6 achieved a peak CD34+ count of at least 20 cells per microliter whereas none of the patients in Part C who received plerixafor alone achieved a peak CD34+ count of at least 20 cells per microliter. Additionally, 3 of the 8 patients achieved a peak CD34+ count of at least 40 cells per microliter whereas none of the patients in Part C who received plerixafor alone achieved a peak CD34+ count of at least 40 cells per microliter.

TABLE 10A
Summary of One Day Mobilization Data

| | | MGTA-145 dose | Peak CD34+ (#/μL) | | | | |
|---|---|---|---|---|---|---|---|
| Part | Regimen | (mg/kg) | Median (range) | % ≥20/μL | | % ≥40/μL | |
| B | Plerixafor | 0 | 26 (13-78) | 64% | (9/16) | 21% | (3/16) |
| | Plerixafor + | 0.015 | 35 (17-78) | 83% | (5/6) | 33% | (2/6) |
| | MGTA-145 | 0.03 | 40 (18-63) | 83% | (5/6) | 50% | (3/6) |
| | | 0.075 | 30 (11-52) | 88% | (7/8) | 38% | (3/8) |
| C | Plerixafor | 0 | 18 (16-20) | 0% | (0/2) | 0% | (0/2) |
| | Plerixafor + | 0.03 | 31 (14-53) | 75% | (3/4) | 25% | (1/4) |
| | MGTA-145 | 0.075 | 40 (22-65) | 75% | (3/4) | 50% | (2/4) |
| Pooled | Plerixafor | 0 | 20 (13-78) | 50% | (7/14) | 14% | (2/14) |
| B + C | Plerixafor + | 0.03 | 36 (14-63) | 80% | (8/10) | 40% | (4/10) |
| | MGTA-145 | 0.075 | 33 (11-65) | 92% | (11/12) | 42% | (5/12) |

TABLE 10B
Summary of CD34+ Mobilization- Study 145-HV-101 Parts A Through C

| | | MGTA-145 dose | No. of | Peak CD34+ (#/μL) | | Percent ≥20/μL | |
|---|---|---|---|---|---|---|---|
| Part | Regimen | (mg/kg) | Subjects | Median (range) | | % (n) | |
| A | MGTA-145 | 0.03 | 4 | 12 | (2-19) | 0 | (0/4) |
| | on Day 1 | 0.075 | 4 | 4 | (3-9) | 0 | (0/4) |
| | | 0.15 | 4 | 4 | (3-5) | 0 | (0/4) |
| | | 0.3 | 4 | 3 | (3-13) | 0 | (0/4) |
| | | 0.015 | 4 | 5 | (4-8) | 0 | (0/4) |
| | | 0.0075 | 4 | 5 | (2-12) | 0 | (0/4) |
| | Pooled | 0 | 12 | 2 | (1-4) | 0 | (0/12) |
| | placebo | | | | | | |
| B | | 0.03 | 6 | 26 | (20-70) | 83 | (5/6) |
| | MGTA-145 | 0.075 | 6 | 29 | (13-67) | 83 | (5/6) |
| | administered | 0.15 | 6 | 29 | (9-55) | 83 | (5/6) |
| | 10 min | | | | | | |
| | following | | | | | | |
| | plerixafor on | | | | | | |
| | Day 1 | | | | | | |
| | MGTA-145 | 0.075 | 2 | 46 | (40-52) | 100 | (2/2) |
| | administered 2 | 0.07 | 6 | 26 | (11-42) | 83 | (5/6) |
| | hr following | 0.03 | 6 | 40 | (18-63) | 83 | (5/6) |
| | plerixafor on | | | | | | |
| | Day 1 | 0.015 | 6 | 36 | (18-86) | 83 | (5/6) |
| | Pooled | 0 | 14 | 26 | (13-78) | 64 | (9/14) |
| | placebo + | | | | | | |
| | plerixafor on | | | | | | |
| | Day 1 | | | | | | |
| C | MGTA-145 | 0.07 | 4 | 40 | (22-65) | 100 | (4/4) |
| | administered 2 | | | | | | |
| | hr following | 0.03 | 4 | 31 | (14-53) | 75 | (3/4) |
| | plerixafor on | | | | | | |
| | Day 1 | | | | | | |
| | MGTA-145 | 0.07 | 4 | 28 | (13-40) | 75 | (3/4) |
| | administered 2 | | | | | | |
| | hr following | 0.03 | 4 | 23 | (16-36) | 75 | (3/4) |
| | plerixafor on | | | | | | |
| | Day 2 | | | | | | |
| | Pooled | 0 | 2 | 18 | (16-20) | 0 | (0/2) |
| | placebo + | | | | | | |
| | plerixafor on | | | | | | |
| | Day 1 | | | | | | |
| | Pooled | 0 | 2 | 17 | (12-22) | 50 | (1/2) |
| | placebo + | | | | | | |
| | plerixafor on | | | | | | |
| | Day 2 | | | | | | |
| Pooled | MGTA-145 | 0.07 | 10 | 30 | (11-65) | 80 | (8/10) |
| B + C | administered 2 | 0.03 | 10 | 36 | (14-63) | 80 | (8/10) |
| | hr following | 0.015 | 6 | 36 | (18-86) | 83 | (5/6) |
| | plerixafor on | | | | | | |
| | Day 1 | | | | | | |
| | Pooled | 0 | 16 | 23 | (13-78) | 56 | (9/16) |
| | placebo + | | | | | | |
| | plerixafor on | | | | | | |
| | Day 1 | | | | | | |

TABLE 11
Comparison with G-CSF

| | G-CSF | MGTA-145 + plerixafor |
|---|---|---|
| Mechanism of Action | Bone remodeling | Chemokine cell migration |
| Time to mobilize and collect | 5+ days | <1 day |
| Tolerability | Majority with bone pain, | Majority with transient, |
| | headache, myalgia, and/or | grade 1 back pain (most <20 |
| | fatigue (up to 1+ week)^{a} | minutes) |
| Efficacy (≥2 × 10⁶ CD34⁺/kg) | 78%^{b} | 88% (7/8) |
| Quality of CD34⁺ (% CD90⁺) | 6.9% | 33% |
| Function of CD34⁺ | — | >10x increased engraftment |

^{a}Pulsiper et al. (2009) Blood 113(15): 3604-3611.
^{b}Holig (2013) Transfus Med Hemother 40: 225-235.

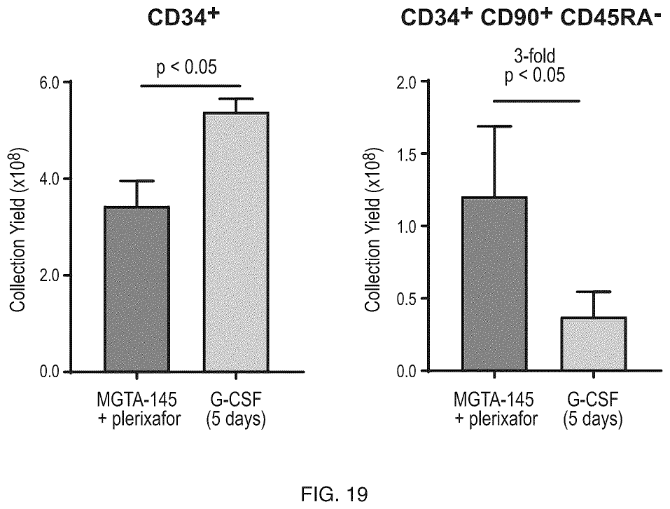

TABLE 11 shows a comparison of the features of MGTA-145+plerixafor-based mobilization and G-CSF-based mobilization. As compared to G-CSF, which causes mobilization via bone remodeling, MGTA-145+plerixafor stimulate chemokine cell migration. MGTA-145+plerixafor requires much less time for mobilization and collection (less than 1 day, vs. 5+ days for G-CSF). MGTA-145+plerixafor is more tolerable, with subjects showing fewer side effects as referenced in TABLE 11. Further, MGTA-145+plerixafor is more efficacious, with 88% of subjects tested mobilizing ≥2×10⁶ CD34+ cells/kg (as compared with 78% of subjects administered G-CSF, reported by Holig, supra), and the quality of cells is superior, with 35% of CD34+ cells being CD90+, compared with only 10% for G-CSF. The superiority of cells mobilized using MGTA-145+plerixafor is also demonstrated in FIG. 19, which shows that MGTA-145+plerixafor mobilizes 3-fold higher numbers of CD90+ cells than does G-CSF.

In addition, as described in Example 7 below, MGTA-145+plerixafor-mobilized cells provide 5× increased engraftment in mice.

### Example 6: Apheresis Product Obtained from Patients Receiving MGTA-145 and Plerixafor Combination Therapy

TABLE 12A shows CD34+ and CD34+CD90+CD45RA− cell yields from four subjects (labeled as subjects 801, 807, 817, and 821) who underwent apheresis procedures following treatment with a combination of 0.03 mg/kg MGTA-145 and plerixafor (staggered 2 hour protocol). The table documents the body weight of each subject, the total CD34+ cells that were obtained through the apheresis procedure, the normalized CD34+ cells/kg of body weight, and the percentage of CD34+ cells that were CD90+CD45RA−. Of note, one of the subjects (subject 821) completed only 13 L (65%) of the planned 20 L of apheresis, thereby explaining the lower overall CD34+ cell yield. The total CD34+ cells that were obtained for each patient through the apheresis procedure ranged from 239×10⁶ cells up to 500×10⁶ cells. The normalized CD34+ cells/kg of body weight ranged from 2.7×10⁶ cells/kg up to 5.3×10⁶ cells/kg, with a median of 4.3×10⁶ cells/kg, higher than the clinical threshold for transplant. The percentage of CD34+CD90+CD45− cells amongst the total CD34+ cell population ranged from 19% to 41%.

TABLE 12A
CD34+ cell yield from four patients undergoing apheresis
treatment following treatment with a combination of 0.03
mg/kg MGTA-145 and plerixafor (staggered 2 hour protocol)

| | Body weight | Total CD34⁺ Yield | CD34⁺/kg | CD90⁺ | CD90⁺ |
|---|---|---|---|---|---|
| Subject | (kg) | (×10⁶ cells) | (×10⁶ cells) | (×10⁸ cells) | (%)^{b} |
| 801 | 78.3 | 319 | 4.1 | 1.24 | 39% |
| 807 | 72.6 | 322 | 4.4 | 1.32 | 41% |
| 817 | 94.2 | 500 | 5.3 | 1.34 | 27% |
| 821^{a} | 89.6 | 239 | 2.7 | 0.46 | 19% |
| Median | 84 | 321 | 4.3 | 1.3 | 33% |

| | |
|---|---|
| Clinical Threshold for Transplant | ≥2.0 |

^{a}Subject 821 completed 13 L (65%) of the planned 20 L apheresis
^{b}CD90+ (%) represents the percentage of collected CD34+ cells that were CD90+ CD45RA−

TABLE 12B shows CD34+ and CD34+CD90+CD45RA− cell yields from four subjects (labeled as subjects 837, 838, 847, and 850) who underwent apheresis procedures following treatment with combination of 0.015 mg/kg MGTA-145 and plerixafor (staggered 2 hour protocol). The table documents the total CD34+ cells that were obtained through the apheresis procedure, the normalized CD34+ cells/kg of body weight, and the percentage of CD34+ cells that were CD90+CD45RA−. The total CD34+ cells that were obtained for each patient through the apheresis procedure ranged from 118×10⁶ cells up to 525×10⁶ cells. The normalized CD34+ cells/kg of body weight ranged from 1.5×10⁶ cells/kg up to 7.0×10⁶ cells/kg, with a median of 3.7×10⁶ cells/kg, higher than the clinical threshold for transplant. The percentage of CD34+CD90+CD45− cells amongst the total CD34+ cell population ranged from 28% to 41%.

TABLE 12B
CD34+ cell yield from four patients undergoing apheresis
treatment following treatment with a combination of 0.015
mg/kg MGTA-145 and plerixafor (staggered 2 hour protocol)

| | Total CD34⁺ | | | |
|---|---|---|---|---|
| | Yield | CD34⁺/kg | CD90⁺ | CD90⁺ |
| Subject | (×10⁶ cells) | (×10⁶ cells) | (×10⁸ cells) | (%)^{a} |
| 837 | 525 | 7.0 | 2.14 | 41% |
| 838 | 274 | 3.4 | 1.01 | 37% |
| 847 | 345 | 3.9 | 0.982 | 28% |
| 850 | 118 | 1.5 | 0.398 | 34% |
| Median | 310 | 3.7 | 1.0 | 35% |

| | |
|---|---|
| Clinical Threshold for Transplant | ≥2.0 |

^{a}CD90+ (%) represents the percentage of collected CD34+ cells that were CD90+ CD45RA−

TABLE 12C shows CD34+ and CD34+CD90+CD45RA− cell yields from three subjects (labeled as subjects G-CSF837, 838, 847, and 850) who underwent apheresis procedures following treatment with combination of 0.015 mg/kg MGTA-145 and plerixafor (staggered 2 hour protocol). The table documents the body weight of each subject, the total CD34+ cells that were obtained through the apheresis procedure, the normalized CD34+ cells/kg of body weight, and the percentage of CD34+ cells that were CD90+CD45RA−. The total CD34+ cells that were obtained for each patient through the apheresis procedure ranged from 118×10⁶ cells up to 525×10⁶ cells. The normalized CD34+ cells/kg of body weight ranged from 1.5×10⁶ cells/kg up to 7.0×10⁶ cells/kg, with a median of 3.7×10⁶ cells/kg, higher than the clinical threshold for transplant. The percentage of CD34+CD90+CD45-cells amongst the total CD34+ cell population ranged from 28% to 41%.

TABLE 13 provides apheresis collection yield at the 0.015 mg/kg dose and 0.03 mg/kg dose. At the 0.015 mg/kg dose, a mean of 4.0 CD34+ (×10⁶) cells and median of 3.7 CD34+ (×10⁶) cells were collected, with a mean of 1.4 CD90+ (×10⁶) cells and median of 1.2 CD90+ (×10⁶) cells. Accordingly, at the 0.015 mg/kg dose, 37% of CD34⁺ cells collected were CD90+. At the 0.03 mg/kg dose, a mean of 4.1 CD34+ (×10⁶) cells and median of 4.3 CD34+ (×10⁶) cells were collected, with a mean of 1.3 CD90+ (×10⁶) cells and median of 1.5 CD90+ (×10⁶) cells. Accordingly, at the 0.015 mg/kg dose, 31% of CD34⁺ cells collected were CD90+.

TABLE 13
Apheresis Collection at 0.015 versus 0.03 mg/kg dose
of MGTA-145, 2 h stagger

| MGTA- | Total CD34⁺ | | | | | | | |
|---|---|---|---|---|---|---|---|---|
| 145 | Yield (x10⁶) | | | | | | | CD90⁺ |
| dose | Median | CD34⁺/kg (x10⁶) | | | CD90⁺/kg (x10⁶)^{a} | | | (% of |
| (mg/kg) | (range) | Mean | Median | Range | Mean | Median | Range | CD34⁺) |
| 0.015 | 310 (118-525) | 4.0 | 3.7 | 1.5-7.0 | 1.4 | 1.2 | 0.5-2.8 | 37% |
| 0.03 | 321 (239-500) | 4.1 | 4.3 | 2.7-5.3 | 1.3 | 1.5 | 0.5-1.8 | 31% |

Collection data reflects internal analysis.
ªCD90+ cells defined as CD34⁺ CD90⁺ CD45RA⁻ cells.
6.9% of G-CSF-mobilized CD34⁺ cells are CD90⁺ CD45RA⁻ cells (internal data, n = 3).

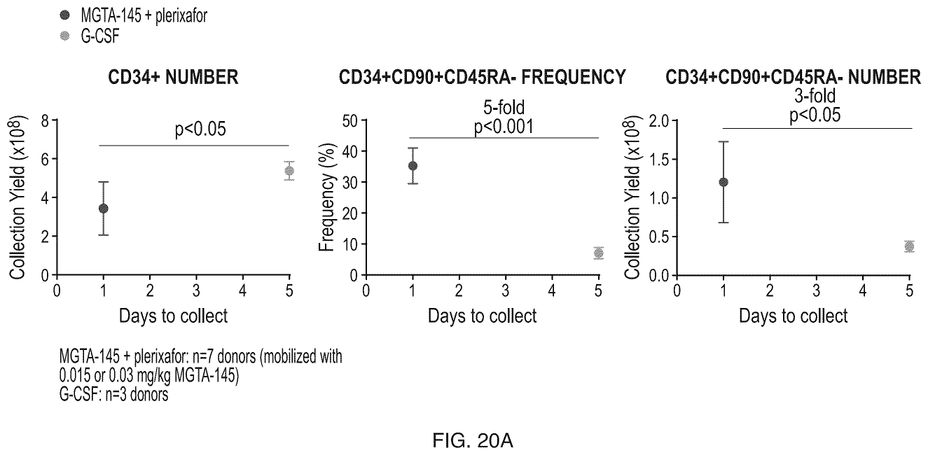

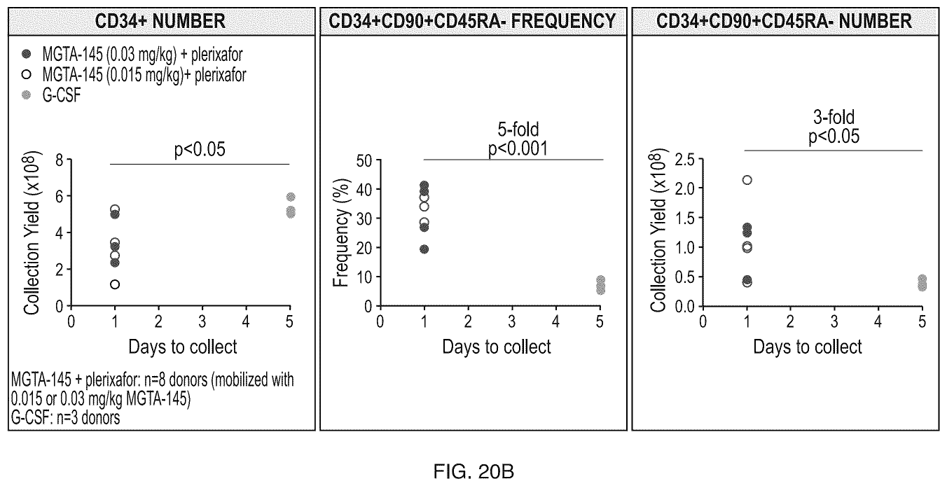

A summary of these findings is provided in FIG. 20A and FIG. 20B, which shows, from left to right, the collection yield of CD34+ cells following mobilization by MGTA-145+plerixafor or G-CSF, the frequency of CD34+CD90+CD45RA+ cells following mobilization by MGTA-145+plerixafor or G-CSF, and collection yield of CD34+CD90+CD45RA+ cells following mobilization by MGTA-145+plerixafor or G-CSF.

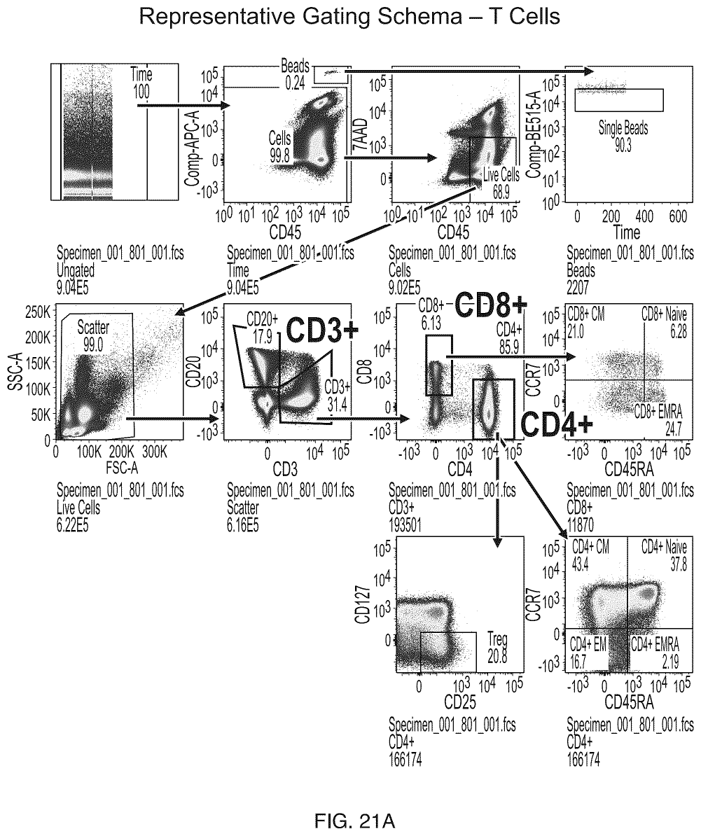

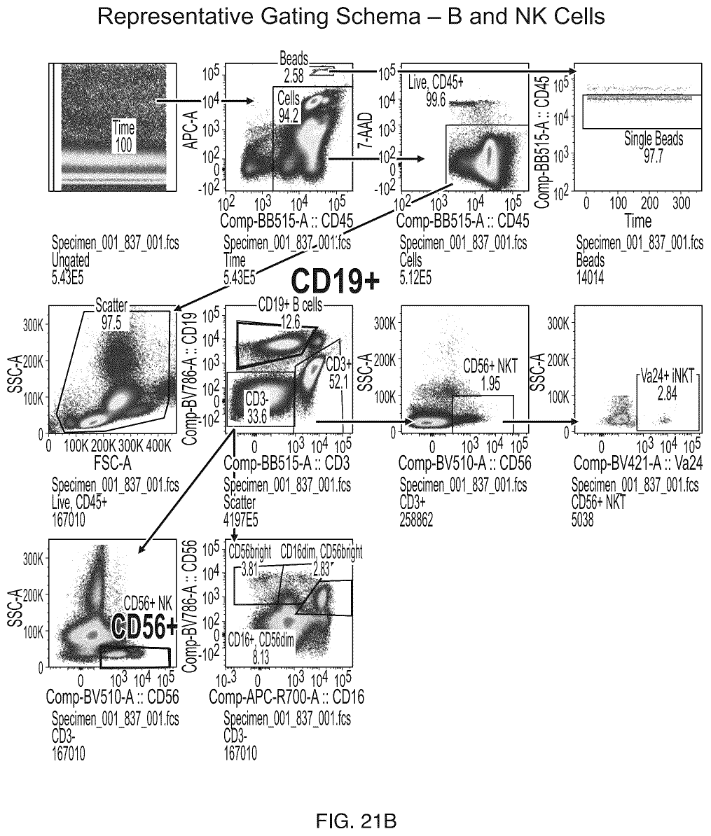

Flow cytometry was additionally performed to identify yield of different populations of cells (e.g., B, T, and NK cells) following mobilization by MGTA-145+plerixafor. Representative flow plots for mice transplanted with MGTA-145+plerixafor-mobilized CD34+ cells are shown in FIGS. 21A and 21B. Specifically, FIG. 21A depicts a representative gating scheme for quantifying T cells (CD3+, CD4+, CD8+). FIG. 21B depicts a representative gating scheme for quantifying B (CD19+) and NK cells (CD56+). TABLE 14 documents the median concentrations of CD3+ T cells, CD4+ T cells, CD8+ T cells, CD19+ B cells, and CD56+NK cells across different mobilization regimes (e.g., Part D MGTA-145+plerixafor, plerixafor alone, or G-CSF alone as documented in literature e.g., Devine et al 2008 and Chen et al., 2019). TABLE 15 documents the total nucleated cell (TNC) yield as a result of the MGTA-145+plerixafor mobilization regime in comparison to G-CSF alone as previously reported by Singhal et al, BMT, 2000. TABLE 16 documents the frequencies of graft cell subsets (e.g., T cells, B cells, and NK cells).

In particular, while CD3+ T-cell numbers were comparable between MGTA-145+plerixafor and plerixafor alone, MGTA-145+plerixafor mobilized 0.2 (0.0-0.6)×10⁸/kg CD8+ T-cells, constituting 1.8 (0.5-4.8)% of the graft, a number and proportion significantly lower than that mobilized by either G-CSF or plerixafor alone. As higher numbers CD8+ T cells are associated with higher rates of GvHD, having lower numbers of CD8+ T cells in the MGTA-145+plerixafor graft may be beneficial.

TABLE 14
Comparison in graft composition between mobilization regimens

| | Part D | | | | |
|---|---|---|---|---|---|
| | MGTA-145 + | Devine et al, 2008^{b} | | Chen et al., 2019^{c} | |
| | plerixafor | Plerixafor | G-CSF | Plerixafor | |
| | N = 7 | N = 24 | N = 8 | N = 50+ | G-CSF |
| | Median | Median | Median | Median | N = 112+ |
| | (range) | (range) | (range) | (range) | Median |
| CD34+ | 4.1 (1.5-7.0) | 2.9 (1.2-6.3) | 4.2 (2.5-18.7) | 4.7 (0.9-9.6) | 4.2 |
| (×10⁶/kg) | | | | | |
| CD3+ | 4.0 (3.3-6.2) | 4.6 (1.5-7.8) | 1.3 (1.2-6.8) | 6.0 (2.7-13.0) | 2.5 |
| (×10⁸/kg) | | | | | |
| CD4+ | 3.7 (3.0-5.0) | 3.2 (1-5.7) | 1.1 (0.7-3.2) | 3.6 (2.0-9.4) | 1.5 |
| (×10⁸/kg) | | | | | |
| CD8+ | 0.2 (0.0-0.6) | 1.3 (0.4-3.4) | 0.4 (0.3-3.4) | 2.2 (0.5-5.0) | 0.7 |
| (×10⁸/kg) | | | | | |
| CD19+ | 1.8 (1.1-1.9) | 1.0 (0.2-2.4) | — | 1.5 (0.2-7.4) | 0.5 |
| (×10⁸/kg) | | | | | |
| CD56+ | 0.5 (0.2-1.0) | 0.3 (0.1-1.0) | 0.2 (0.1-0.5) | 0.4 (0.0-1.1) | 0.1 |
| (×10⁸/kg) | | | | | |

^{a} Data are pooled for donors mobilized with 0.03 or 0.015 mg/kg MGTA-145 + plerixafor. Donor that only completed 13 L of planned 20 L collection excluded from the analysis.
^{b}Devine dataset reflects median yield of 1 LP for plerixafor and G-CSF grafts.
^{c}Chen dataset reflects median yield of 2 LP for plerixafor and 1 LP for G-CSF grafts.

TABLE 15
Total nucleated cell (TNC) yield, (×10⁸ cells)

| | MGTA-145 + plerixafor | G-CSF |
|---|---|---|
| | N = 7 | N = 40^{a} |
| | Median (range) | Median (range) |
| TNC | 855 (667-1058) | — |
| TNC/kg donor weight | 9.7 (8.8-12.7) | 7.2 (3.2-14.0) |

^{a}Data represent 40 healthy donors mobilized with G-CSF as described previously (Singhal et al, BMT. 2000).

TABLE 16
Frequencies of graft cell subsets, (%)^{b}

| | MGTA-145 + plerixafor | | G-CSF | |
|---|---|---|---|---|
| | N = 4 | | N = 78^{c} | |
| | Median (range) | | Median (range) | |
| CD3+ T cells | 40.8 | (31.7-51.1) | 45.3 | (21.8-75.4) |
| CD4+ T cells | 32.9 | (27.2-48.1) | 29.8 | (12.6-56.5) |
| CD8+ T cells | 1.8 | (0.5-4.8) | 11.8 | (4.1-29.2) |
| Tregs | 2.6 | (0.7-5.5) | 3.5 | (0.9-8.2) |
| CD19+ B cells | 14.7 | (12.3-19.7) | 10.5 | (0.9-34.8) |
| CD56+ NK cells | 5.0 | (2.1-8.3) | 3.2 | (0.5-18.4) |
| NKT cells | 1.6 | (0.6-2.7) | — | |
| iNKT cells | 0.01 | (0.00-0.03) | 0.04 | (0.00-1.07) |

^{b}Frequencies reflect % of CD45+ except for NKT/iNKT cells (% of CD3+) and Tregs (% of CD4+ T cells).
^{c}Data represent 78 healthy donors mobilized with G-CSF as described previously (Chaidos et al, Blood. 2012).

### Example 7: MGTA-145+Plerixafor Mobilizes an Immunosuppressive Graft Containing Large Numbers of Hematopoietic Stem Cells Capable of Robust Engraftment

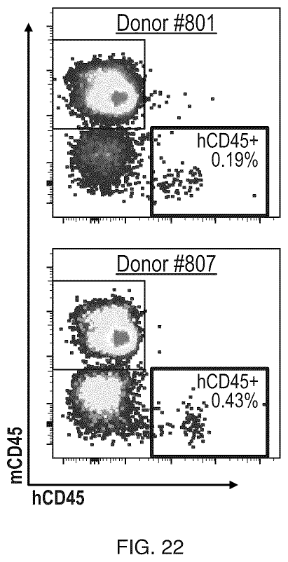

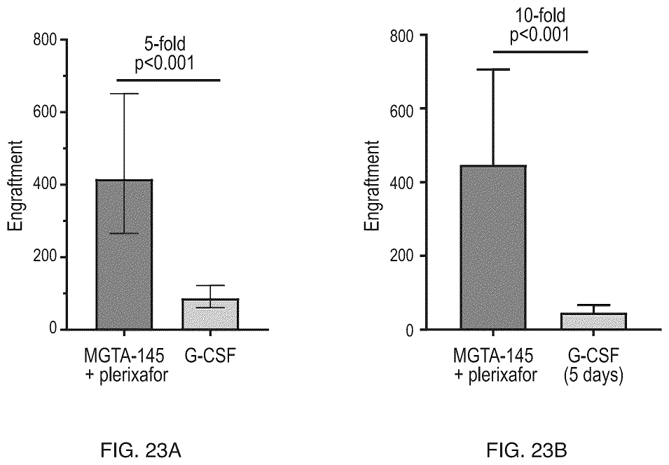

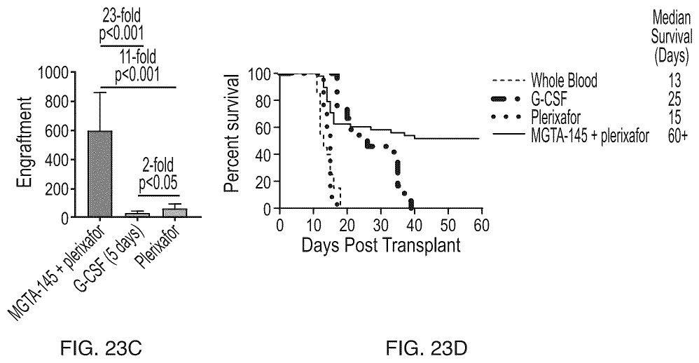

CD34+ cells from MGTA-145+plerixafor-mobilized donors (Part D, n=2 donors at 0.03 mg/kg dose level) or G-CSF-mobilized peripheral blood (n=3) were transplanted into sublethally irradiated (200 cGy) NSG mice at limit dilution (3 cell doses per donor). Engraftment of human CD45+ (hCD45+ frequency) and lineage composition (CD3, CD19, CD33) in peripheral blood was measured at week 4 and week 12 post-transplant by flow cytometry. Representative flow plots for mice transplanted with MGTA-145+plerixafor-mobilized CD34+ cells are shown in FIG. 22. SCID-repopulating cell (SRC) number per 1×10⁶ cells and statistical significance was determined by ELDA at week 4, week 12, and week 16 (n-3-4 donors, n-7-8 mice per group). As shown in FIGS. 23A-C, cells mobilized by MGTA-145 and plerixafor showed a 5-fold increase in engraftment at week 4 post-transplant, a 10-fold increase in engraftment at week 12 post-transplant, and a 23-fold increase in engraftment at week 16 post-transplant as compared to cells mobilized by G-CSF. Cells mobilized by MGTA-145 and plerixafor showed an 11-fold increase in engraftment at week 16 post-transplant as compared to cells mobilized by plerixafor alone (FIG. 23C). Data are expressed as SRC number+/−95% CI. To determine the effect of the mobilization regimen on xenogeneic GvHD, a xenograft GvHD model in NSG mice was developed where 6×10⁶ PBMCs from various graft sources were infused into sublethally-irradiated animals (n=3-6 donors per graft source). MGTA-145+plerixafor mobilized grafts resulted in less GvHD than G-CSF (p<0.01) or plerixafor (p<0.001) grafts (FIG. 23D). In vivo cellular subset depletion studies suggested that the GvHD protective effect in MGTA-145+plerixafor grafts may be in part due to immunosuppressive monocytes which were not present, or present to a lesser degree, in grafts from donors mobilized with G-CSF or plerixafor.

This example demonstrates that MGTA-145+plerixafor is a rapid, reliable, and G-CSF free method to obtain high numbers of HSCs with durable engraftment potential and a graft with immunosuppressive properties. The example suggests that MGTA-145+plerixafor is an effective single-day mobilization/collection regimen for both autologous and allogeneic stem cell transplantation resulting in enhanced engraftment and reduced GvHD.

### Example 8: MGTA-145+Plerixafor Mobilizes Higher Numbers of HSCs Mice Relative to G-CSF

The following examples demonstrates that MGTA-145+plerixafor mobilizes higher numbers of HSCs with durable primary and secondary engraftment in mice relative to other mobilization regimens.

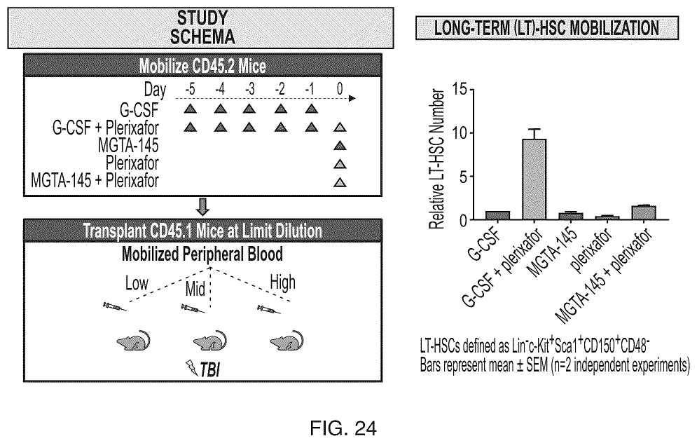

Mice (CD45.1) were mobilized according to the scheme shown in FIG. 24 and mobilized cells transplanted into CD45.2 mice at limit dilution. MGTA-145+plerixafor mobilized higher numbers of LT-HSC cells (Lin⁻c-Kit⁺Sca1⁺CD150⁺CD48⁻) than did the standard of care, G-CSF, while G-CSF+plerixafor mobilized the highest numbers of LT-HSC cells.

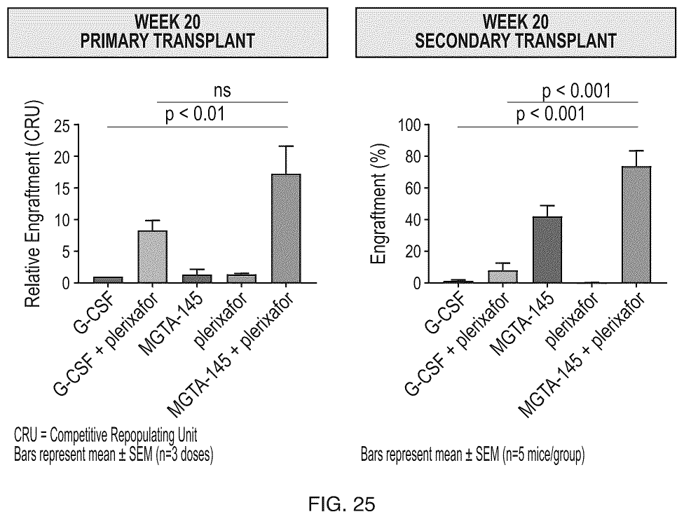

As shown in FIG. 25, left panel, primary transplantation of cells mobilized with MGTA-145+plerixafor led to higher relative engraftment (CRU) compared to any of the other mobilizing regimens, suggesting that MGTA-145+plerixafor mobilizes cells having improved engraftability.

FIG. 25, right panel, shows the results of a secondary transplantation experiment where engrafted cells from the primary transplant experiment shown in FIG. 25, left panel, were harvested and then transplanted into recipient mice. The data show vastly improved engraftment rates for the MGTA-145 alone and MGTA-145+plerixafor cohorts as compared to the other cohorts and suggest that MGTA-145 alone or in combination with plerixafor mobilizes higher quality grafts that contain a higher proportion of long-term reconstituting HSCs relative to progenitor cells as compared to the other mobilizing regimens.

### Example 9: Expansion and/or Genetic Modification of Hematopoietic Stem and Progenitor Cells

Hematopoietic stem and progenitor cells (CD34+ cells) mobilized in Part D of the study were pre-stimulated for 1 day prior to electroporation with gRNA targeting beta-2 microglobulin (B2M) and Cas9. After electroporation (EP), cells were cultured for 1 or 7 days in the presence or absence of an aryl hydrocarbon receptor antagonist (“AHR;” 500 nM). Controls included were:

Mock Control: These cells were not electroporated and were used to assess growth capabilities of MGTA-145/p mobilized blood±AHR.

Mock EP Control: No gRNA or Cas9 was included in this group which was used to assess cell number and viability after EP.

Minimally Manipulated Control: 1-day culture to approximate culture conditions used by most gene therapy groups for CRISPR-Cas9 based editing strategies.

G-CSF mobilized blood was used as a comparator, because HSCs from G-CSF mobilized blood have been previously shown to be capable of gene-modification and expansion with AHR (Hoban et al. ASGCT 2019).

TABLE 17
Study Outline by Day.

| Study Day | Action |
|---|---|
| Day 0 | Thaw cells |
| | Pre-stimulate cells in cytokine-containing |
| | media (±AHR) for 1 day |
| Day 1 | Electroporate (EP) samples according to |
| | TABLE 18 using B2M gRNA + Cas9 |
| | Culture cells for 1 (minimally |
| | manipulated) or 7 days (±AHR) after EP |
| Day 2 or 8 | Phenotype cells with HSPC markers |
| | (CD34, CD90, CD45RA, 7-AAD) and |
| | B2M to assess knockout efficiency in |
| | total nucleated cells (TNC), CD34+ cells, |
| | and CD34+ CD90+ CD45RA− cells) |

TABLE 18
Treatment Arms

| | | | | Days in | | | n |
|---|---|---|---|---|---|---|---|
| Treatment | | gRNA/ | | Culture | | Number | repli- |
| Arm | EP | Cas9 | AHR | Post-EP | Donors | of Cells | cates |
| Mock DMSO | − | − | − | 7 | 1 | 50,000 | 2 |
| Mock AHR | − | − | + | 7 | | | 2 |
| Mock Pulse + | + | − | − | 7 | | | 2 |
| DMSO | | | | | | | |
| Mock Pulse + | + | − | + | 7 | | | 2 |
| AHR | | | | | | | |
| Minimally | + | + | − | 1 | | | 2 |
| Manipulated (1 | | | | | | | |
| day culture) | | | | | | | |
| Edit (B2M) + | + | + | − | 7 | | | 2 |
| DMSO | | | | | | | |
| Edit (B2M) + | + | + | + | 7 | | | 2 |
| AHR | | | | | | | |

Number of CD34+ and CD34+CD90+ cells and frequency of editing (beta-2-microglobulin negative) was assessed by flow cytometry.

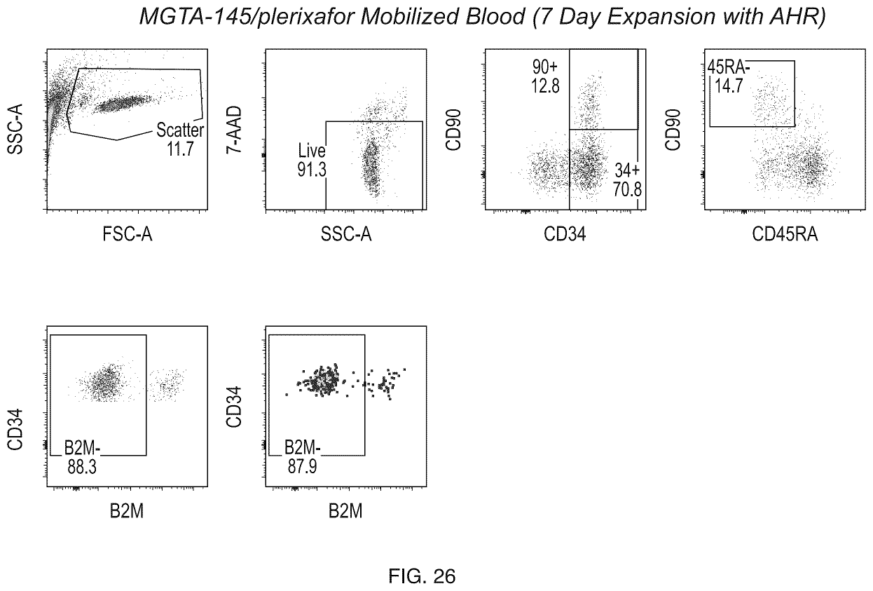

FIG. 26 provides a representative gating scheme for evaluation of MGTA-145/plerixafor-mobilized blood.

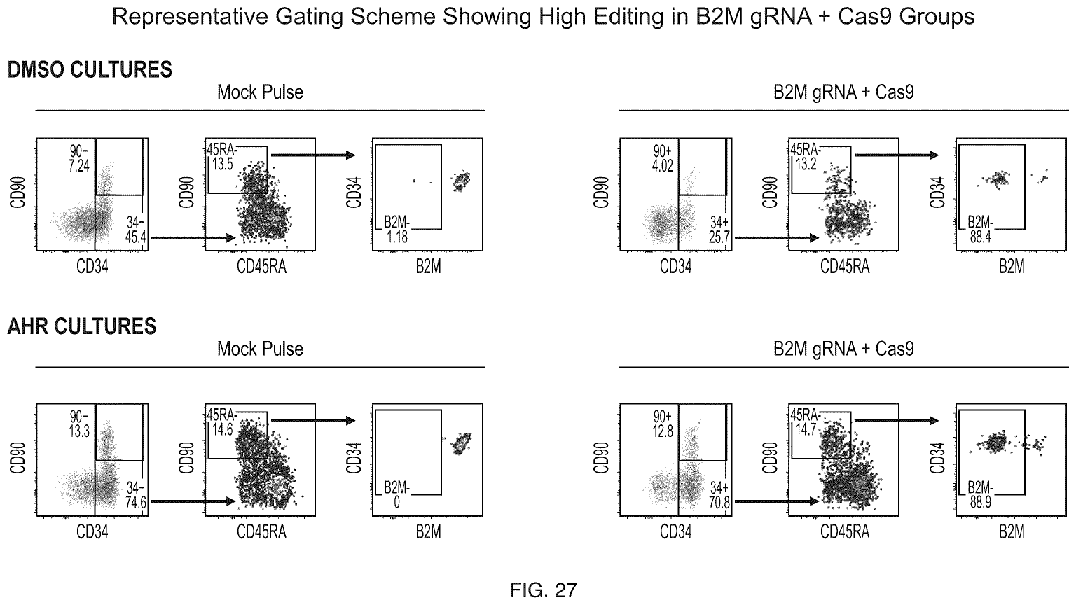

FIG. 27 provides a representative gating scheme showing high editing in B2M gRNA+Cas9 groups (both DMSO cultures and AHR cultures).

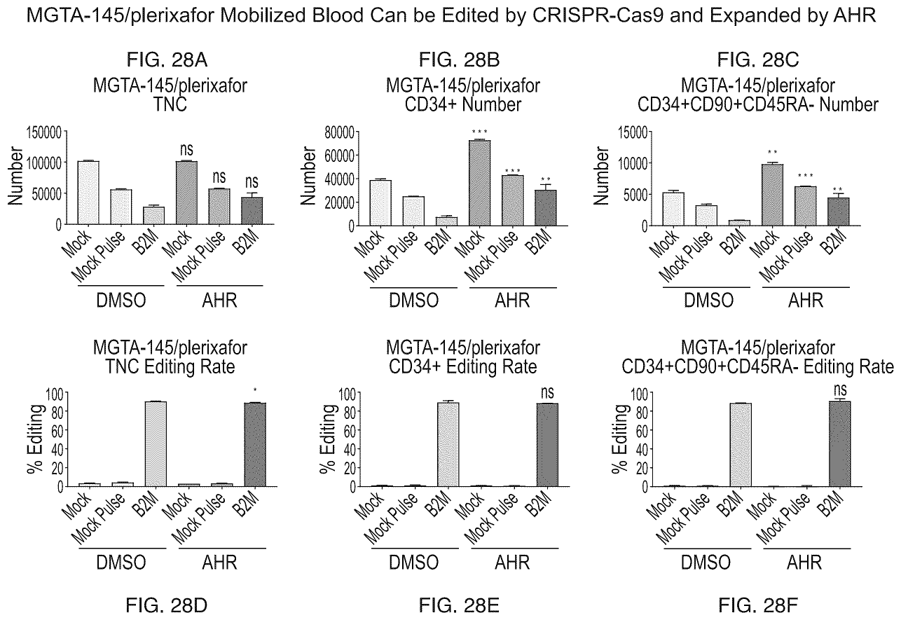

FIG. 28 provides bar graphs showing that MGTA-145/plerixafor-mobilized blood can be edited by CRISPR-Cas9 and expanded by AHR. Specifically, as shown in FIGS. 28B and C, addition of AHR increases the numbers of CD34+ cells (FIG. 28B) and CD34+CD90+CD45RA− cells (FIG. 28C) under all conditions (mock, mock pulse, and B2M), as compared to control TNC cells (FIG. 28A). Further, as shown in FIGS. 28D-F, TNC cells (FIG. 28D), CD34+ cells (FIG. 28E) and CD34+CD90+CD45RA− cells (FIG. 28F) were all edited by CRISPR-Cas9 under the conditions tested.

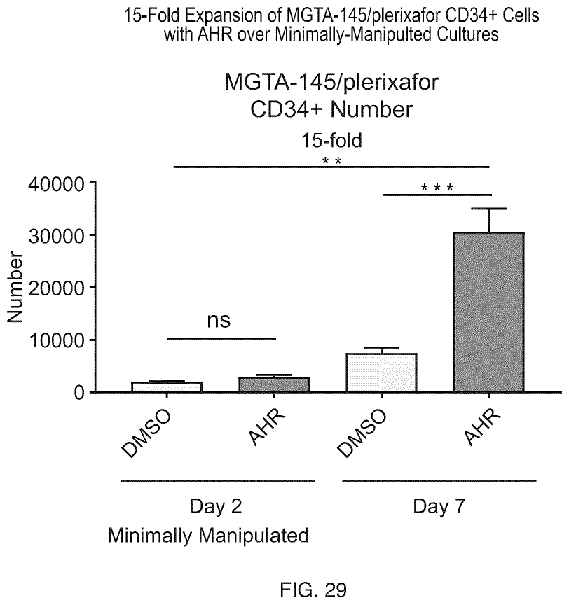

FIG. 29 provides a bar graph showing that a 7-day culturing protocol with AHR results in a 15-fold expansion of CD34+ cells over the typical 2-day culturing protocol typically used for CRISPR-Cas9 editing (i.e., 1 day pre-stimulation prior to electroporation with a gRNA and Cas9, followed by a 1 day post-EP culture.

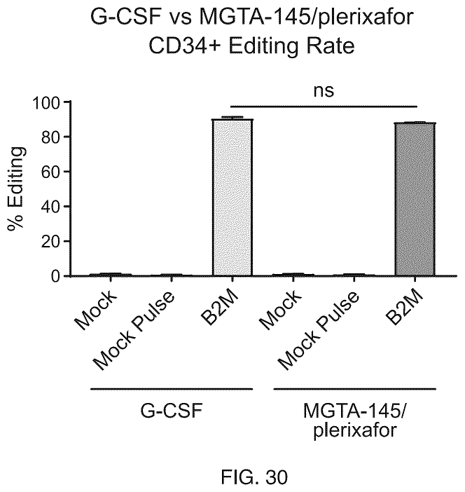

FIG. 30 provides a bar graph showing that there is no difference in editing rates between G-CSF-mobilized CD34+ cells and MGTA-145/plerixafor-mobilized CD34+ cells.

This example shows that high gene editing rates are achieved (˜90%) across all samples, indicating that MGTA-145/plerixafor mobilized blood can be edited to similar levels as G-CSF mobilized blood. Further, a 15-fold expansion of MGTA-145/plerixafor CD34+CD90+CD45RA− mobilized blood cells was observed using AHR over minimally-manipulated cultures, indicating that MGTA-145/plerixafor-mobilized blood can be expanded by AHR to similar levels as G-CSF-mobilized blood.

### Example 10: MGTA-145+Plerixafor CD34+ Cells from Humans can be Efficiently Gene Modified and En-Grafted in NSG Mice

This example demonstrates that MGTA-145+plerixafor CD34+ cells from humans can be efficiently gene modified and engrafted in NSG mice.

Mobilized blood cells (MGTA-145/p or G-CSF) were pre-stimulated for 1 day prior to electroporation with gRNA B2M. After electroporation, cells were cultured for 1 or 7 days±AHR, using the same control arms as assessed in vitro (see Example 9). An “uncultured” arm was transplanted to assess engraftment capabilities of cells that were not cultured.

TABLE 19
Study Outline by Day.

| Study Day | Action |
|---|---|
| Day 0 | Thaw cells |
| | Transplant uncultured (“fresh”) cells |
| | Pre-stimulate cells in cytokine-containing |
| | media (±AHR) for 1 day |
| Day 1 | Electroporate (EP) samples according to |
| | TABLE 20 using B2M gRNA + Cas9 |
| | Culture cells for 1 (minimally |
| | manipulated) or 7 days (±AHR) after EP |
| Day 2 or 8 | Phenotype cells with HSPC markers |
| | (CD34, CD90, CD45RA, 7-AAD) and |
| | B2M to assess knockout efficiency in |
| | total nucleated cells (TNC), CD34+ cells, |
| | and CD34+ CD90+ CD45RA− cells) |

TABLE 20
Treatment Arms

| | | | | Days in | | | n |
|---|---|---|---|---|---|---|---|
| Treatment | | gRNA/ | | Culture | | Number | repli- |
| Arm | EP | Cas9 | AHR | Post-EP | Donors | of Cells | cates |
| Mock DMSO | − | − | − | 7 | 1 | 50,000 | 2 |
| Mock AHR | − | − | + | 7 | | | 2 |
| Mock Pulse + | + | − | − | 7 | | | 2 |
| DMSO | | | | | | | |
| Mock Pulse + | + | − | + | 7 | | | 2 |
| AHR | | | | | | | |
| Minimally | + | + | − | 1 | | | 2 |
| Manipulated | | | | | | | |
| (1 day culture) | | | | | | | |
| Edit (B2M) + | + | + | − | 7 | | | 2 |
| DMSO | | | | | | | |
| Edit (B2M) + | + | + | + | 7 | | | 2 |
| AHR | | | | | | | |

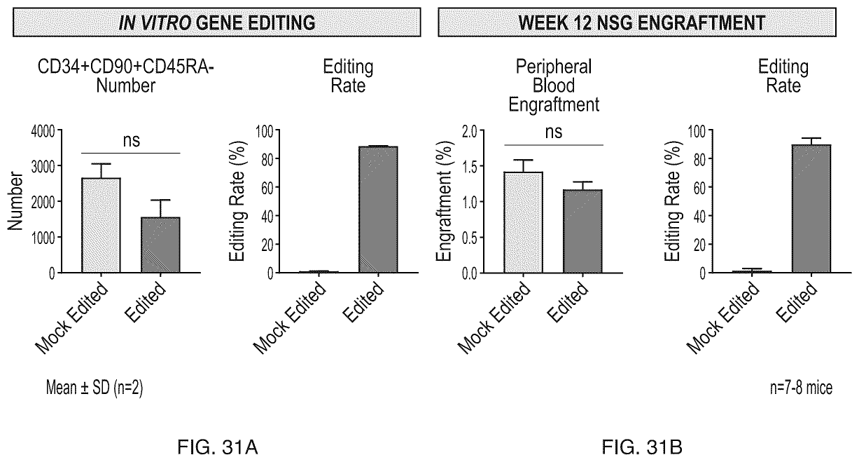

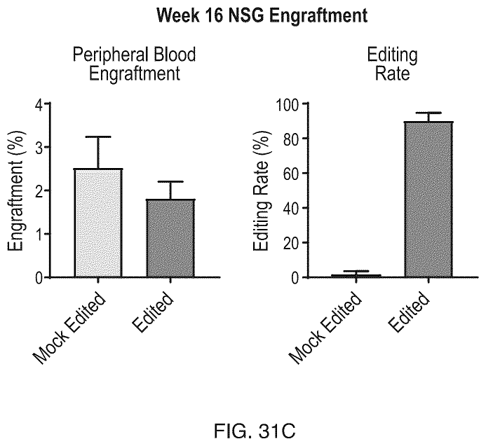

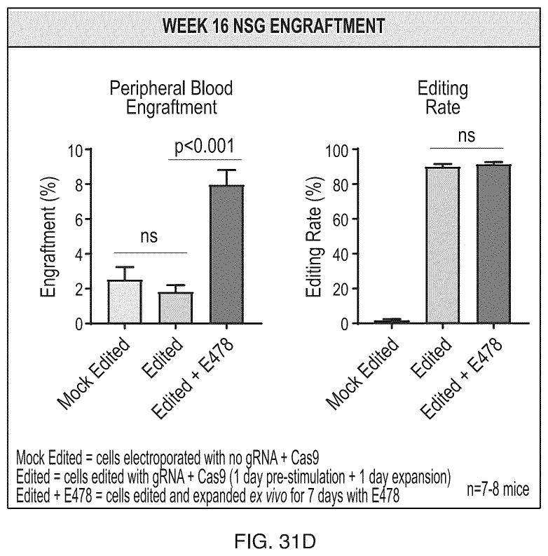

As shown in FIGS. 31A-C, B32M gene edited cells were efficiently gene edited and engraftment of edited cells occurred at a similar rate as compared to mock edited cells.

### Example 11: Evaluation of the Pharmacokinetics and Safety and Tolerability of MGTA-145 in Subjects with Normal Estimated Glomerular Filtration Rate (GFR) and Varying Degrees of Renal Impairment

This clinical trial will have three arms: donors with normal kidney function, donors with a mild decrease in GFR, and donors with a moderate decrease in GFR. Donors will be administered a single dose of MGTA-145 at 0.07 mg/kg intravenously.

Samples of peripheral blood will be taken at intervals following administration of MGTA-145 and Pharmacokinetics Biomarkers will be determined, including area under the curve (AUC), maximum plasma concentration (Cmax), clearance (CL), volume of distribution at steady state (Vdss), half-life, and renal clearance of MGTA-145. Safety and tolerability of MGTA-145 will be assessed by determining the occurrence of adverse events and administering clinical laboratory test, and taking vital signs and ECGs.

The following describes the inclusion criteria for donors used in this example.

### Donors:

Age from 18 to 79 years.
Body weight ≥50 kg and body mass index 19 to 40 kg/m².
Systolic blood pressure ≤170 mmHg and diastolic blood pressure ≤100 mmHg at Screening and Day 1.
No clinically significant abnormalities on physical examination at Screening.
Alanine aminotransferase and aspartate aminotransferase up to 1.5×the upper limit of normal (ULN) as long as total bilirubin and alkaline phosphatase are ≤ULN.
No clinically significant abnormalities on ECG and QTcF <480 msec at Screening.
Female donors are not pregnant, non-lactating, and must be of nonchildbearing potential.
Male donors who are sexually abstinent or surgically sterilized (vasectomy), or agree to use an acceptable method of contraception.
Donors using medications known to affect the elimination of serum creatinine (e.g., cimetidine, trimethoprim) within the past 30 days
Capable of providing informed consent and willing to comply with the requirements of the protocol.

Specific inclusion criteria for donors with normal renal function includes:
Estimated GFR (based on MDRD equation) ≥90 mL/min/1.73 m² (normal) as determined by an average of 2 values obtained at least 48 hours apart within the previous 3 months.
White blood cell (WBC) count, hemoglobin and platelet count within normal limits. Absolute neutrophil count of >1500/μL for African Americans and >2000/μL for other races.

Specific inclusion criteria for donors with renal impairment includes:
Estimated GFR <90 mL/min/1.73 m2 (based on MDRD equation) as determined by an average of 2 values obtained at least 48 hours apart and within the previous 3 months.
Stable renal function as determined by <20% difference in serum creatinine obtained on 2 occasions at least 48 hours apart and within the previous 3 months.
Platelet count ≥100,000/mm³, hemoglobin count ≥10 g/dL, WBC count within normal limits. Absolute neutrophil count of >1500/μL for African Americans and >2000/μL for other races.

Selected donors do not have any clinically significant laboratory value outside the normal range at screening. Selected donors do not have a history of alcoholism or drug abuse within the past 3 years. Selected donors do not have a history of kidney transplantation or requiring dialysis or anticipated to initiate dialysis during the study period. Selected donors have not donated more than 500 mL of blood or plasma within 12 weeks prior to dosing. Subject does not smoke more than 10 cigarettes per day and has not done so within 6 months prior to the screening visit. Selected donors have not had acute illness, infection (requiring medical treatment [e.g., antibiotics]), or surgery within 4 weeks of dosing. Selected donors are not seropositive for hepatitis B surface antigen (HBsAg), hepatitis C virus (HCV) antibody, or human immunodeficiency virus (HIV). Selected donors have not received another investigational drug or participated in an investigational drug or device study within 12 weeks prior to dosing. Selected donors do not have a history of anaphylaxis or clinically important reaction to any drug including plerixafor.

Selected donors having normal renal function will not have any clinically significant laboratory value outside the normal range at Screening. Selected donors with normal renal function do not have any clinically significant laboratory value outside the normal range at screening nor any hematologic, cardiovascular, pulmonary, central nervous system, metabolic, renal, hepatic, or gastrointestinal conditions that may put subject at risk or interfere with study results. Selected donors with normal renal function have not used any prescription drugs within 14 days prior to dosing or any dietary supplements or non-prescription drugs within 7 days prior to dosing unless deemed acceptable.

Selected donors having renal impairment will not have acute kidney injury, clinically significant laboratory abnormalities excluding those associated with renal impairment or the underlying cause of renal disease, an unstable medical condition or underlying medical condition that has changed within the past 90 days, presence of laboratory abnormalities or clinically significant medical condition that may place the subject at an unacceptable risk as a participant in this study or may interfere with the interpretation of the study results, or changes in prescription medications within 14 days prior to dosing or anticipated changes during the study period.

It is expected that administration according to the Example will rapidly give rise to populations of cells that are enriched in CD34⁺CD90⁺CD45RA⁻ cells, as described in more detail herein. Furthermore, it is expected that the administration of MGTA-145 will be safe and effective, even for donors with mild and moderate decreases in GFR.

### Example 12: CD14+ Myeloid Cells from MGTA-145+Plerixafor Grafts Contribute, at Least in Part, to Immunosuppression

The goal of this example was to determine whether monocytes contribute to immunosuppressive properties of MGTA-145+plerixafor graft in vivo. CD14+ cells were depleted from MGTA-145+plerixafor peripheral blood mononuclear cells (PBMCs) by bead-based separation approaches and transplanted into sublethally-irradiated (200 cGy) NSG mice. Greater than 90% depletion of CD14+ cells were achieved (data not shown).

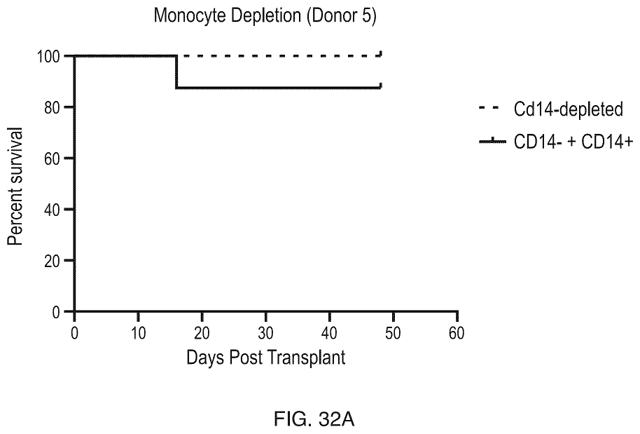

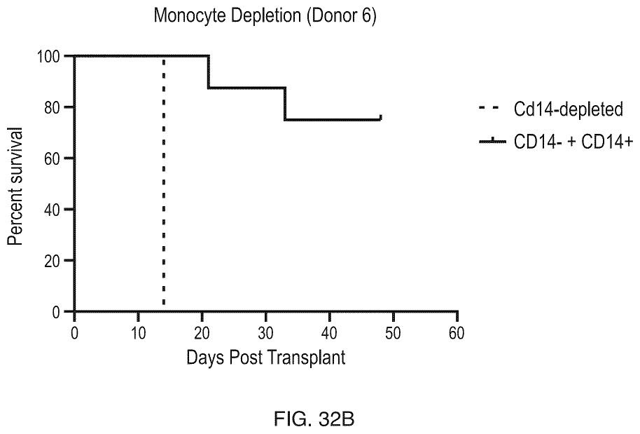

Survival was compared to non-depleted PBMCs (CD14−+CD14+). Input number was normalized to T cell content. FIGS. 32A and 32B depict survival of mice transplanted with CD14 depleted cells from two different donors. As shown in FIG. 32A, for Donor 5, no effect on survival time resulting from CD14 depletion was observed, likely due to a less xenoreactive graft. In contrast, as shown in FIG. 32B for Donor 6, all mice transplanted with CD14-depleted mice succumbed to GvHD. Greater than 70% of mice who underwent transplant of non-depleted CD-14 grafts exhibited survival beyond Day 40 post transplant.

Altogether, this suggests that CD14 myeloid cells contribute towards immunosuppression, and more specifically contributes towards GvHD prevention and immunosuppression.

### Example 13: Phase 2 Clinical Trial Methods for Autologous Stem Cell Transplant in Multiple Myeloma Patients

This example describes a dosing regimen involving MGTA-145 and plerixafor for mobilizing stem cells within patients with multiple myeloma such that the mobilized stem cells can be collected and used for an autologous stem cell transplant. In particular, this example describes a Phase 2 single center study for evaluating hematopoietic stem cell mobilization with MGTA-145+plerixafor and same day apheresis in patients with multiple myeloma.

Objectives were:
To assess the efficacy of MGTA-145 in combination with plerixafor in mobilizing adequate number of hematopoietic stem cells (>2×10⁶ CD34+ cells/kg) in patients with multiple myeloma (MM) in preparation for autologous stem cell transplantation (ASCT).
To assess the efficacy of MGTA-145 and plerixafor in mobilizing different Hematopoietic stem cells (HSCs) target goals in patients with MM in preparation for ASCT.
To assess the safety and tolerability of MGTA-145 and plerixafor for mobilizing HSCs in patients with MM.
To assess the engraftment rate and time to engraftment following ASCT after HSC mobilization with MGTA-145 and plerixafor in patients with MM undergoing upfront ASCT.
To assess rate of ongoing engraftment at day 30 and 100 after stem cell infusion in patients with MM who are mobilized with MGTA-145 and plerixafor undergoing upfront ASCT.
To assess transplant and disease-related outcomes after mobilization of HSCs with MGTA-145 and plerixafor in patients with MM undergoing upfront ASCT.

Eligible patients were defined as patients with myeloma 18-70 years of age, within 1 year of treatment start and creatinine clearance >30 ml/min. Patients received subcutaneous plerixafor (0.24 mg/kg or 0.16 mg/kg if renal dysfunction (creatine clearance ≤50 mL/min.), followed 2 hours later by intravenous MGTA-145 (0.03 mg/kg) over 3-10 minutes and apheresis within 30 minutes. Mobilization was repeated for a second day if day 1 yield was <6×10⁶ CD34+ cells/kg.

Primary endpoint is collection of 2×10⁶ CD34+ cells/kg in 1 or 2 days of mobilization/apheresis. Secondary endpoints include:
Collection of 4×10⁶ and 6×10⁶ CD34+ cells/kg and engraftment after transplant.
Evaluate infusion related toxicities with MGTA-145 over 7 days after mobilization. Adverse events were collected based on NCI CTCAE version 5 with mobilization of HSCs with MGTA-145 and plerixafor.
Evaluate neutrophil engraftment over 48 hours. Only patients proceeding with upfront transplant were assessed. Time to primary engraftment was defined as time in days from the day of stem cell infusion to the first day of engraftment. Neutrophil engraftment was defined as the first day of Absolute neutrophil count (ANC) ≥0.5×10⁹/L for 3 days following stem cell infusion. This was assessed by absolute neutrophil counts obtained as part of complete blood count (CBC) with differential. It is standard practice to follow patients regularly in the inpatient unit/outpatient transplant unit until they achieve engraftment.
Evaluate platelet engraftment over 48 hours. Only patients proceeding with upfront transplant were assessed. Time to primary engraftment was defined as time in days from the day of stem cell infusion to the first day of engraftment. Platelet engraftment was defined as the first day of platelet count more than or equal to 20×10⁹/L without transfusion in the last 7 days and with platelet count ≥20×10⁹/L on 2 separate, subsequent days. This was assessed by platelets counts obtained as part of CBC with differential. It is standard practice to follow patients regularly in the transplant inpatient unit/outpatient transplant until they achieve engraftment.
Transplant related mortality was assessed over 100 days after transplant. Only patients proceeding with upfront transplant were assessed. Transplant related mortality was described as death from any cause within the first 100 days of HSC infusion.
Non-relapse related mortality was assessed over 100 days after transplant. Only patients proceeding with upfront transplant were assessed. Transplant related non-relapse mortality was described as death from any cause except disease relapse/progression within the first 100 days of HSC infusion.
Progression Free Survival (PFS) was assessed over 100 days after transplant. Only patients proceeding with upfront transplant will be assessed. Duration from start of the ASCT to disease progression or death (regardless of cause of death), whichever comes first. Progression was assessed using day 100 post-transplant response and PFS rates were reported at day 100 in patients undergoing upfront transplant.
Overall Survival (OS) was assessed over 100 days after transplant. Only patients proceeding with upfront transplant were assessed. Overall survival was defined as duration from start of the ASCT to death (regardless of cause of death). This was assessed from start of transplant and OS rates were reported at day 100 following transplant in patients undergoing upfront transplant.
Response was assessed over 100 days after transplant. Response before mobilization was assessed in all patients. Response/progression following transplant was assessed in patients undergoing upfront transplant. Myeloma response before stem cell mobilization and at day 100 will be assessed per the International Myeloma Working Group Uniform Response Criteria.

Inclusion Criteria were as follows:
Diagnosis of multiple myeloma per the International Myeloma Working Group (IMWG) criteria
Eligible for ASCT per institutional guidelines
Within one year of start of myeloma therapy
Cardiac and pulmonary status sufficient to undergo apheresis and transplantation per institutional transplant guidelines.
Calculated creatinine clearance >30 mL/min according to the Modification of Diet in Renal Disease (MDRD) formula.
Absolute neutrophil count >1500×10⁶/L and platelets >100,000×10⁶/L
Ability to understand and the willingness to sign a written informed consent document.
Agreement to use an approved form of contraception for male patients or female patients of childbearing potential.

Exclusion Criteria were as follows:
History of prior stem cell transplant for multiple myeloma or other indications
Planned tandem stem cell transplant
Prior history of failure to collect HSCs.
Liver function tests: Total bilirubin >1.5× upper limit of normal (ULN) in the absence of a documented history of Gilbert's syndrome and/or aspartate aminotransferase (AST)/alanine aminotransferase (ALT) >3×ULN.
Known allergy to MGTA-145 or plerixafor.
Lifetime exposure to lenalidomide or another immunomodulatory drug greater than 6 cumulative months of treatment i.e. more than six 28-day cycles or more than eight 21-day cycles.
Pregnant or lactating women.

A total of 15 eligible patients were identified for stem cell mobilization. Plerixafor 0.24 mg/kg was administered in 14 patients and 0.16 mg/kg plerixafor was administered in 1 patient (renal dosing). Of the patients, median age was 62 years (range: 46-68) and 60% were female. Twenty-nine percent (29%) of the patients had International Staging System (ISS) 1, 36% had ISS 2 and 36% had ISS 3 (N=14, N/A in 1). Seven (7) (47%; N=13, N/A in 2) patients had high-risk fluorescence in situ hybridization test (FISH). Induction therapy was bortezomib+lenalidomide+dexamethasone (VRD) in 11 (73%) and daratumumab+VRD in 4 (27%) patients (3 patients started with cyclophosphamide, bortezomib and dexamethasone and switched to daratumumab+VRD). The patients' median induction duration was 4 months (3-6), median lenalidomide exposure was 6 cycles (1 to 7), with at least a very good partial response (VGPR) in 12 (80%). Twelve (12) patients (80%) had one line of therapy prior to mobilization and 3 patients (20%) had two lines of therapy prior to mobilization. Five (5) patients had at least one risk factor for poor mobilization (e.g., prior other cancer treated with chemotherapy and/or radiation, autoimmune disorder needing systemic therapy, daratumumab, alkylator exposure for more than 2 months). Six (6) of the patients were included in a safety cohort that underwent subsequent transplantation.

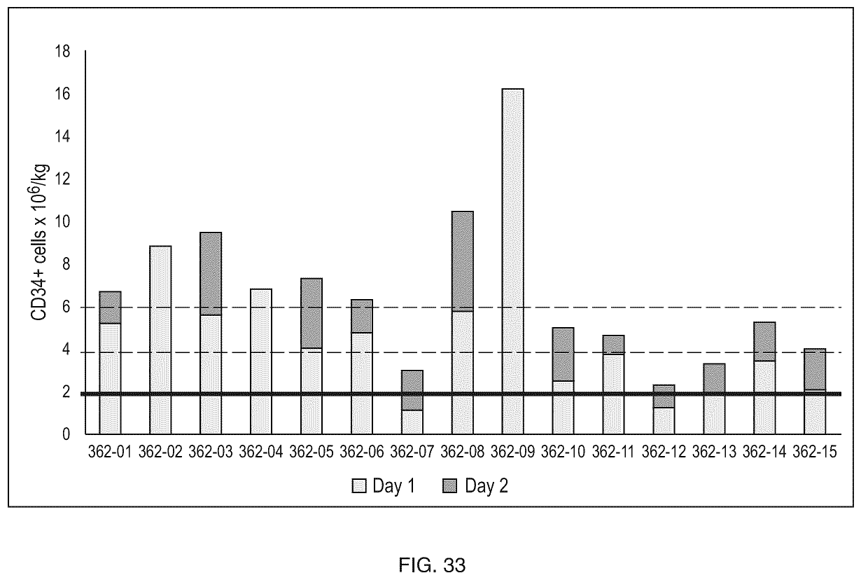

On Day 1, all 15 patients underwent MGTA-145+plerixafor mobilization, followed by apheresis, as disclosed above in Example 13. CD34+ cell yield in the apheresis products were quantified. As shown in FIG. 33, the apheresis product of 3 of the 15 multiple myeloma patients yielded greater than 6×10⁶ cells/kg. The other 12 patients underwent a day 2 MGTA-145+plerixafor mobilization followed by apheresis. FIG. 33 also shows the total CD34+ cell yield quantified across the one or two apheresis products obtained for each patient. The median total stem cell yield (CD34+ cells/kg×10⁶) was 6.3 (range=2.3-16.2). For the first 10 patients, the median day 1 yield was 5.4 (range=1.1-16.2), and yield per apheresis session was 4 (range=1.1-16.2). 100% of patients (15/15) met the primary endpoint of collecting sufficient HSCs in ≤2 days of mobilization+apheresis to proceed to transplant. Notably 80% of patients met the primary endpoint in 1 day (2×10⁶ CD34+ cells/kg). Secondary endpoints of 4 and 6×10⁶ CD34+ cells/kg in ≤2 days were met in 80% & 53% patients, respectively.

Alternate mobilization was needed for two patients: 1 for collecting backup stem cells and 1 for complications with product during cell processing and cryopreservation after collection.

MGTA-145 was well tolerated. At least 1 adverse event (AE) was seen in 90% of patients, 20% had grade 2 AEs (anemia, hypokalemia) and 20% had grade 3 AEs (worsening of baseline grade 3 anemia; hypocalcemia). All adverse events were resolved.

Treatment emergent adverse events, TEAEs (at least possibly related to MGTA-145) were seen in 60% of patients. Pain was experienced by 11 (50%) of patients, with acute pain in 9 of the patients (back-5, hip-1, sternum-1, generalized-1). One patient had mild shoulder pain during apheresis. The median severity of pain on a scale of 1-10 was an 8 (3-10). The acute and transient bone pain was categorized as grade 1, were all experienced on day 1, with an onset of about 5 minutes (median, range 3-10) after the start of MGTA-145 infusion, and with a median duration of 7 minutes (median, range 3-28). Two (2, 8%) patients experienced nausea, 1 (4%) patient experienced hyperhidrosis, and 2 (8%) patients experienced thrombocytopenia. One patient (4%) experienced vomiting, which was categorized as a grade 2 adverse event.

Twelve (12) out of 12 patients transplanted with MGTA-145+plerixafor-collected HSCs have successfully engrafted. Ten (10) of these patients completed the transplant with melphalan 200 mg/m². A median of 4 (2.4-8.1)×10⁶ CD34+ cells/kg were infused. All patients exhibited timely engrafts (Johnsrud et al. (2021) Transplant Cell Ther. S2666-6367(21)00863-0; DiPersio et al. (2009) Blood 113(23):5720-6). Specifically, median time to neutrophil engraftment was 12.5 days (range=11-15) median time to platelet engraftment (≥20,000×10⁶/L without transfusion in 7 days) was 18 days (range=15-33). One patient needed an RBC transfusion during the transplant.

Apheresis graft analysis is available in these 14 patients. Grafts with MGTA-145+plerixafor showed high enrichment for CD90+CD45RA− among CD34+ cells. CD90+CD45RA− cells represent a CD34 subset of long term engrafting HSCs (median: 40% of CD34+ cells, 18-66%). Here, the percentage of CD90+CD45RA− cells was higher than the percentage of CD90+CD45RA− that are observed when using a mobilization regiment that includes G-CSF (6%, Goncalves ASTCT 2021). 74% of grafts (10) were minimal residual disease (MRD) negative with next generation flow cytometry (sensitivity >1×10⁵). 2.5×10⁶ events were analyzed (median) with 0.0002% (0.0001-0.0004) median clonal plasma cells in MRD positive patients.

Six patients completed the day 100 follow up and all have exhibited durable engraftment.

Phase 2 Clinical Trial Expansion Cohort

The study as described above in Example 13 was an open-label single arm trial of 15 patients. Given that the single-arm trial met the requirement that 13 or more patients meet the primary endpoint, an expansion cohort of 10 patients was conducted. The trial has 85% power at a 5% one-sided type I error rate. The analysis herein is based on aggregated results from a total cohort of 25 patients.

As described above, a single center phase 2 trial of MGTA-145+plerixafor for HSC mobilization was conducted in patients with multiple myeloma (MM), NCT04552743. Primary endpoint was collection of ≥2×10⁶ CD34+ cells/kg in up to 2 days of mobilization/apheresis. Secondary endpoints were collection of 4 and 6×10⁶ CD34+ cells/kg, safety and engraftment. Patients with MM, 18-70 years of age, within 1 year of starting treatment & CrCl>30 ml/min were eligible.

Patients received plerixafor 0.24 mg/kg (0.16 mg/kg if renal dysfunction) SQ, followed 2 hours later by MGTA-145 (0.03 mg/kg) IV over 3-10 minutes and apheresis within 30 minutes. Mobilization and collection were repeated for a second consecutive day if day 1 yield was <6×10⁶ CD34+ cells/kg.

Results: Median age was 62 years, 52% were female, 24% had ISS stage 3, 57% had high-risk FISH (primarily gain1q). Induction therapy was VRD in 68% (17), daratumumab VRD in 24% (6), CyBorD in 8% (2) patients. Median duration of induction was 4 months (3-6) and median lenalidomide exposure was 5 cycles (1-8), with ≥VGPR in 88% of patients. (TABLE 21)

TABLE 21
Baseline Characteristics and Myeloma Treatment

| | N(%) or median (range) | |
|---|---|---|
| Age | 62 years | (35-68) |
| Sex, female | 13 | (52%) |
| Race (White, AA, Asian, Other/not known) | 18, 3, 2, 2 | |
| ISS stage 1, 2, 3, Not known | 6, 10, 6, 3 | |
| High-risk cytogenetics* (NA in 4) | 12 of 21 | (57%) |
| Renal Insufficiency at diagnosis | 7 | (28%) |
| Myeloma Treatment | | |
| Induction Chemotherapy:** | | |
| VRD (bortezomib, lenalidomide, dex) | 17 | (68%) |
| Daratumumab VRD | 6 | (24%) |
| CyBorD (cyclophosphamide, bortezomib, dex) | 2 | (8%) |
| Duration of induction, median (range) | 4 months | (3-6) |
| Lenalidomide exposure, N = 23 | 5 cycles | (1-8) |
| More than 2 months of cyclophosphamide** | 4 | (16%) |
| Lines of therapy before mobilization, 1 vs. 2 | 22, 3 | (88% , 12%) |
| Radiation therapy for myeloma | 5 | (20%) |

*High-risk cytogenetics: Deletion 17p/monosomy 17; t(4; 14), t(14; 16), t(14; 20) and gain 1q
**3 patients started with Cy BorD and switched to DaraVRD (1) or VRD (2)

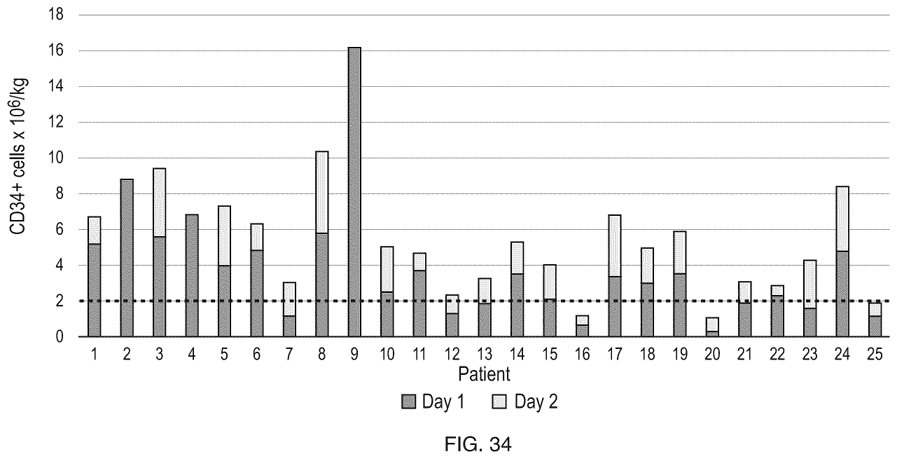

Plerixafor 0.24 mg/kg was given in 24 (96%) patients. Median pre-apheresis CD34 count was day 1, 24/μL (3-99) & day 2, 15/μL (5-46). Median total HSC cell yield (CD34+ cells/kg×10⁶) was 5.0 (1.1-16.2), day 1 yield was 3.4 (0.3-16.2) and day 2 yield was 1.9 (0.5-4.6) (FIG. 34). 88% (n=22) of patients met the primary endpoint of collecting sufficient HSCs in ≤2 days of mobilization+apheresis to proceed to transplant, 68% (17) in 1 day (2×10⁶ CD34+ cells/kg). Three (3) patients who did not meet primary endpoint successfully collected HSCs with standard GCSF+plerixafor dosing & 2-3 apheresis sessions. Secondary endpoints of 4 and 6×10⁶ CD34+ cells/kg in ≤2 days were met in 68% (17) and 40% (10) patients.

MGTA-145+plerixafor was well tolerated. At least 1 treatment emergent AE (TEAE) with MGTA-145 was seen in 60% of patients (grade 1, n=14, grade 2, n=1). Pain (all grade 1) was most common, seen in 44% (11), with 38% (9) patients experiencing acute onset transient bone pain with MGTA-145 (duration: 7 minutes, range: 3-28). Using the validated Brief Pain Inventory, 56% (14) patients self-reported pain with mobilization vs 40% (10) at baseline. 7/15 patients without baseline pain reported pain with mobilization. An increase for worst pain was seen on mobilization day 1, that returned to the baseline, but no difference for aggregate score of pain severity/interference (linear mixed effects model).

At last follow-up, 18 patients have completed transplant with MGTA-145 mobilized graft, with melphalan 200 mg/m² in 15 (83%) patients. Median of 3.5 (2.2-8.1)×10⁶ CD34+ cells/kg were infused. All patients have engrafted. Median time to neutrophil engraftment of 12 days (range: 11-15) & platelet engraftment (platelets ≥20,000×10⁶/L, no transfusion in 7 days) of 17.5 days (15-33) are comparable to historical data (DiPersio et al. 2009). RBC transfusion was needed in 3 (17%) patients. 14 patients have day 100 follow-up, all with durable engraftment.

MGTA-145+plerixafor mobilized grafts had a favorable graft composition as evaluated using flow cytometry for HSC immunophenotyping. High enrichment was observed for CD90+CD45RA− among CD34+ cells, a CD34 subset of long term engrafting HSCs (median: 36% of CD34+ cells, range 10-66%, N=25). 74% (17 of 23) grafts were minimal residual disease negative with next generation flow cytometry.

Altogether, this is the first study to evaluate the novel regimen of MGTA-145+ plerixafor for same day stem cell mobilization and collection in myeloma/hematologic malignancies. The study cohort was representative of transplant eligible patients with MM, with 88% patients meeting the primary endpoint. The regimen was well tolerated. Patients achieved timely & durable engraftment. This example suggests that administration of MGTA-145 and plerixafor allows for the collection of a sufficient number of CD34+ cells for a transplant in a multiple myeloma patient, for example, after a single dose and/or a single-day mobilization regimen.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1.-129. (canceled)

130. A method of mobilizing a population of hematopoietic stem or progenitor cells from the bone marrow of a mammalian donor into peripheral blood, the method comprising administering to the donor a CXCR2 agonist selected from the group consisting of Gro-β, Gro-β T, and variants thereof at a dose of from about 0.001 mg/kg to about 0.1 mg/kg, wherein the CXCR2 agonist is administered over a period of time of less than about 20 minutes.

131. The method of claim 130, wherein the dose is from greater than about 0.015 mg/kg to less than about 0.05 mg/kg.

132. The method of claim 130, wherein the CXCR2 agonist comprises Gro-β T.

133. The method of claim 130, wherein the CXCR2 agonist is administered at a dose of about 0.03 mg/kg.

134. The method of claim 130, wherein the CXCR2 agonist is administered intravenously.

135. The method of claim 130, the method further comprising administering to the donor a CXCR4 antagonist.

136. The method of claim 135, wherein the CXCR4 antagonist is plerixafor.

137. The method of claim 136, wherein the plerixafor is administered to the donor at a dose of about 160 μg/kg.

138. The method of claim 137, wherein the donor has renal dysfunction.

139. The method of claim 138, wherein the donor has mild or moderate reduction in glomerular filtration rate (GFR).

140. The method of claim 136, wherein the plerixafor is administered to the donor at a dose of about 240 μg/kg.

141. The method of claim 135, wherein the CXCR2 agonist is administered simultaneously with the CXCR4 antagonist.

142. The method of claim 135, wherein the CXCR2 agonist is administered after the CXCR4 antagonist.

143. The method of claim 142, wherein the CXCR2 agonist is administered within about 4 hours of administration of the CXCR4 antagonist.

144. The method of claim 143, wherein the CXCR2 agonist is administered about 2 hours after the CXCR4 antagonist.

145. The method of claim 135, wherein the CXCR2 agonist and the CXCR4 antagonist are each administered on two consecutive days.

146. The method of claim 145, wherein the CXCR2 agonist and the CXCR4 antagonist are each administered once per day on two consecutive days.

147. The method of claim 130, wherein the CXCR2 agonist is administered over a period of time of less than about 10 minutes.

148. The method of claim 130, wherein the CXCR2 agonist is administered over a period of time from about 3 to about 10 minutes.


SEQUENCE LISTING (4 sequences)

SEQ ID NO: 1 - protein, 73 aa, Homo sapiens
APLATELRCQCLQTLQGIHLKNIQSVKVKSPGPHCAQTEVIATLKNGQKACLNPASPMVKKIIEKMLKNGKSN

SEQ ID NO: 2 - protein, 69 aa, synthetic construct
  REGION 1..69: note = Synthetic Construct
TELRCQCLQTLQGIHLKNIQSVKVKSPGPHCAQTEVIATLKNGQKACLNPASPMVKKIIEKMLKNGKSN

SEQ ID NO: 3 - protein, 73 aa, Homo sapiens
APLATELRCQCLQTLQGIHLKNIQSVKVKSPGPHCAQTEVIATLKNGQKACLNPASPMVKKIIEKMLKDGKSN

SEQ ID NO: 4 - protein, 69 aa, synthetic construct
  REGION 1..69: note = Synthetic Construct
TELRCQCLQTLQGIHLKNIQSVKVKSPGPHCAQTEVIATLKNGQKACLNPASPMVKKIIEKMLKDGKSN